(19) United States
(12) Patent Application Publication
(10) Pub. No.: US 2011/0217317 A1
(43) Pub. Date: Sep. 8, 2011

(54) TREATMENT OF THROMBOCYTOPENIA

(75) Inventors: Christian Meyer, Humlebaek (DK); Anne Marie Valentin Jensen, Copenhagen OE (DK); Ewa Lindenstroem, Vaerloese (DK); An Van Es-Johansson, Stockholm (SE)

(73) Assignee: Symphogen A/S, Lyngby (DK)

(21) Appl. No.: 13/061,015

(22) PCT Filed: Aug. 26, 2009
(86) PCT No.: PCT/DK2009/050214

§ 371 Date: May 23, 2011

Related U.S. Application Data
(60) Provisional application No. 61/136,314, filed on Aug. 27, 2008.

(30) Foreign Application Priority Data
Aug. 26, 2008 (DK) PA 2008 01 166

(51) Int. Cl. A61K 39/395; C07K 16/34; C12P 21/00; A61P 7/00

(52) U.S. Cl. 424/172.1; 530/389.8; 435/69.6

(57) ABSTRACT

The present invention relates to treatment of thrombocytopenia with a pharmaceutical composition comprising re-combinant polyclonal anti-RhesusD antibody product as the active ingredient.

All patent and non-patent references cited in the present application are hereby incorporated by reference in their entirety.

### FIELD OF INVENTION

The present invention relates to pharmacological and diagnostic compositions comprising anti-RhD recombinant polyclonal antibody product and their use in treatment of thrombocytopenia. The treatment of thrombocytopenia can be symptomatic, ameliorating, prophylactic and/or curative. The anti-RhD recombinant polyclonal antibody product and the production thereof is disclosed in PCT/DK2005/000501.

### BACKGROUND OF INVENTION

The Rhesus blood group antigens are located on transmembrane erythrocyte proteins encompassing the so-called C, c, E, e and D antigens. Approximately 16% of the Caucasian population is Rhesus D negative (RhD(−)) due to an inherited polymorphism. In addition, multiple genetic and serological variants of RhD exist (divided into category II-VII) of which RhD^{VI} is the most clinically relevant. Since category VI positive red blood cells (RBC) carry fewer of the various epitopes of the D protein than RBC of other categories, RhD^{VI}(+) individuals may form alloantibodies against RBC from other RhD positive (RhD(+)) individuals (Issitt, P. D. and Anstee, D. J., 1998. The Rh Blood Group System, Montgomery Scientific Publications, Durham, N.C., pp. 315-423).

ITP is a hematological disorder, where autoantibodies result in an accelerated platelet clearance in the spleen and liver. The incidence of ITP is estimated to be between 50 and 100 new cases per million. Anti-D immunoglobulin has proven useful in the treatment of idiopathic thrombocytopenic purpura (ITP) (George, J. N., 2002. Blood Rev. 16, 37-38). Corticosteroids and intravenous immunoglobulin (IVIg) usually constitute first-line therapy but blood donor-derived anti-Rhesus D immunoglobulin has proven both safe and effective and is being increasingly used as first-line treatment in ITP. In severe cases the spleen is removed. This is however, not possible in infants due to severe side effect, thus alternative treatments like anti-D immunoglobulin are needed.

ITP is defined by platelet counts <150×10⁹/L [150,000/mm³] and is characterized by increased bruising tendency. However, ITP often presents as spontaneous bleeding in individuals with platelet counts of less than 20×10⁹/L [20,000/mm³]. Patients with platelet counts <10×10⁹/L [10,000/mm³] may present with severe cutaneous bleedings, gingival bleeding, epistaxis, hematuria or menorrhagia. Spontaneous intracranial bleeding and other internal bleeding can be seen in severe thrombocytopenia with platelet counts below 5×10⁹/L [5,000/mm³](Stasi 2004). ITP in individuals with platelet counts above 30×10⁹/L [30,000/mm³] is most often diagnosed incidentally after a routine complete blood cell count. ITP is also characterized by an increased proportion of immature peripheral platelets, and to some extent by an increased proportion of megakaryocytes in the bone marrow. The clinical features of ITP in adults differ from those seen in childhood, in which spontaneous remissions occur in approximately 80% of patients. While ITP in children is usually an acute disease occurring two to three weeks after a viral infection, ITP in adults typically has an insidious onset and a chronic course. In addition, secondary forms of the disease also exist.

### Mechanism of Action in ITP

ITP is mediated by autoantibodies that are directed against platelet surface antigens. The autoantibody-opsonized platelets are rapidly eliminated by phagocytes of the RES causing thrombocytopenia. The major, although not the only, site for platelet elimination through the RES involves the spleen and the spleen is also considered to be the major site for further progression and amplification of the anti-platelet autoimmune antibody response (Cines 2002).

Currently the most accepted mechanism of action for anti-D immunoglobulin in ITP appears to be competitive blockade of Fcγ receptors (FcγR) in the RES, most likely FcγRII and III on phagocytic macrophages that have a high avidity for IgG-opsonized particles and IgG immune complexes. This mechanism of action for anti-D was initially proposed as a result of the effectiveness of IVIg in the treatment of ITP. The hypothesis has been further supported by the efficacy of anti-D immunoglobulin in RhD⁺ ITP patients and lack of efficacy in patients who are RhD⁻. The fact that splenectomised patients do not respond to anti-D treatment is evidence that anti-D exerts its effect in the RES/spleen environment (Lazarus 2003; Salama 1983; Salama 1984).

Blockade of FcγR-mediated platelet phagocytosis may, however, not be the sole mechanism to account for all the therapeutic benefit provided by anti-D treatment. One case of successful treatment of an RhD⁻ patient with anti-D immunoglobulin has been described. Considering the fact that the anti-D specific antibodies only constitute about 1% of the total amount of immunoglobulin in the currently available plasma derived anti-D products, alternative immunomodulatory mechanisms as proposed for the efficacy of IVIg cannot be fully excluded (Lazarus 2003).

### Current Therapies for ITP

Corticosteroids and IVIg are primarily used for treatment of ITP (Stasi 2004) as recommended in the ITP treatment guidelines formulated by the American Society of Hematology in 1996 (George 1996). Anti-D has proven both safe and effective (Blanchette 1994; Bussel 1991; Cooper 2002; Newman 2001; Sandler 2001; Scaradavou 1997) and is being increasingly used as a first-line treatment option in ITP (Cines 2005). Generally, patients with platelet counts above 30×10⁹/L [30,000/mm³] do not require treatment unless they are undergoing procedures which are likely to induce blood loss (George 1996). Approximately two-thirds of the patients will respond to prednisolone (1 mg/kg/day for 2-4 weeks). IVIg has also been demonstrated to effectively elevate platelet counts in 75% of the cases, half of which will achieve normal platelet counts. However, responses are transient and there is little evidence of a lasting effect. IVIg is given at a high dose of 400 mg/kg for five days or 1 g/kg for two days (1; 4). Cases failing to respond to first-line therapy or requiring unacceptably high doses of corticosteroids are defined as refractory ITP. High-dose corticosteroids have been used as second-line therapy in patients with refractory ITP, as wells as high-dose IVIg (e.g. 1 g/kg/day), often in combination with corticosteroids (Stasi 2004).

Splenectomy reduces antibody-mediated clearance of platelets, although extrasplenic RES tissue (e.g. liver) may propagate the disease (Cines 2005). Two-thirds of patients with ITP who undergo splenectomy will achieve a normal platelet count, which is often sustained with no additional therapy.

Treatment with anti-D may eliminate or significantly postpone the need for splenectomy, which is undertaken after other treatment strategies have been tested and found to fail. Intravenous anti-D has been shown to elevate the platelet counts in 60-90% of adults depending on success criteria (Bussel 1991; Newman 2001; Scaradavou 1997; Bussel 2001).

### Rationale for a Recombinant Polyclonal Anti-D Preparation

The anti-D immunoglobulin products currently available on the market comprise immunoglobulins obtained from human anti-D hyperimmune blood donors with only a small fraction being anti-D specific. These preparations contain antibodies directed against all major RhD categories in the human population.

Furthermore, RhD immunoglobulin from human plasma contains, apart from anti-D IgG, low titers of other blood group antibodies, and it has recently been suggested that some of the rare but serious acute adverse reactions of hemoglobinemia or hemoglobinuria following receipt of anti-D immunoglobulins for ITP treatment, could be caused by passively acquired blood group antibodies other than those reacting with RhD (Gaines 2005 and Schwartz 2006).

### Rhophylac® and WinRho®

Rhophylac® and WinRho® are both plasma-derived anti-D products, which consist of mixed, irrelevant human blood-derived IgG immunoglobulin and a small fraction of Rhesus D-specific antibodies. The potency of these products is determined against a WHO International Reference Preparation (International standard for minimum potency of the anti-D blood grouping reagents 2005)

WinRho® has proven to be useful in treatment of idiopathic thrombocytopenic purpura (ITP). However, WinRho® should not be administered to Rhₒ(D) negative patients or splenectomized patients. In the WinRho® labelling the following is stated: “If the patient has a lower than normal hemoglobin level (less than 10 g/dL), a reduced dose of 125 to 200 IU/kg (25 to 40 μg/kg) body weight should be given to minimize the risk of increasing the severity of anemia in the patient. A drug warning (FDA drug warning Dec. 5, 2005) for WinRho® has been issued regarding intravascular haemolysis and disseminated intravascular coagulation (DIC).

Idiopathic thrombocytopenic purpura (ITP) can also be treated with Rhophylac®. However, a drawback of Rhophylac® is that patients with preexisting anemia have to weigh the benefits of Rhophylac® against the potential risk of increasing the severity of the anemia.

In addition there are reports in literature that serum-derived anti-D leads to haemolysis in ITP patients and that this is the main adverse event (Scaradavou et al, 1997, Intravenous Anti-D treatment of immune thrombocytopenic purpura: Experience in 272 patients, Blood 89:2689-2700). Finally, a study in healthy volunteers concludes that there is a statistically significant linear trend between increasing doses of anti-D and haemolysis (Zunich et al, A dose ranging evaluation of the effect of a single administration of RH(D) globulin intravenous in healthy volunteers, Abstract #2641, Blood 1994; 84 (Suppl):664a).

Compared to plasma derived anti-D, individual anti-RhD monoclonal antibodies have shown to induce a more heterogeneous and slower clearance rate of erythrocytes from the circulation following experimental administration of RhD⁺ erythrocytes to RhD⁻ subjects. Thus, administration of monoclonal anti-D antibodies results in prolonged erythrocyte half life compared with polyclonal anti-D products (Kumpel 1995; Kumpel 2003; Miescher 2004) Reports indicate that monoclonal anti-D antibodies show no efficacy in ITP patients (summarised in Scaradavou et al, 1997) and at least one report (Godeau et al, 1997, Treatment of chronic autoimmune thrombocytopenic purpura with monoclonal anti-D, Transfusion, 36:328-30) shows that a monoclonal anti-D antibody leads to haemolysis and even anaemia concluding that this monoclonal anti-D antibody could not be used to treat autoimmune ITP.

In conclusion, there is a need for development of a safe and efficacious anti-D treatment of ITP without the disadvantage of WinRho® and Rhophylac®.

### SUMMARY OF INVENTION

The present invention relates to treatment of thrombocytopenia with the recombinant polyclonal anti-RhesusD antibody product such as the product disclosed in PCT/DK2005/000501 (Sym001).

Evidence in the literature suggests that the natural polyclonality of existing anti-D products is required for reliable efficacy, and accordingly the Sym001 composition has been selected to reflect the natural diversity of anti-D antibodies observed in the donor population. At the same time a recombinant polyclonal anti-RhesusD antibody product like Sym001 represents a limited number of antibodies, all of the IgG1 subclass, which can be characterized in accordance with the requirements for a well-defined biological product during and after production. Hence, a recombinant polyclonal anti-RhesusD antibody product like Sym001 can be characterized to a substantially higher degree than existing plasma-derived anti-D. A recombinant polyclonal anti-RhesusD antibody product such as Sym001 will also have a more reproducible composition compared to the lot-to-lot variation that may be found for a plasma-derived product, where the antibody repertoire varies depending on the repertoire present in the individual plasma donors. For a recombinant polyclonal anti-RhesusD antibody product like Sym001 the production strategy using recombinant DNA technology ensures that the same antibodies are produced each time, without the presence of any irrelevant immunoglobulin molecules.

A benefit to patients of introducing the recombinant polyclonal anti-D antibody product disclosed in the present invention, relates to the decreased risk of transmitting human pathogens when using a recombinant product as compared to a blood-based product and a more favourable supply situation. The more specific anti-RhD activity may result in a better risk/benefit profile than that of blood derived products.

Another benefit is that a recombinant polyclonal anti-RhesusD antibody product like Sym001 will not lead extra vascular haemolysis and/or a significant decreased haemoglobin level. Accordingly, a recombinant polyclonal anti-RhesusD antibody product like Sym001 can be used for treatment of ITP without consideration of the haemoglobin level of the patient before or after the treatment. This results in a safe and efficacious treatment of ITP.

The present invention concerns a recombinant polyclonal anti-RhesusD antibody product for use in the treatment or prophylaxis of thrombocytopenia, wherein said antibody product is prepared for administration in a dose of 10-500 microgram specific antibody/kg patient body mass, said recombinant polyclonal anti-RhesusD antibody product comprising a defined subset of individual antibodies, exhibiting binding to at least one epitope on the Rhesus D antigen.

The present invention further relates to use of recombinant polyclonal anti-RhesusD antibody product in the manufacture of a medicament for the treatment or prophylaxis of thrombocytopenia, wherein said antibody is prepared for administration in a dose of 10-500 microgram specific antibody/kg patient body mass.

A further aspect of the invention relates to a method of treatment of thrombocytopenia in a subject, said method comprising administering to said subject suffering from thrombocytopenia a therapeutically effective amount of a recombinant anti-RhesusD antibody product, wherein said antibody is administered in a dose of 10-500 microgram specific antibody/kg patient body mass. The invention also relates to treatment of a subject suffering from thrombocytopenia which also has anaemia.

The anti-RhesusD antibody product can in one embodiment be administered intravenously or subcutaneously.

The present invention further relates to a method of avoiding extravascular haemolysis during anti-RhesusD based treatment in a subject suffering from thrombocytopenia, said method comprising administering to a subject suffering from thrombocytopenia a therapeutically effective amount of a recombinant anti-RhesusD antibody, wherein said antibody is administered in a dose of 10-500 microgram specific antibody/kg patient body mass.

Another aspect of the invention relates to a composition for treatment of thrombocytopenia comprising the anti-RhesusD antibody product and a physiologically acceptable carrier and/or a pharmaceutically acceptable carrier.

The present invention also relates to a kit-of-parts comprising the anti-RhesusD antibody product and at least one additional component.

### DEFINITIONS AND ABBREVIATIONS

The term “antibody” describes a functional component of serum and is often referred to either as a collection of molecules (antibodies or immunoglobulin) or as one molecule (the antibody molecule or immunoglobulin molecule). An antibody molecule is capable of binding to or reacting with a specific antigenic determinant (the antigen or the antigenic epitope), which in turn may lead to induction of immunological effector mechanisms. An individual antibody molecule is usually regarded as monospecific, and a composition of antibody molecules may be monoclonal (i.e., consisting of identical antibody molecules) or polyclonal (i.e., consisting of different antibody molecules reacting with the same or different epitopes on the same antigen or even on distinct, different antigens). Each antibody molecule has a unique structure that enables it to bind specifically to its corresponding antigen, and all natural antibody molecules have the same overall basic structure of two identical light chains and two identical heavy chains. Antibodies are also known collectively as immunoglobulins. The terms antibody or antibodies as used herein are also intended to include chimeric and single chain antibodies, as well as binding fragments of antibodies, such as Fab, Fab′ or F(ab)₂ molecules, Fv fragments or scFv fragments or any other stable fragment, as well as full-length antibody molecules and multimeric forms such as dimeric IgA molecules or pentavalent IgM.

The term “anti-RhD antibody-encoding nucleic acid segment” describes a nucleic acid segment comprising a pair of V_{H} and V_{L} genetic elements. The segment may further comprise light chain and/or heavy chain constant region genetic elements, e.g. Kappa or Lambda light chain constant region and/or one or more of the constant region domains CH1, CH2, CH3 or CH4 selected from one of the isotypes IgG1, IgG2, IgG3, IgG4, IgA1, IgA2, IgM, IgD and IgE. The preferred isotypes are IgG1 and/or IgG3. The nucleic acid segment may also comprise one or more promoter cassettes, either facilitating bi-directional or uni-directional transcription of the V_{H} and V_{L}-encoding sequences. Additional transcriptional or translational elements, such as functional leader sequences directing the gene product to the secretory pathway, poly A signal sequences, UCOE's and/or an IRES may also be present in the segment.

The term “anti-RhD recombinant polyclonal antibody” or “anti-RhD rpAb” describes a composition of recombinantly produced diverse antibody molecules, where the individual members are capable of binding to at least one epitope on the Rhesus D antigen.

Preferably, the composition is produced from a single manufacturing cell line. The diversity of the polyclonal antibody is located in the variable regions (V_{H} and V_{L} regions), in particular in the CDR1, CDR2 and CDR 3 regions.

The terms “a distinct member of the anti-RhD rpAb” denotes an individual antibody molecule of the recombinant polyclonal antibody composition, comprising one or more stretches within the variable regions, which are characterized by differences in the amino acid sequence compared to the other individual members of the polyclonal protein. These stretches are in particular located in the CDR1, CDR2 and CDR 3 regions.

The term “immunoglobulin” commonly is used as a collective designation of the mixture of antibodies found in blood or serum, but may also be used to designate a mixture of antibodies derived from other sources or is used in the term “immunoglobulin molecule”.

The term “polyclonal antibody” describes a composition of different (diverse) antibody molecules which is capable of binding to or reacting with several different specific antigenic determinants on the same or on different antigens. Usually, the variability of a polyclonal antibody is located in the so-called variable regions of the polyclonal antibody, in particular in the CDR regions. When stating that a member of a polyclonal antibody binds to an antigen, it is herein meant a binding having binding constant that is below 1 mM, preferably below 100 nM, even more preferred below 10 nM.

The term “recombinant antibody” is used to describe an antibody molecule or several molecules that is/are expressed from a cell or cell line transfected with an expression vector comprising the coding sequence of the protein which is not naturally associated with the cell.

If the antibody molecules are diverse or different, the term “recombinant polyclonal antibody” applies in accordance with the definition of a polyclonal antibody.

The following style of writing “V_{H}:LC” and “V_{H}:V_{L}” indicate a particular pair of a variable heavy chain sequence with a light chain or a variable light chain sequence. Such particular pairs of V_{H} and V_{L} sequences can either be nucleic acid sequences or polypeptides. In the present invention particular V_{H} and V_{L} pairs confer binding specificity towards the rhesus D antigen.

The term “RhesusD” also refer to RhesusD variants.

Abbreviations: Anti-RhD rpAb=anti-Rhesus D recombinant polyclonal antibody. CASY=Cell Counter+Analyzer System. ELISA=Enzyme-Linked Immunosorbent Assay. ITP=idiopathic thrombocytopenic purpura. pWCP=polyclonal working cell pool. RBC=red blood cells. RhD=Rhesus D. RhD(−)=Rhesus D negative. RhD(+)=Rhesus D positive. RhD^{VI}=Rhesus D category VI antigen. Anti-D=polyclonal immunoglobulin preparation against RhD from hyperimmune donors.

### FIGURE LEGENDS

FIG. 1A-C: Alignment of the nucleic acid sequences encoding the variable heavy chain (V_{H}) of the 56 selected RhD clones. The individual clone names are indicated to the right of the alignment, and the position of CDR regions are indicated above the alignments.

FIG. 2A-E: Alignment of the nucleic acid sequences encoding the entire light chain of the 56 selected RhD clones. The individual clone names together with an indication of whether it is a Kappa or Lambda chain are indicated to the right of the alignment, and the position of CDR regions are indicated above the alignments.

FIG. 3: Alignment of the amino acid sequences corresponding to V_{H} of the 56 selected RhD clones. The individual clone names are indicated to the right of the alignment, and the position of CDR regions are indicated above the alignments.

FIG. 4A-B: Alignment of the amino acid sequences corresponding to V_{L} of the 56 selected RhD clones, wherein (A) corresponds to the Kappa chains and (B) to the Lambda chains. The individual clone names are indicated to the right of the alignment, and the position of CDR regions are indicated above the alignments.

FIG. 5: Cation exchange chromatograms of anti-RhD rpAb composition from aliquots 3948 and 3949 after 9 weeks cultivation. The lower diagram corresponds to aliquot 3949 and the upper one to aliquot 3948. The Y-axis of the top diagram has been displaced in order to separate it from the lower diagram. Peaks A-J comprise antibodies differing in net charge and individual antibodies appearing charge heterogeneous.

FIG. 6: Gel picture showing HinfI RFLP analysis on RT-PCR product derived from polyclonal cell line aliquots 3948+ and 3949+ (FCWO65) producing anti-RhD rpAb after 11 weeks cultivation. Bands which can be assigned to specific clones are identified.

FIG. 7: (A) Shows a comparison of the potency of three batches, Sym04:21, Sym04:23, and Sym04:24, of anti-RhD pAb with 25 individual members, produced by fed batch cultivation in 5 L scale. Binding of pAb to RhD-positive erythrocytes was measured by FACS and the mean fluorescence intensity (MFI) is shown as a function of pAb concentration in ng/ml. Further, the functional activity of an anti-RhD pAb with 25 individual members was measured on Sym04:21 and Sym04:24 in a combined ADCC/phagocytosis assay. (B) Shows the ADCC results as percentage of specific lysis of RhD-positive and RhD-negative erythrocytes as a function of pAb concentration in ng/ml. (c) Shows the percentage of phagocytosis of RhD-positive and RhD-negative erythrocytes as a function of pAb concentration in ng/ml.

FIG. 8: Comparability of Sym001 and the plasmaderived anti-D product Winrho using functional in vitro assays. The percentage of A: PBMC mediated ADCC of RhD positive or negative RBCs, B: PBMC mediated phagocytosis of RhD positive or negative RBCs and C: THP-1 cell line mediated phagocytosis of opsonized platelets as a function of concentration of anti-D antibodies in Sym001 or WinRho. The individual measurement is based on triplicates. The standard deviations are indicated by bars.

FIG. 9: FIG. 9 shows changes in mean Hemoglobin level between baseline and different time-points post-dose in Rhesus D positive subjects dosed with Sym001 at different dose levels or with placebo. At none of the dose levels the changes relative to baseline were statistically significant or clinically important at any time point post dose. The greatest mean fall in Hemoglobin was observed in the 25 ug/kg group at day 21 and was 0.42 g/dL, and the greatest mean fall in the 75 ug/kg group was observed at day 14 and day 21 and was 0.3 g/dL

### DETAILED DESCRIPTION OF THE INVENTION

### Thrombocytopenia

The present invention relates to treatment of Thrombocytopenia with recombinant polyclonal anti-RhesusD antibody product. Thrombocytopenia (or -paenia, or thrombopenia in short) is the presence of relatively few platelets in blood. The treatment of Thrombocytopenia with the recombinant polyclonal anti-RhesusD antibody product can in one embodiment be symptomatic and/or ameliorating and/or prophylactic and/or curative.

In humans, a normal platelet count ranges from 150,000 and 450,000 per mm³ (microlitre). These limits, however, are determined by the 2.5th lower and upper percentile, and a deviation does not necessarily imply any form of disease. In one embodiment the present invention relates to treatment of Thrombocytopenia with recombinant polyclonal anti-RhesusD antibody product of an individual, such as a human being, with platelet count below 150,000 per mm³ (microlitre), such as below 140,000 per mm³, for example below 130,000 per mm³, such as below 120,000 per mm³, for example below 110,000 per mm³, such as below 100,000 per mm³, for example below 80,000 per mm³, such as below 60,000 per mm³, for example below 40,000 per mm³ or such as below 20,000 per mm³.

### Diagnosis of Thrombocytopenia

Thrombocytopenia can be diagnosed by different laboratory tests might include the following measurements: a full blood count, measurement of liver enzymes, measurement of renal function, measurement of vitamin B12 levels, folic acid levels, erythrocyte sedimentation rate, and/or peripheral blood smear. If the cause for the low platelet count remains unclear, bone marrow biopsy is often undertaken, to differentiate whether the low platelet count is due to decreased production or peripheral destruction.

The present invention relates to treatment of Thrombocytopenia with recombinant polyclonal anti-RhesusD antibody product which has been diagnosed by any method including the ones listed herein above.

### Causes of Thrombocytopenia

Decreased platelet counts can be due to a number of disease processes including the ones mentioned herein below. The present invention relates to treatment of any type of Thrombocytopenia with recombinant polyclonal anti-RhesusD antibody product. The cause of Thrombocytopenia can be, but is not limited to, one or more of the causes listed herein below.

A) Decreased production of platelets caused by one or more of the factors listed below:
vitamin B12 or folic acid deficiency
leukemia or myelodysplastic syndrome
decreased production of thrombopoietin by the liver in liver failure.
sepsis, systemic viral or bacterial infection
Dengue fever can cause thrombocytopenia by direct infection of bone marrow
megakaryocytes as well as immunological shortened platelet survival
Hereditary syndromes
Congenital Amegakaryocytic Thrombocytopenia (CAMT)
Thrombocytopenia absent radius syndrome
Fanconi anemia
Bernard-Soulier syndrome, associated with large platelets
May-Hegglin anomaly, the combination of thrombocytopenia, pale-blue leuckocyte inclusions, and giant platelets
Grey platelet syndrome
Alport syndrome

B) Increased destruction of platelets caused by one or more factors listed below:
hemolytic-uremic syndrome (HUS)
disseminated intravascular coagulation (DIC)
paroxysmal nocturnal hemoglobinuria (PNH)
antiphospholipid syndrome
systemic lupus erythematosus (SLE)
post transfusion purpura
neonatal alloimmune thrombocytopenia (NAITP)
Splenic sequestration of platelets due to hypersplenism
Dengue fever has been shown to cause shortened platelet survival and immunological platelet destruction
HIV

C) Medication-induced Thrombocytopenia
Heparin
Valproic acid
Quinidine
Abciximab
Sulfonamide antibiotics
Interferons
Measles-mumps-rubella vaccine
Glycoprotein IIb/IIIa inhibitors
Clopidogrel
Vancomycin
Linezolid
Famotidine
Mebeverine
Tinidazole/Metronidazole
drugs for direct myelosuppression such as Valproic acid, Methotrexate, Carboplatin, Interferon and other chemotherapy drugs.
drugs for Immunological platelet destruction such as drugs that bind Fab portion of an antibody (e.g. quinidine group of drugs), drug that bind to Fc, and drug-antibody complex that bind and activate platelets.

### Treatment of Thrombocytopenia

Treatment is guided by etiology and disease severity. The main concept in treating thrombocytopenia is to eliminate the underlying problem, whether that means discontinuing suspected drugs that cause thrombocytopenia, or treating underlying sepsis.

The present invention relates to treatment of any type of Thrombocytopenia with recombinant polyclonal anti-RhesusD antibody product. The treatment of Thrombocytopenia with recombinant polyclonal anti-RhesusD antibody product can in one embodiment be combined with one or more other treatments of Thrombocytopenia including one or more of the treatments listed herein below.

Thrombotic Thrombocytopenic Purpura (TTP)

Treatment of TTP was revolutionized in the 1980s with the application of plasmapheresis. According to the Furlan-Tsai hypothesis, this treatment theoretically works by removing antibodies directed against the von Willebrand factor cleaving protease, ADAMTS-13. The plasmapheresis procedure also adds active ADAMTS-13 protease proteins to the patient, restoring a more physiological state of von Willebrand factor multimers.

Idiopathic Thrombocytopenic Purpura (ITP)

Treatments for ITP include prednisone and other corticosteroids, Intravenous gamma globulin, Splenectomy, Danazol, Rituximab, Thrombopoetin analogues and AMG 531 (Romiplostim, trade name Nplate).

### Therapeutic Compositions

In an embodiment of the invention, a pharmaceutical composition comprising the anti-RhesusD antibody product is intended for treatment of thrombocytopenia.

In one embodiment the pharmaceutical composition further comprises a pharmaceutically acceptable excipient.

The anti-RhesusD antibody product may be administered within a pharmaceutically-acceptable diluent, carrier, or excipient, in unit dosage form. Conventional pharmaceutical practice may be employed to provide suitable formulations or compositions to administer to patients. In a preferred embodiment the administration is prophylactic.

Any appropriate route of administration may be employed, for example, administration may be parenteral, intravenous, intra-arterial, subcutaneous, intramuscular, intraperitoneal, intranasal, aerosol, suppository, or oral administration.

For example, therapeutic formulations may be in the form of, liquid solutions or suspensions; for oral administration, formulations may be in the form of tablets or capsules chewing gum or pasta, and for intranasal formulations, in the form of powders, nasal drops, or aerosols.

The pharmaceutical compositions of the present invention are prepared in a manner known per se, for example, by means of conventional dissolving, lyophilizing, mixing, granulating or confectioning processes. The pharmaceutical compositions may be formulated according to conventional pharmaceutical practice (see for example, in Remington: The Science and Practice of Pharmacy (20th ed.), ed. A. R. Gennaro, 2000, Lippincott Williams & Wilkins, Philadelphia, Pa. and Encyclopedia of Pharmaceutical Technology, eds. J. Swarbrick and J. C. Boylan, 1988-1999, Marcel Dekker, New York, N.Y.).

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, pills, or capsules, which may be coated with shellac, sugar or both. It is also possible for them to be incorporated into plastics carriers that allow the active ingredients to diffuse or be released in measured amounts. For oral administration the pharmaceutical composition can be protected to prevent digestion of said composition in the gastric acid in the stomach.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, tablets, pills, or capsules. The formulations can be administered to human individuals in therapeutically or prophylactic effective amounts (e.g., amounts which prevent, eliminate, or reduce a pathological condition) to provide therapy for a disease or condition. The preferred dosage of therapeutic agent to be administered is likely to depend on such variables as the type and extent of the disorder, the overall health status of the particular patient, the formulation of the compound excipients, and its route of administration.

Treatment of Thrombocytopenia with recombinant polyclonal anti-RhesusD antibody product is in one preferred embodiment prepared for administration in a dose of 10-500 microgram specific antibody/kg patient body mass per dose, such as from 10-25 microgram specific antibody/kg patient body mass, for example from 25-50 microgram specific antibody/kg patient body mass, such as from 50-75 microgram specific antibody/kg patient body mass, for example from 75-100 microgram specific antibody/kg patient body mass, such as from 100-125 microgram specific antibody/kg patient body mass, for example from 125-150 microgram specific antibody/kg patient body mass, such as from 150-175 microgram specific antibody/kg patient body mass, for example from 175-200 microgram specific antibody/kg patient body mass, such as from 200-225 microgram specific antibody/kg patient body mass, for example from 225-250 microgram specific antibody/kg patient body mass, such as from 250-275 microgram specific antibody/kg patient body mass, for example from 275-300 microgram specific antibody/kg patient body mass, such as from 300-325 microgram specific antibody/kg patient body mass, for example from 325-350 microgram specific antibody/kg patient body mass, such as from 350-375 microgram specific antibody/kg patient body mass, for example from 375-400 microgram specific antibody/kg patient body mass, such as from 400-425 microgram specific antibody/kg patient body mass, for example from 425-450 microgram specific antibody/kg patient body mass, such as from 450-475 microgram specific antibody/kg patient body mass or for example from 475-500 microgram specific antibody/kg patient body mass.

In one embodiment treatment of Thrombocytopenia in splenectomised patients or Rhesus negative patients with recombinant polyclonal anti-RhesusD antibody product comprises administration of the recombinant polyclonal anti-RhesusD antibody product in a dose of 10-2000 microgram specific antibody/kg patient body mass per dose, such as from 10-25 microgram specific antibody/kg patient body mass, for example from 25-50 microgram specific antibody/kg patient body mass, such as from 50-75 microgram specific antibody/kg patient body mass, for example from 75-100 microgram specific antibody/kg patient body mass, such as from 100-125 microgram specific antibody/kg patient body mass, for example from 125-150 microgram specific antibody/kg patient body mass, such as from 150-175 microgram specific antibody/kg patient body mass, for example from 175-200 microgram specific antibody/kg patient body mass, such as from 200-225 microgram specific antibody/kg patient body mass, for example from 225-250 microgram specific antibody/kg patient body mass, such as from 250-275 microgram specific antibody/kg patient body mass, for example from 275-300 microgram specific antibody/kg patient body mass, such as from 300-325 microgram specific antibody/kg patient body mass, for example from 325-350 microgram specific antibody/kg patient body mass, such as from 350-375 microgram specific antibody/kg patient body mass, for example from 375-400 microgram specific antibody/kg patient body mass, such as from 400-425 microgram specific antibody/kg patient body mass, for example from 425-450 microgram specific antibody/kg patient body mass, such as from 450-475 microgram specific antibody/kg patient body mass, for example from 475-500 microgram specific antibody/kg patient body mass, such as from 500-550 microgram specific antibody/kg patient body mass, for example from 550-600 microgram specific antibody/kg patient body mass, such as from 600-650 microgram specific antibody/kg patient body mass, for example from 650-700 microgram specific antibody/kg patient body mass, such as from 700-750 microgram specific antibody/kg patient body mass, for example from 750-800 microgram specific antibody/kg patient body mass, such as from 800-850 microgram specific antibody/kg patient body mass, for example from 850-900 microgram specific antibody/kg patient body mass, such as from 900-950 microgram specific antibody/kg patient body mass, for example from 950-1000 microgram specific antibody/kg patient body mass, such as from 1000-1050 microgram specific antibody/kg patient body mass, for example from 1050-1100 microgram specific antibody/kg patient body mass, such as from 1100-1150 microgram specific antibody/kg patient body mass, for example from 1150-1200 microgram specific antibody/kg patient body mass, such as from 1200-1250 microgram specific antibody/kg patient body mass, for example from 1250-1300 microgram specific antibody/kg patient body mass, such as from 1300-1350 microgram specific antibody/kg patient body mass, for example from 1350-1400 microgram specific antibody/kg patient body mass, such as from 1400-1450 microgram specific antibody/kg patient body mass, for example from 1450-1500 microgram specific antibody/kg patient body mass, such as from 1500-1550 microgram specific antibody/kg patient body mass, for example from 1550-1600 microgram specific antibody/kg patient body mass, such as from 1600-1650 microgram specific antibody/kg patient body mass, for example from 1650-1700 microgram specific antibody/kg patient body mass, such as from 1700-1750 microgram specific antibody/kg patient body mass, for example from 1750-1800 microgram specific antibody/kg patient body mass such as from 1800-1850 microgram specific antibody/kg patient body mass, for example from 1850-1900 microgram specific antibody/kg patient body mass, such as from 1900-1950 microgram specific antibody/kg patient body mass, or for example from 1950-2000 microgram specific antibody/kg patient body mass.

### Therapeutic Uses of the Compositions According to the Invention

The pharmaceutical compositions according to the present invention may be used for the treatment, amelioration or prophylaxis of Thrombocytopenia in a mammal such as a human being.

One aspect of the present invention is a method for disease treatment, amelioration or prophylaxis in an animal or a human being, wherein an effective amount of recombinant polyclonal anti-RhesusD antibody product is administered.

The pharmaceutical compositions according to the present invention can in one embodiment be administered once, once per day, repeatedly with one or more days intervals such as 2 days, three days, four days, five days, six days, 7 days or repeatedly once or twice per week, or once or twice per month or once or twice per year.

### Hemoglobin Levels

The present invention further relates to treatment of thrombocytopenia in an individual such as an anemic human being. Anemia or anaemia is defined as a qualitative or quantitative deficiency of hemoglobin, a molecule found inside red blood cells. The hemoglobin level depends on the age and gender of the individual.

In one embodiment of the invention the haemoglobin level for the subject is less than 15 g/dL, such as less than 14 g/dL, for example less than 13 g/dL, such as less than 12 g/dL, for example less than 11 g/dL, such as less than 10 g/dL, for example less than 9 g/dL, such as less than 8 g/dL.

Anaemia can be defined as a Haemoglobin pre-dose value lower than 2.0 g/dL below the lower limit of the laboratory normal range for gender and age. Gender can be divided into the groups male and female. Age can be divided into the groups, newborn, children and adults. A subgroup of adults comprises pregnant adult females.

An alternative definition of anaemia is a haemoglobin level of 2 Standard deviations (SD) below normal laboratory range for age and sex. 2 SD would approximately correspond to 2 g/dL.

The standard diagnosis of anemia in adults corresponds to hemoglobin values <12 g/dL in women and <14 g/dL in men and are based on a reference from WHO: World Health Organization: Nutritional Anemia: Report of a WHO Scientific Group. Geneva: World Health Organization, 1968.

Normal haemoglobin values depend on the individual laboratory standards but are approximately as follows (source: http://www.medical-library.net/content/view/297/41/):

Adult males: 13-18 g/dL haemoglobin
Adult females: 12-16 g/dL haemoglobin
Pregnant females: 11-12 g/dL haemoglobin
Newborn: 17-19 g/dL haemoglobin (77% of this value is fetal hemoglobin, which drops to approximately 23% of the total at 4 months of age).
Children: 14-17 g/dL haemoglobin.

### Recombinant Polyclonal Anti-RhesusD Antibody Product

Recombinant polyclonal anti-RhesusD antibody products for use in the present invention has been disclosed in PCT/DK2005/000501 and is also described herein below.

In a further embodiment of the present invention, an anti-RhD recombinant polyclonal antibody composition comprises a defined subset of individual antibodies, based on the common feature that they exhibit binding to at least one epitope on the Rhesus D antigen e.g. epD1, epD2, epD3, epD4, epD5, epD6/7, epD8 and/or epD9, but not or very weakly to Rhesus C, c, E, e antigens. Preferably the anti-RhD rpAb composition is composed of at least one antibody which bind to epD3, epD4 and epD9 (RhD category VI antigen binding antibody) and further antibodies which at least in combination binds to the remaining epitopes epD1, epD2, epD5, epD6/7 and epD8, e.g. an antibody against RhD category II or III antigen, or a RhD category IV or V antigen binding antibody combined with an antibody against category VII antigen. Typically an anti-RhD rpAb composition has at least 5, 10, 20, 50, 100 or 500 distinct variant members. The preferred number of variant members range between 5 and 100, even more preferred between 5 and 50 and most preferred between 10 and 30 such as between 10 and 25.

In addition to the variability of the V_{H} and V_{L} regions, in particular the CDR regions, the constant regions may also be varied with respect to isotype. This implies that one particular V_{H} and V_{L} pair may be produced with varying constant heavy chain isotypes, e.g. the human IgG1, IgG2, IgG3, IgG4, IgA1, IgA2, IgM, IgD and IgE. Thus, an anti-RhD rpAb may comprise antibody molecules that are characterized by sequence differences between the individual antibody molecules in the variable region (V region) as well as in the constant region. The anti-RhD rpAb composition can be composed of antibodies with any heavy chain isotype mentioned above or combinations thereof. Preferred anti-RhD rpAb compositions contain IgG1 constant regions, IgG3 constant regions or IgG1 and IgG3 constant regions. In a preferred embodiment of the present invention each or some of the V_{H} and V_{L} pairs are expressed with a human IgG1, IgG3, IgA1 and/or IgA2 constant heavy chain.

In order to provide a library of anti-RhD antibody-encoding nucleic acid segments a number of methods known in the art may be utilized. A first library comprising V_{H} and V_{L}-encoding segments may either be generated by combinatorial techniques (e.g. EP 0 368 684) or techniques maintaining the cognate pairing (pairs of variable region-encoding sequences derived from the same cell, described in WO 05/042774). Further, V_{H} and V_{L}-encoding segment libraries may be generated by incorporating isolated CDR gene fragments, into an appropriate framework (e.g. Soderlind, E. et al., 2000. Nat. Biotechnol. 18, 852-856), or by mutation of one or more anti-RhD V_{H} and V_{L}-encoding sequences. This first library is screened for V_{H} and V_{L}-encoding nucleic acid segments producing antibodies or fragments with binding specificity towards RhD, thereby generating a library of anti-RhD Ab-encoding nucleic acid segments. In particular with combinatorial libraries the screening is preceded by an enrichment step for example a so-called biopanning step. Known biopanning technologies are phage display (Kang, A. S. et al. 1991. Proc Natl Acad Sci USA 88, 4363-4366), ribosome display (Schaffitzel, C. et al. 1999. J. Immunol. Methods 231, 119-135), DNA display (Cull, M. G. et al. 1992. Proc Natl Acad Sci USA 89, 1865-1869), RNA-peptide display (Roberts, R. W., Szostak, J. W., 1997. Proc Natl Acad Sci USA 94, 12297-12302), covalent display (WO 98/37186), bacterial surface display (Fuchs, P. et al. 1991. Biotechnology 9, 1369-1372), yeast surface display (Boder, E. T., Wittrup, K. D., 1997. Nat Biotechnol 15, 553-557) and eukaryotic virus display (Grabherr, R., Ernst, W., 2001. Comb. Chem. High Throughput. Screen. 4, 185-192). FACS and magnetic bead sorting are also applicable for enrichment (panning) purposes using labeled antigen. The screening for Rhesus D binders are generally performed with immunodetection assays such as agglutination, FACS, ELISA, FLISA and/or immunodot assays.

Following screening, the generated sub-library of V_{H} and V_{L}-encoding nucleic acid segments, generally needs to be transferred from the screening vector to an expression vectors suitable for site-specific integration and expression in the desired host cell. It is important that the sequences encoding the individual V_{H}:V_{L} pairs are maintained during the transfer. This can either be achieved by having the individual members of the sub-library separate and moving V_{H} and V_{L}-encoding sequences one by one. Alternatively, the vectors constituting the sub-library are pooled, and the sequences encoding the V_{H}:V_{L} pairs are moved as segments, keeping the V_{H} and V_{L}-encoding sequences together during the transfer. This process is also termed mass transfer, and enables an easy transfer of all the selected V_{H}:V_{L} pairs from one vector to another.

The anti-RhesusD antibody product preferably comprises antibodies with reactivities against D+ and all variants tested (DIII, DIV, DV, DVI type I-III, DVII, DFR, RoHAR, DOL, DAR, DHMi, DBT, Weak D type 1, 2, 3, 4 and 12).

The present invention relates to an Anti-RhesusD antibody product that comprises antibodies with the CDR sequences of the 25 antibodies encoded by clones RhD157, 159, 160, 162, 189, 191, 192, 196, 197, 199, 201, 202, 203, 207, 240, 241, 245, 293, 301, 305, 306, 317, 319, 321, and 324.

The present invention also relates to an Anti-RhesusD antibody product that comprises antibodies with the V_{H}V_{L} sequences of the 25 antibodies encoded by clones RhD157, 159, 160, 162, 189, 191, 192, 196, 197, 199, 201, 202, 203, 207, 240, 241, 245, 293, 301, 305, 306, 317, 319, 321, and 324.

In one preferred embodiment the present invention relates to an Anti-RhesusD antibody product that comprises the antibodies encoded by clones RhD157, 159, 160, 162, 189, 191, 192, 196, 197, 199, 201, 202, 203, 207, 240, 241, 245, 293, 301, 305, 306, 317, 319, 321, and 324.

In one preferred embodiment the Anti-RhesusD antibody product is manufactured in mammalian cell, more preferably it is manufactured with the glycosylation obtainable by expression in CHO cells.

Structural Characterization of Anti-RhD rpAb

Structural characterization of polyclonal antibodies requires high resolution due to the complexity of the mixture (clonal diversity, heterogeneity and glycosylation). Traditional approaches such as gel filtration, ion-exchange chromatography or electrophoresis may not have sufficient resolution to differentiate among the individual antibodies in the anti-RhD rpAb. Two-dimensional polyacrylamide gel electrophoresis (2D-PAGE) has been used for profiling of complex protein mixtures followed by mass spectrometry (MS) or liquid chromatography (LC)-MS (e.g., proteomics). 2D-PAGE, which combines separation on the basis of a protein's charge and mass, has proven useful for differentiating among polyclonal, oligoclonal and monoclonal immunoglobulin in serum samples. However, this method has some limitations. Chromatographic techniques, in particular capillary and LC coupled to electrospray ionization MS are increasingly being applied for the analysis of complex peptide mixtures. LC-MS has been used for the characterization of monoclonal antibodies and recently also for profiling of polyclonal antibody light chains. The analysis of very complex samples requires more resolving power of the chromatographic system, which can be obtained by separation in two dimensions (or more). Such an approach is based on ion-exchange in the first dimension and reversed-phase chromatography (or hydrophobic interaction) in the second dimension optionally coupled to MS.

Functional Characterization of Anti-RhD rpAb

An anti-RhD rpAb antibody can for example be characterized functionally through comparability studies with anti-D immunoglobulin products or anti-RhD mAbs. Such studies can be performed in vitro as well as in vivo.

In vitro functional characterization methods of anti-RhD rpAb could for example be phagocytosis assays (⁵¹Cr-based or FACS based), antibody dependent cellular cytotoxicity (ADCC) and rosetting assay. Briefly described the assays are performed as follows:

### ADCC Assay (⁵¹Cr Based):

Human PBMC are used as effector cells and RhD negative and positive RBC (0 in the AB0 system) are used as targets. First, the RBC (RhD(+) and RhD(−)) are ⁵¹Cr labelled, washed and then sensitized with anti-RhD antibodies (e.g. anti-RhD rpAb, anti-D or anti-RhD mAb) in various dilutions. The effector cells (PMBC) are added to the sensitized RBC (ratio of 20:1) and incubation is performed overnight. Cells are spun down and the supernatants from the wells are transferred to a Lumaplate (PerkinElmer). Controls for spontaneous release are included (RBC with ⁵¹Cr only) and for total release (addition of Triton-X-100 to ⁵¹Cr-labeled RBC). The Lumaplate is dried and counted in a Topcounter (PerkinElmer).

### Phagocytosis Assay (⁵¹Cr Based):

Phagocytosis can be measured in combination with the ADCC assay. After harvesting the supernatant in the ADCC assay, the remaining supernatant is removed and the red blood cells are lysed by addition of a hypotonic buffer. The cells are washed and the supernatant is removed. PBS+1% Triton-X-100 is added to all wells and fixed amounts are transferred to a Lumaplate, dried and counted as before.

### Phagocytosis Assay (FACS Based):

This assay is based on adherence of the phagocytic cells. The human leukemic monoblast cell line U937 can be used for this assay. U937 cells are differentiated using 10 nM PMA. Two days later 60% of the medium is removed and replaced by medium without PMA. The cell membrane of red blood cells (RhD(+) and RhD(−)) are stained with PKH26 (PE) according to the manufactures protocol (Sigma). The RBC's are sensitized with anti-RhD antibodies in various dilutions and excess antibodies are removed by washing. On day three, the non-adherent cells U937 cell are removed by washing and sensitized RBC (RhD(+) and RhD(−)) are added to the wells. The plates are incubated overnight in the incubator. Non-phagocytozed RBC are washed away by several steps. Attached but not phagocytozed RBC are lysed by addition of hypotonic buffer followed by additional washing. The U937 cells detached from the wells by incubation with trypsin. Cells are analyzed on the FACS.

### Assay for Determination of Inhibition of Platelet Phagocytosis:

This functional assay determines the dose dependent inhibition of platelet phagocytosis. Breifly, platelets are labeled with CM green and opsonized with antibodies and incubated with effector cells: THP-1a monocytic cell line. Platelets, red blood cells and THP-1 cells were prepared as described in Example 4. Phagocytosis of platelets were determined as follows. Platelets (2×10⁸/ml 50 μl/well for 1:20 E:T ratio), RBC (4×10⁸/ml 50 μl/well for 1:40 E:T ratio) and THP-cells (1×10⁷/ml 50 μl/well) were mixed and incubated 2 h in a humified incubator (5% CO₂— 37° C.). 100 μl Trypan Blue, Fluka prediluted 1:1 in PBS was added to block the nonspecific binding on the outside of THP-cells. Washed once in 200 μl/well PBS (210 g +4° C., 3 min), 200 μl/well Lysing solution, BD was added and incubated 15 min at 4° C. Washed once in 200 μl/well PBS and resuspended in 200 μl/well PBS. Cells were acquired live gate thru SSC and FSC on HTS on FACS Calibur and the Median fluorescence intensity of FI-1 was analyzed.

### Rosetting Assay

A rosetting assay is merely an Fc receptor binding assay. Sensitized red blood cells are incubated with differentiated U937 cells prepared as described above. RBC (RhD(−) and RhD(+)) are sensitized with anti-RhD antibodies in various dilutions and excess antibodies are removed by washing before they are mixed with U937 cells. Incubation is performed for one hour and non-bound RBC are washed away. The percentage of cells with two or more RBC attached to the surface is counted.

An in vivo functional characterization of anti-RhD antibodies is described by Miescher (Miescher, S., et al. 2004, Blood 103, 4028-4035), an involves injection of RhD(+) cells into RhD(−) individuals followed by administration of anti-RhD antibody. RBC clearance and anti-RhD antibody sensation of the donors was analyzed.

### Production of the Recombinant Polyclonal Anti-RhesusD Antibody Product

### The Recombinant Polyclonal Protein Expression System

The present invention provides a recombinant polyclonal antibody expression system for the consistent manufacturing of anti-RhD recombinant polyclonal antibody (anti-RhD rpAb) from one or a few cell lines. Anti-RhD recombinant polyclonal antibody (anti-RhD rpAb) may be manufactured and/or purified and/or characterized as described in PCT/DK2005/000501.

In addition to the variability of the V_{H} and V_{L} regions, in particular the CDR regions, the constant regions may also be varied with respect to isotype. This implies that one particular V_{H} and V_{L} pair may be produced with varying constant heavy chain isotypes, e.g. the human IgG1, IgG2, IgG3, IgG4, IgA1, IgA2, IgM, IgD and IgE. Thus, an anti-RhD rpAb may comprise antibody molecules that are characterized by sequence differences between the individual antibody molecules in the variable region (V region) as well as in the constant region. The anti-RhD rpAb composition can be composed of antibodies with any heavy chain isotype mentioned above or combinations thereof. Preferred anti-RhD rpAb compositions contain IgG1 constant regions, IgG3 constant regions or IgG1 and IgG3 constant regions. In a preferred embodiment of the present invention each or some of the V_{H} and V_{L} pairs are expressed with a human IgG1, IgG3, IgA1 and/or IgA2 constant heavy chain.

In order to provide a library of anti-RhD antibody-encoding nucleic acid segments a number of methods known in the art may be utilized. A first library comprising V_{H} and V_{L}-encoding segments may either be generated by combinatorial techniques (e.g. EP 0 368 684) or techniques maintaining the cognate pairing (pairs of variable region-encoding sequences derived from the same cell, described in WO 05/042774). Further, V_{H} and V_{L}-encoding segment libraries may be generated by incorporating isolated CDR gene fragments, into an appropriate framework (e.g. Soderlind, E. et al., 2000. Nat. Biotechnol. 18, 852-856), or by mutation of one or more anti-RhD V_{H} and V_{L}-encoding sequences. This first library is screened for V_{H} and V_{L}-encoding nucleic acid segments producing antibodies or fragments with binding specificity towards RhD, thereby generating a library of anti-RhD Ab-encoding nucleic acid segments. In particular with combinatorial libraries the screening is preceded by an enrichment step for example a so-called biopanning step. Known biopanning technologies are phage display (Kang, A. S. et al. 1991. Proc Natl Acad Sci USA 88, 4363-4366), ribosome display (Schaffitzel, C. et al. 1999. J. Immunol. Methods 231, 119-135), DNA display (Cull, M. G. et al. 1992. Proc Natl Acad Sci USA 89, 1865-1869), RNA-peptide display (Roberts, R. W., Szostak, J. W., 1997. Proc Natl Acad Sci USA 94, 12297-12302), covalent display (WO 98/37186), bacterial surface display (Fuchs, P. et al. 1991. Biotechnology 9, 1369-1372), yeast surface display (Boder, E. T., Wittrup, K. D., 1997. Nat Biotechnol 15, 553-557) and eukaryotic virus display (Grabherr, R., Ernst, W., 2001. Comb. Chem. High Throughput. Screen. 4, 185-192). FACS and magnetic bead sorting are also applicable for enrichment (panning) purposes using labeled antigen. The screening for Rhesus D binders are generally performed with immunodetection assays such as agglutination, FACS, ELISA, FLISA and/or immunodot assays.

Following screening, the generated sub-library of V_{H} and V_{L}-encoding nucleic acid segments, generally needs to be transferred from the screening vector to an expression vectors suitable for site-specific integration and expression in the desired host cell. It is important that the sequences encoding the individual V_{H}:V_{L} pairs are maintained during the transfer. This can either be achieved by having the individual members of the sub-library separate and moving V_{H} and V_{L}-encoding sequences one by one. Alternatively, the vectors constituting the sub-library are pooled, and the sequences encoding the V_{H}:V_{L} pairs are moved as segments, keeping the V_{H} and V_{L}-encoding sequences together during the transfer. This process is also termed mass transfer, and enables an easy transfer of all the selected V_{H}:V_{L} pairs from one vector to another.

In a further embodiment of the present invention, an anti-RhD recombinant polyclonal antibody composition comprises a defined subset of individual antibodies, based on the common feature that they exhibit binding to at least one epitope on the Rhesus D antigen e.g. epD1, epD2, epD3, epD4, epD5, epD6/7, epD8 and/or epD9, but not or very weakly to Rhesus C, c, E, e antigens. Preferably the anti-RhD rpAb composition is composed of at least one antibody which bind to epD3, epD4 and epD9 (RhD category VI antigen binding antibody) and further antibodies which at least in combination binds to the remaining epitopes epD1, epD2, epD5, epD6/7 and epD8, e.g. an antibody against RhD category II or III antigen, or a RhD category IV or V antigen binding antibody combined with an antibody against category VII antigen. Typically an anti-RhD rpAb composition has at least 5, 10, 20, 50, 100 or 500 distinct variant members. The preferred number of variant members range between 5 and 100, even more preferred between 5 and 50 and most preferred between 1+ and 30 such as between 10 and 25.

A further embodiment of the present invention is a recombinant polyclonal manufacturing cell line, comprising a collection of cells transfected with a library of anti-RhD polyclonal antibody-encoding nucleic acid segments, wherein each cell in the collection is capable of expressing one member of the library, which encodes a distinct member of an anti-RhD rpAb or fragment and which is located at the same site in the genome of individual cells in said collection, wherein said nucleic acid segment is not naturally associated with said cell in the collection.

In an additional embodiment the variant nucleic acid segments encoding the anti-RhD rpAb are all derived from naturally occurring sequences, for example isolated from a donor, either as combinatorial V_{H}:V_{L} pairs or as cognate pairs, and not derived by mutation.

Compositions of cells that contain variant nucleic acids located at a single specific site in the genome within each cell have been described in WO 02/44361. This document discloses the use of the cells to identify molecules having desirable properties, but the reference does not deal with the provision of a production system or with the provision of polyclonal antibody characterized by a specific binding to an antigen.

### Clonal Diversity/Polyclonality

One of the characteristics of a polyclonal antibody is that it is constituted of a number of individual antibody molecules where each antibody molecule is homologous to the other molecules of the polyclonal antibody, but also has a variability that is characterized by differences in the amino acid sequence between the individual members of the polyclonal antibody. These differences are normally confined to the variable region in particular the CDR regions, CDR1, CDR2 and CDR3. This variability of a polyclonal antibody can also be described as diversity on the functional level, e.g., different specificity and affinity with respect to different antigenic determinants on the same or different antigens located on one or more targets. In a recombinant polyclonal antibody the diversity constitutes a sub-set of the diversity observed in a donor derived immunoglobulin product. Such a sub-set is carefully selected and characterized with respect to its ability to bind desired target antigens, in this particular case the Rhesus D antigen.

One of the concerns with respect to production of a recombinant polyclonal antibody may be whether the clonal diversity is maintained in the final product. The clonal diversity may be analyzed by RFLP or sequencing of (RT)-PCR products from the cells expressing the anti-RhD rpAb. The diversity can also be analyzed on protein level by functional tests (e.g., ELISA) on the anti-RhD rpAb produced by the cell line, by anti-idiotypic antibodies to individual members or by chromatographic methods.

Clonal bias, if it exists, can be estimated by comparing the clonal diversity of the initial library, used for transfection, with the diversity found in the pool of cells (polyclonal cell line) expressing the anti-RhD rpAb.

Clonal diversity of an anti-RhD rpAb can be assessed as the distribution of individual members of the polyclonal composition. This distribution can be assessed as the total number of different individual members in the final polyclonal antibody composition compared to the number of different encoding sequences originally introduced into the cell line during transfection. In this case sufficient diversity is considered to be acquired when at least 50% of the encoding sequences originally used in the transfection can be identified as different individual members of the final anti-RhD rpAb. Preferably at least 75% of the anti-RhD antibody-encoding sequences used for transfection can be identified as antibodies in the final composition. Even more preferred at least 85% to 95%, and most preferred a 100% of the anti-RhD antibody-encoding sequences used for transfection can be identified as antibodies in the final composition.

The distribution of individual members of the anti-RhD rpAb composition can also be assessed with respect to the mutual distribution among the individual members. In this case sufficient clonal diversity is considered to be acquired if no single member of the composition constitutes more than 75% of the total number of individual members in the final anti-RhD rpAb composition. Preferably, no individual member exceeds more that 50%, even more preferred 25% and most preferred 10% of the total number of individual members in the final polyclonal composition. The assessment of clonal diversity based on the distribution of the individual members in the polyclonal composition can be performed by RFLP analysis, sequence analysis or protein analysis such as the approaches described later on for characterization of a polyclonal composition.

Clonal diversity may be reduced as a result of clonal bias which can arise a) during the cloning process, b) as a result of variations in cellular proliferation, or c) through scrambling of multiple integrants. If such biases arise, each of these sources of a loss of clonal diversity is easily remedied by minor modifications to the methods as described herein. In order to limit bias introduced by cloning of the variable domains into the appropriate vectors, the transfer of the genes of interest from one vector to another may be designed in such a way that cloning bias is limited. Mass transfer techniques and a careful selection of the E. coli strain used for amplification can reduce the cloning bias. Another possibility is to perform an individual transfer of each polynucleotide encoding an individual member of the polyclonal antibody, between screening vectors and vectors for site-specific integration. It is possible that variations in cellular proliferation rates of the individual cells in the cell line could, over a prolonged period of time, introduce a bias into the anti-RhD rpAb expression, increasing or reducing the presence of some members of the anti-RhD rpAb expressed by the cell line. One reason for such variations in proliferation rates could be that the population of cells constituting the starting cell line used for the initial transfection is heterogeneous. It is known that individual cells in a cell line develop differently over a prolonged period of time. To ensure a more homogeneous starting material, sub-cloning of the cell line prior to transfection with the library of interest may be performed using a limiting dilution of the cell line down to the single cell level and growing each single cell to a new population of cells (so-called cellular sub-cloning by limiting dilution). One or more of these populations of cells are then selected as starting material based on their proliferation and expression properties. Further, the selection pressure used to ensure that only cells that have received site-specific integrants will survive, might affect proliferation rates of individual cells within a polyclonal cell line. This might be due to the favoring of cells that undergo certain genetic changes in order to adapt to the selection pressure. Thus, the choice of selection marker might also influence proliferation rate-induced bias. If this occurs, different selection markers should be tested. In cases where selection is based on a substance that is toxic to the cells, the optimal concentration should be tested carefully, as well as whether selection is needed throughout the entire production period or only in the initial phase.

An additional approach to ensure a well defined cell population is to use fluorescence activated cell sorting (FACS) after the transfection and selection procedures. Fluorescence labeled antibodies can be used to enrich for highly productive cells derived from a pool of cells transfected with IgG constructs (Brezinsky et al. J. 2003. Immunol Methods 277, 141-155). This method can also be used to sort cells expressing similar levels of immunoglobulin, thereby creating a homogenous cell population with respect to productivity. Likewise, by using labeling with the fluorescent dye 5,6-carboxylfluorescein diacetate succinimidyl ester (CFSE) cells showing similar proliferation rates can be selected by FACS methods. Further, differences in expression levels of the individual members of the anti-RhD rpAb may also introduce a bias into the final product over a prolonged period of time. If the polyclonal cell line is generated by mixing separately transfected clones after selection, the following selection criteria may be set up for the individual clones at the cell culture level prior to mixing: proliferation rates have to be between 24 and 32 hours, the productivity should exceed 1.5 pg antibody per cell per day, and the culture should show a homogenous cell population assessed by an intra cellular staining method. If desired a more homogenous cell population for each individual clone can be obtained with the surface staining method described by Brezinsky prior to mixing the individual clones by gating on a particular area of the population in connection with the FACS analysis.

Even if a proliferation rate-induced, or productivity-induced bias occurs, the loss or over-representation of individual members might not necessarily be critical, depending on the diversity requirements of the final anti-RhD rpAb product.

In cells with site-specific single integrants, the cells will only differ in the sequence of the variable regions of the antibodies to be expressed. Therefore, the different cellular effects imposed by variation in integration site and gene regulatory elements are eliminated and the integrated segments have minimal effects on the cellular proliferation rate. Neither scrambling nor multiple integrations is likely to cause problems in the proliferation rate of the manufacturing cell line, since these are rare events. Random integrations generally occur with an efficiency of approximately 10⁻⁵, whereas site-specific integration occurs with an efficiency of approximately 10⁻³. If multiple integrations should unexpectedly pose a problem, an alternative is to repeat the transfection with the library of anti-RhD antibody expression vectors, because the likelihood that the event will reoccur is very small, as described above. Considering statistics, bulk transfection of a large number of cells also constitutes a way to circumvent an undesired clonal bias. In this approach, a host cell line is transfected in bulk with the library of anti-RhD antibody expression vectors. Such a library constitutes many copies of each distinct member of the library. These copies should preferably be integrated into a large number of host cells. Preferably at least 100, 1000, 10000 or 100000 individual cells are transfected with copies of distinct members of the library of variant nucleic acid segments. Thus, if a library of distinct variant nucleic acid segments is composed of 1000 distinct members which are each integrated into 1000 individual cells, 10⁶ clones containing a site-specifically integrated anti-RhD antibody-encoding segment should arise from the transfection. In this manner the gausian curve of individual cell doubling rates should influence the general population only in very small degrees. This will increase the probability of keeping the clonal composition constant, even if a low percentage of the manufacturing cells should exhibit aberrant growth and/or expression properties.

Alternatively the semi-bulk transfection or individual transfection methods previously described may be used.

### EXAMPLES

### Example 1

### Production of an Anti-Rhesus D Recombinant Polyclonal Antibody

### Donors

Donors were enrolled at Aalborg Sygehus Nord. A total of eight RhD(−) women were immunized with RhD(+) erythrocytes derived from RhD(+) individuals. The donors had a varying history of the immunizations with respect to the number of boosts and the origin of RhD(+) erythrocytes for the immunization. The immunization history of the different donors is given in the table 1.

TABLE 1

| Donor | # of | # of boosts from |
|---|---|---|
| # | boost | different origin |
| 1 | 3 | 2 |
| 2 | 6 | 2 |
| 3 | 2 | 1 |
| 4 | 4 | 4 |
| 5 | 2 | 2 |
| 6 | 3 | 2 |
| 7 | 2 | 2 |
| 8 | 2 | 2 |

Mononuclear cells were harvested by leukopheresis 5-7 days after the last boost. The cells were pelleted and immediately transferred to the cell lysis solution from a commercially available RNA preparation kit (NucleoSpin RNA L, Machery-Nagel, cat. no. 740 962.20). After lysis of the cells, the suspension was frozen before further processing.

### Generation of Anti-Rhesus D Fab Display Library

The material obtained from each donor was kept separate throughout the procedure of library generation and panning. The cell lysates were thawed and RNA was prepared according to kit instructions (NucleoSpin RNA L). The integrity of the RNA was analyzed by agarose gel electrophoresis, thus verifying that the 18S/28S ribosomal RNAs were not degraded.

RNA was subjected to cDNA synthesis in an oligo(dT) primed reaction using approximately 10 μg total RNA in a reaction using ThermoScript (Invitrogen), according to the manufacturer's instructions. The cDNA was used as template in PCR reactions using the following primers:

### V_{H} Forward Primers (XhoI Site in Bold):

| J region | SEQ ID | Primer sequenoe |
|---|---|---|
| JH1/2 | 2 | GGAGGCGCTC GAGACGGTGA CCAGGGTGCC |
| JH3 | 3 | GGAGGCGCTC GAGACGGTGA CCATTGTCCC |
| JH4/5 | 4 | GGAGGCGCTC GAGACGGTGA CCAGGGTTCC |
| JH6 | 5 | GGAGGCGCTC GAGACGGTGA CCGTGGTCCC |

### V_{H} Reverse Primers (AscI Site in Bold):

| Vgene | | |
|---|---|---|
| family | SEQ ID | Primer sequence |
| 1B/7A | 6 | CCAGCCGGGG CGCGCCCAGR TGCAGCTGGT |
| | | GCARTCTGG |
| 1C | 7 | CCAGCCGGGG CGCGCCSAGG TCCAGCTGGT |
| | | RCAGTCTGG |
| 2B | 8 | CCAGCCGGGG CGCGCCCAGR TCACCTTGAA |
| | | GGAGTCTGG |
| 3B | 9 | CCAGCCGGGG CGCGCCSAGG TGCAGCTGGT |
| | | GGAGTCTGG |
| 3C | 10 | CCAGCCGGGG CGCGCCGAGG TGCAGCTGGT |
| | | GGAGWCYGG |
| 4B | 11 | CCAGCCGGGG CGCGCCCAGG TGCAGCTACA |
| | | GCAGTGGGG |
| 4C | 12 | CCAGCCGGGG CGCGCCCAGS TGCAGCTGCA |
| | | GGAGTCSGG |
| 5B | 13 | CCAGCCGGGG CGCGCCGARG TGCAGCTGGT |
| | | GCAGTCTGG |
| 6A | 14 | CCAGCCGGGG CGCGCCCAGG TACAGCTGCA |
| | | GCAGTCAGG |

### C_{κ} Forward Primer (NotI Site in Bold):

| SEQ ID | Primer sequence |
|---|---|
| 15 | ACCGCCTCCA CCGGCGGCCG CTTATTAACA CTCTCCCCTG |
| | TTGAAGCTCT T |

V_{κ} reverse Primers (NheI Site in Bold):

| V gene | | |
|---|---|---|
| family | SEQ IDP | rimer sequence |
| 1B | 16 | CAACCAGCGC TAGCCGACAT CCAGWTGACC |
| | | CAGTCTCC |
| 2 | 17 | CAACCAGCGC TAGCCGATGT TGTGATGACT |
| | | CAGTCTCC |
| 3B | 18 | CAACCAGCGC TAGCCGAAAT TGTGWTGACR |
| | | CAGTCTCC |
| 4B | 19 | CAACCAGCGC TAGCCGATAT TGTGATGACC |
| | | CACACTCC |
| 5 | 20 | CAACCAGCGC TAGCCGAAAC GACACTCACG |
| | | CAGTCTCC |
| 6 | 21 | CAACCAGCGC TAGCCGAAAT TGTGCTGACT |
| | | CAGTCTCC |

### C_{λ} Forward Primer (NotI Sit in Bold):

| λ | SEQ | |
|---|---|---|
| family | ID | Primer sequence |
| 2 | 22 | ACCGCCTCCACCGGCGGCCGCTTATTATGAACATTCTGTA |
| | | GGGCCACTG |
| 7 | 23 | ACCGCCTCCACCGGCGGCCGCTTATTAAGAGCATTCTGCA |
| | | GGGGCCACTG |

### V_{λ} Reverse Primers (NheI in Bold):

| V gene | | |
|---|---|---|
| family | SEQID | Primer sequence |
| 1A | 24 | CAACCAGCGC TAGCCCAGTC TGTGCTGACT |
| | | CAGCCACC |
| 1B | 25 | CAACCAGCGC TAGCCCAGTC TGTGYTGACG |
| | | CAGCCGCC |
| 1C | 26 | CAACCAGCGC TAGCCCAGTC TGTCGTGACG |
| | | CAGCCGCC |
| 2 | 27 | CAACCAGCGC TAGCCCARTC TGCCCTGACT |
| | | CAGCCT |
| 3A | 28 | CAACCAGCGC TAGCCCTTTC CTATGWGCTG |
| | | ACTCAGCCACC |
| 3B | 29 | CAACCAGCGC TAGCCCTTTC TTCTGAGCTG |
| | | ACTCAGGACCC |
| 4 | 30 | CAACCAGCGC TAGCCCACGT TATACTGACT |
| | | CAACCGCC |
| 5 | 31 | CAACCAGCGC TAGCCCAGGC TGTGCTGACT |
| | | CAGCCGTC |
| 6 | 32 | CAACCAGCGC TAGCCCTTAA TTTTATGCTG |
| | | ACTCAGCCCCA |
| 7/8 | 33 | CAACCAGCGC TAGCCCAGRC TGTGGTGACY |
| | | CAGGAGCC |
| 9 | 34 | CAACCAGCGC TAGCCCWGCC TGTGCTGACT |
| | | CAGCCMCC |

PCR was performed with individual primer pairs amounting to 36 V_{H} reactions, 6 Kappa reactions and 22 Lambda reactions. All V_{H}, Kappa, and Lambda PCR products were pooled separately and following purification using NucleoSpin columns (Machery-Nagel, cat. no. 740 590.250), the products were digested prior to cloning (V_{H}: AscI/XhoI, Kappa and Lambda: NheI/NotI) followed by a gel purification step of the bands of interest (PerfectPrep Gel Cleanup kit, Eppendorf, cat. no. 0032 007.759). The light chains (Kappa and Lambda separately) were inserted into a NheI/NotI treated Em351 phage display vector, by ligation and amplified in E. coli XL1 Blue (Stratagene). Plasmid DNA constituting the light chain library was isolated from the E. coli cells selected over night on Carbenicillin agar plates (two libraries for each donor, Kappa and Lambda, respectively). This library DNA was subjected to digest with AscI/XhoI, and after gel purification, the V_{H} PCR products (subjected to digest with the same enzymes and gel purified) were ligated into the two light chain libraries from each donor and amplified in E. coli TG1 cells (Stratagene) using Carbenicillin selection on agar plates. After overnight growth, bacteria were scraped off the plates, and glycerol stocks were prepared for proper library storage. A plasmid DNA preparation containing the combinatorial variable heavy chain—light chain (V_{H}:LC) library was also performed to secure the library for the future. The combinatorial libraries contained in the TG1 cells (two from each donor) were now ready for phage display and panning. The sizes of the combinatorial libraries (16 in total) were 10⁶ or larger.

### Enrichment for Phages Displaying Rhesus D Antigen Binding Fab Fragments

Phages displaying Fabs on their surface were generated as follows: 50 mL 2×YT/1% glucose/100 μg/mL Carbenicillin was inoculated with TG1 cells containing the combinatorial V_{H}:V_{L} library to obtain an OD₆₀₀ of approximately 0.08. The culture was shaking for 1½ h, and helper phage was added (VSCM13). The culture was incubated at 37° C. for ½ h without shaking and for ½ h with shaking. The bacteria were pelleted (3200×g, 10 minutes, 4° C.), and re-suspended in 50 mL 2×YT/100 μg/mL Carbenicillin/70 μg/mL Kanamycin, and the culture was shaken overnight at 30° C. The phages were precipitated from the culture supernatant by adding ⅕ volume of 20% PEG/1.5 M NaCl, incubating on ice for 30 minutes, and centrifugation at 8000×g for 30 minutes at 4° C. Precipitated phages were resuspended in PBS and used directly for panning.

Panning for Rhesus D antigen binding Fab fragments was performed in a two-step procedure. 10⁸ RhD(−) red blood cells (RBC) were washed three times in PBS (centrifugation at 2000×g, 45 sec), and re-suspended in 150 μl panning buffer (2% skim milk in 0.85×PBS). Fifty μl freshly prepared phages were added to the RhD(−) cells (re-suspended in panning buffer) in order to perform a negative selection step, and incubated for 1 h on an end-over-end rotator at 4° C. Following the one hour incubation, the cells were pelleted by centrifugation (2000×g, 45 sec), and the phage-containing supernatant was incubated with 2×10⁷ RhD(+) RBC (washed three times in PBS). The phage:RhD(+) RBC mix was incubated for one hour on an end-over-end rotator at 4° C. Unbound phages were removed by washing five times with 1 mL panning buffer, and five times with PBS. Bound phages were eluted by addition of 200 μl H₂O (which lyses the cells). One hundred μl of the eluate was added to exponentially growing TG1 cells, the remainder was stored at −80° C. TG1 cells infected with eluated phages were plated on Carb/glu agar dishes and incubated overnight at 37° C. The following day, the colonies were scraped off the plates, and 10 mL culture medium was inoculated for preparation of phages for the second round of panning. The second round of panning was performed as described for the first round.

### Enrichment for Phages Displaying Rhesus D Category VI Antigen Binding Fab Fragments

In a separate set of pannings, selections were performed in order to retrieve clones with reactivity towards the RhD category VI antigen. The negative selection was performed on RhD(−) blood as described, and the positive selection was performed on RhD^{VI} positive erythrocytes. The procedure was otherwise as described above.

### Screening for Anti-RhD Binding Fabs

After each round of panning single colonies were picked for analysis of their binding properties to red blood cells in agglutination assays. Briefly, single colonies were inoculated into 2×YT/100 μg/mL carbenicillin/1% glucose and shaken overnight at 37° C. The next day, DeepWell plates were inoculated using 900 μl 2×YT/100 μg/mL carbenicillin/0.1% glucose and 10 μl overnight culture. The plates were shaken for two hours at 37° C., before Fab induction was performed with addition of 300 μl 2×YT/100 μg/mL carbenicillin/0.25 mM IPTG per well. The plate was shaken overnight at 30° C. The following day, the bacteria were pelleted by centrifugation (3200×g, 4° C., 10 minutes), and re-suspended in 100 μl of 0.8 M NaCl, 0.2×PBS, 8 mM EDTA, and incubated for 15 minutes on ice in order to perform a periplasmic extraction of the Fab fragments. The plate was transferred to −20° C. and finally the suspension was thawed and centrifugation was performed for 10 minutes at 4° C. and 3200×g. The periplasmic extract was used in ELISA assays for analysis of Fab content and in agglutination assays to evaluate the binding potential of the individual Fab fragments. The agglutination assay was performed as follows: RhD(−) and RhD(+) RBC were mixed in a 1:1 ratio, and washed 3 times in PBS. After the final wash, the cell mix was re-suspended in 1% BSA in PBS at a density of 1% cells, 50 μl was added to each well of a 96-plate. Periplasmic extracts were added to the wells. As a positive control Rhesogamma P immunoglobulin (Aventis) was used according to the manufacturer's instructions. The plates were incubated for one hour at room temperature with gentle shaking. The cells were washed three times with PBS, before the secondary antibody was added (goat anti-human Fab/FITC conjugate, Sigma F5512) in a 1:100 dilution. The plates were left for agglutination for one hour at room temperature without shaking. Fab fragments positive in the agglutination assay was determined by visual inspection, and recorded by taking a picture. Quantization of the binding activity of the Fab fragments was performed by FACS analysis of the agglutination samples.

When performing screening for clones with reactivity towards RhD^{VI}+ erythrocytes, such cells were used in conjunction with RhD(−) cells in a procedure otherwise identical to that described above.

### Selection of Diverse Anti-RhD Fab-Encoding Sequences

A total of 1700 RhD antigen binding clones were identified. All the positive clones were submitted for DNA sequencing. From these 56 clones were selected based on their unique set of heavy chain CDR sequences. For multiple clones which used the same heavy chain with different light chains, the clone which showed the highest binding activity in the FACS assay was selected. Thereby a sub-library comprised of pairs of variable heavy chain (V_{H}) and light chain (LC)-encoding sequences, representing a broad diversity with high RhD antigen specificity, was selected from all the positive clones.

The binding activity of these 56 clones was re-confirmed in agglutination assays, to ensure no false positive clones were selected.

The selected clones were further analyzed with respect to mutations due to for instance inter-family cross-priming, since such mutations may lead to overall structural changes of the expressed antibody possibly creating new epitopes and thereby result in an increased immunogenicity of the final product. Clones with such mutations were repaired as described in the following section relating to V_{H}:LC transfer from the phagemid vector to the mammalian expression vector.

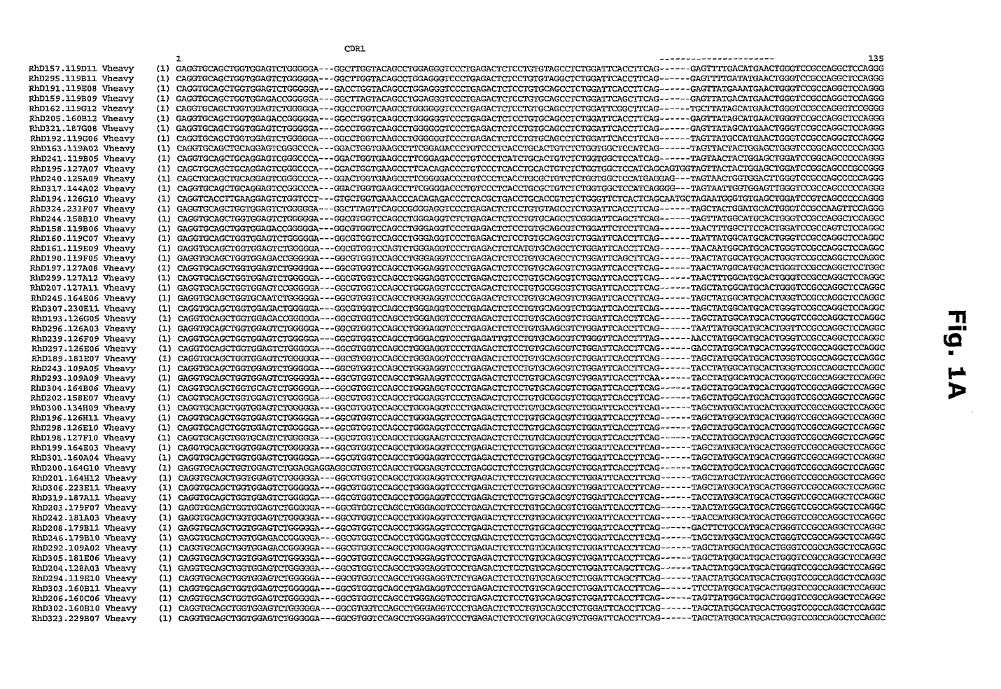

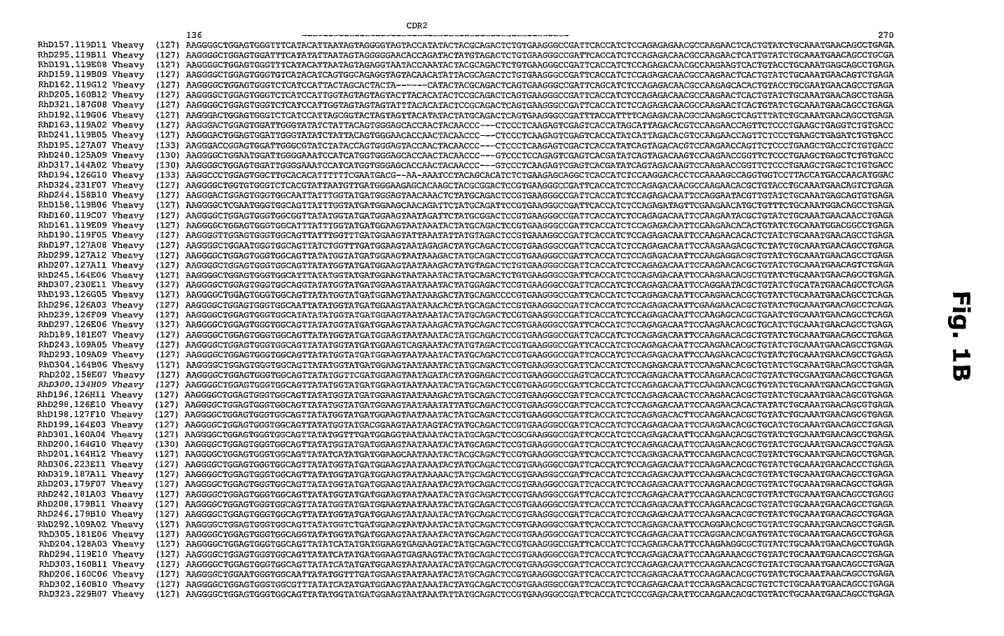

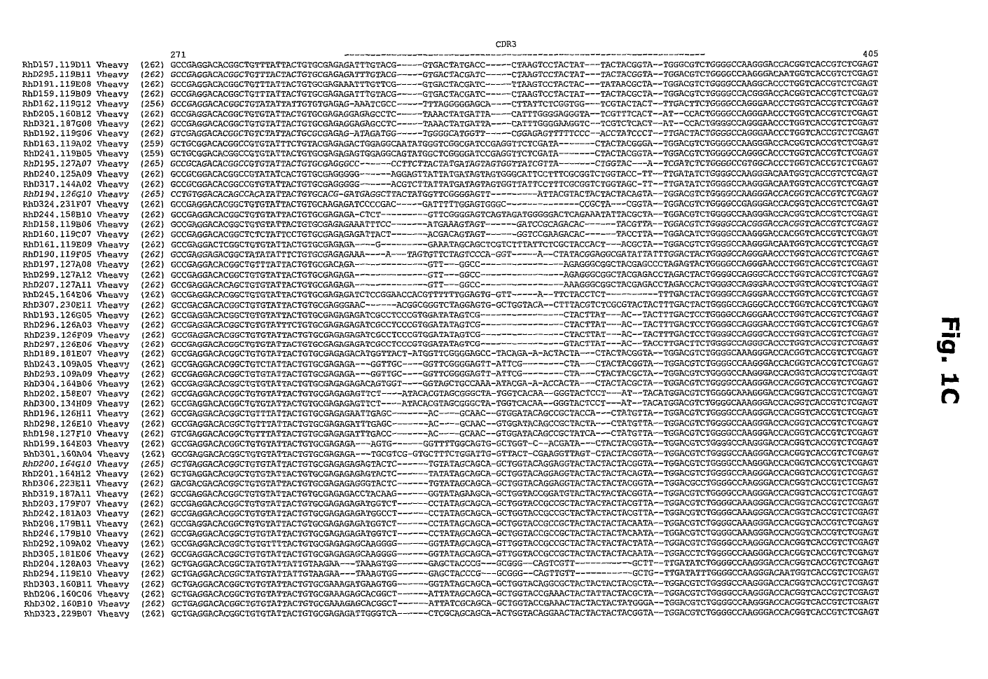

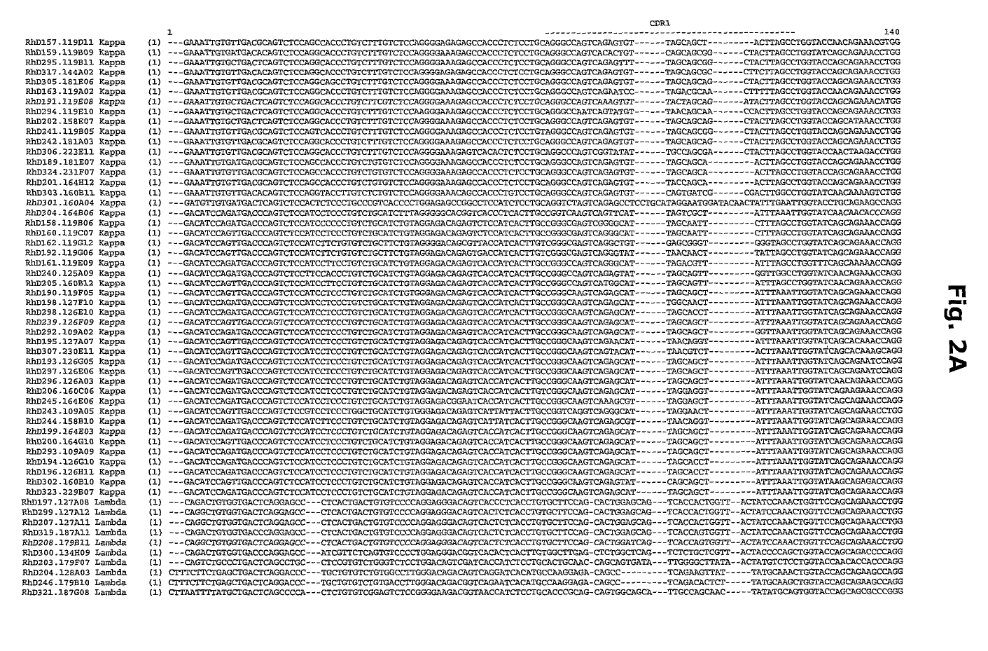

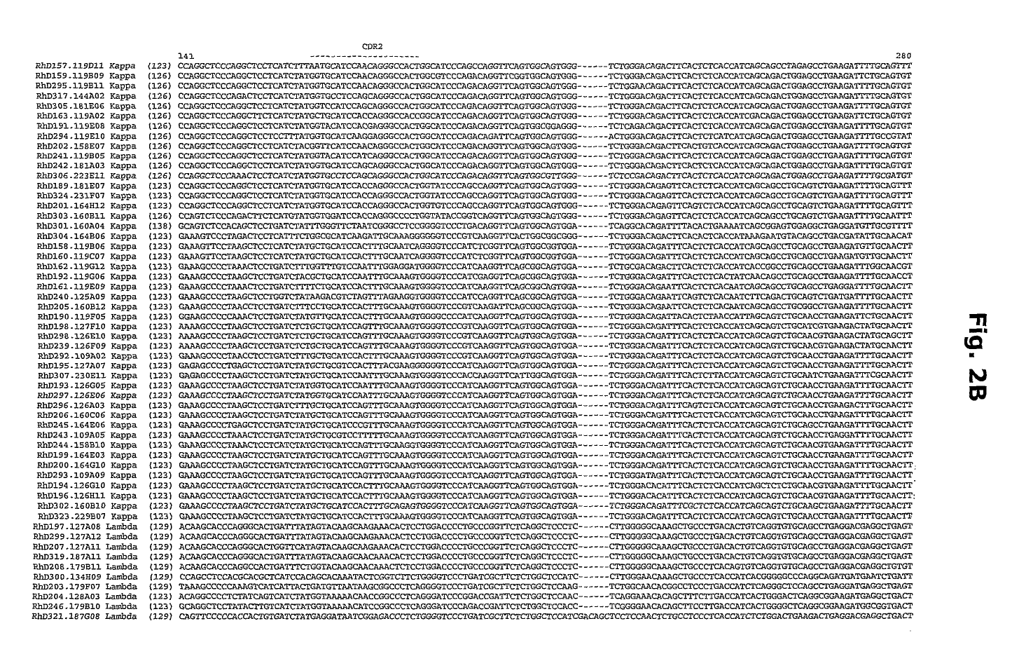

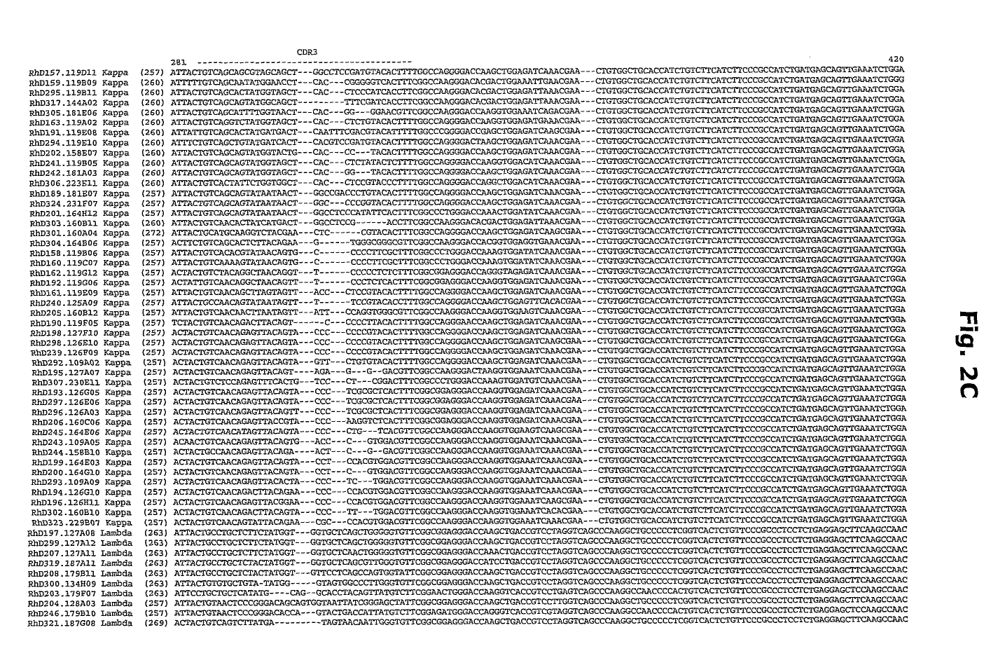

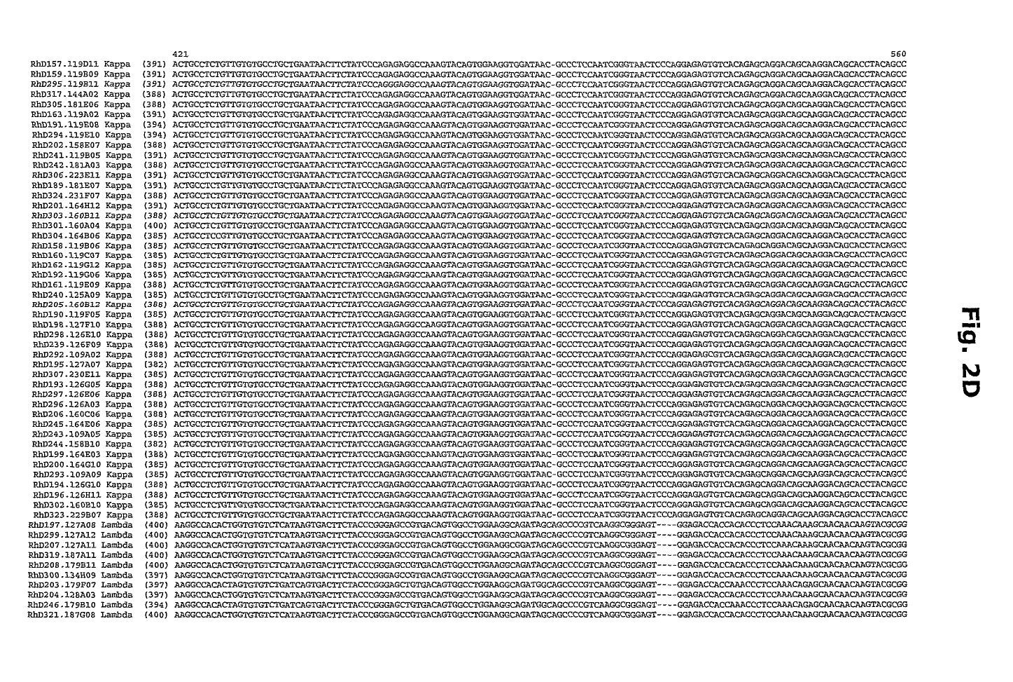

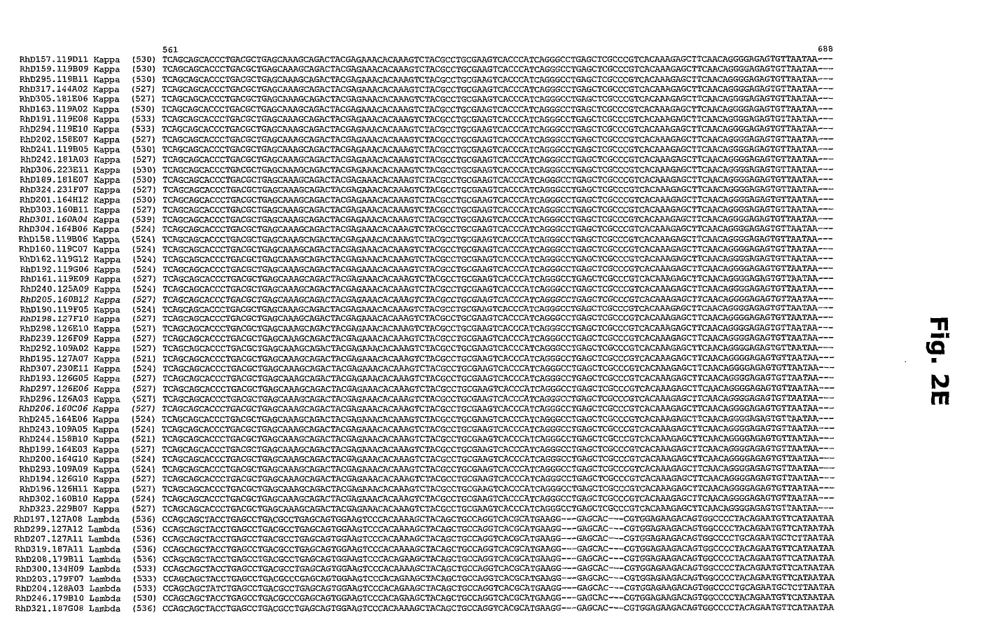

Alignments of the corrected nucleic acid sequences for the V_{H} and light chains (LC) are shown in FIGS. 1 to 4, respectively. Further alignments of the V_{H} and V_{L} polypeptide chains are shown in FIGS. 3 and 4, respectively. The polypeptide alignments were performed and numbered according to structural criteria defined by Chothia (Chothia et al. 1992 J. Mol. Biol. 227, 776-798; Tomlinson et al. 1995 EMBO J. 14, 4628-4638 and Williams et al. 1996 J. Mol. Biol., 264, 220-232). The figures further indicate the position of the three CDR regions within the variable regions. The CDR region positions within the amino acid sequences are summarized in table 2. The numbering of the CDR3 regions in the polypeptide alignments (FIGS. 3 and 4) does not follow Chothia (transition marked with an asterisk in the figures). In order to enable identification of the CDR3 region with respect to amino acid position, a continued numbering has been assigned after the asterisk. The CDR3 region sequence for each individual clone can be derived from the figures based on this numbering.

TABLE 2

| | V_{H} | V_{L} Kappa | V_{L} Lambda |
|---|---|---|---|
| | a.a. position | a.a. position | a.a. position |
| Figure | 3 | 4A | 4B |
| CDR1 | 31-35 | 24-34 | 25-35 |
| CDR2 | 50-65 | 50-56 | 53-57 |
| CDR3 | 95-125 | 89-110 | 90-113 |

The pairs of variable heavy chain and complete light chain which have been screened as Fabs and selected for their ability to bind RhD antigen can be identified by their identical clone numbers. All the 56 V_{H}:LC pairs are listed by clone number, the nucleic acid (nuc.) SEQ IDs and the amino acid (a.a.) SEQ IDs in table 3.

TABLE 3

| | V_{H} nuc. | LC nuc. | V_{H} a.a. | LC a.a |
|---|---|---|---|---|
| Clone Name | SEQ ID | SEQ ID | SEQ ID | SEQ ID |
| RhD157.119D11 | 35 | 91 | 147 | 203 |
| RhD158.119B06 | 36 | 92 | 148 | 204 |
| RhD159.119B09 | 37 | 93 | 149 | 205 |
| RhD160.119C07 | 38 | 94 | 150 | 206 |
| RhD161.119E09 | 39 | 95 | 151 | 207 |
| RhD162.119G12 | 40 | 96 | 152 | 208 |
| RhD163.119A02 | 41 | 97 | 153 | 209 |
| RhD189.181E07 | 42 | 98 | 154 | 210 |
| RhD190.119F05 | 43 | 99 | 155 | 211 |
| RhD191.119E08 | 44 | 100 | 156 | 212 |
| RhD192.119G06 | 45 | 101 | 157 | 213 |
| RhD193.126G05 | 46 | 102 | 158 | 214 |
| RhD194.126G10 | 47 | 103 | 159 | 215 |
| RhD195.127A07 | 48 | 104 | 160 | 216 |
| RhD196.126H11 | 49 | 105 | 161 | 217 |
| RhD197.127A08 | 50 | 106 | 162 | 218 |
| RhD198.127F10 | 51 | 107 | 163 | 219 |
| RhD199.164E03 | 52 | 108 | 164 | 220 |
| RhD200.164G10 | 53 | 109 | 165 | 221 |
| RhD201.164H12 | 54 | 110 | 166 | 222 |
| RhD202.158E07 | 55 | 111 | 167 | 223 |
| RhD203.179F07 | 56 | 112 | 168 | 224 |
| RhD204.128A03 | 57 | 113 | 169 | 225 |
| RhD205.160B12 | 58 | 114 | 170 | 226 |
| RhD206.160C06 | 59 | 115 | 171 | 227 |
| RhD207.127A11 | 60 | 116 | 172 | 228 |
| RhD208.179B11 | 61 | 117 | 173 | 229 |
| RhD239.126F09 | 62 | 118 | 174 | 230 |
| RhD240.125A09 | 63 | 119 | 175 | 231 |
| RhD241.119B05 | 64 | 120 | 176 | 232 |
| RhD242.181A03 | 65 | 121 | 177 | 233 |
| RhD243.109A05 | 66 | 122 | 178 | 234 |
| RhD244.158B10 | 67 | 123 | 179 | 235 |
| RhD245.164E06 | 68 | 124 | 180 | 236 |
| RhD246.179B10 | 69 | 125 | 181 | 237 |
| RhD292.109A02 | 70 | 126 | 182 | 238 |
| RhD293.109A09 | 71 | 127 | 183 | 239 |
| RhD294.119E10 | 72 | 128 | 184 | 240 |
| RhD295.119B11 | 73 | 129 | 185 | 241 |
| RhD296.126A03 | 74 | 130 | 186 | 242 |
| RhD297.126E06 | 75 | 131 | 187 | 243 |
| RhD298.126E10 | 76 | 132 | 188 | 244 |
| RhD299.127A12 | 77 | 133 | 189 | 245 |
| RhD300.134H09 | 78 | 134 | 190 | 246 |
| RhD301.160A04 | 79 | 135 | 191 | 247 |
| RhD302.160B10 | 80 | 136 | 192 | 248 |
| RhD303.160B11 | 81 | 137 | 193 | 249 |
| RhD304.164B06 | 82 | 138 | 194 | 250 |
| RhD305.181E06 | 83 | 139 | 195 | 251 |
| RhD306.223E11 | 84 | 140 | 196 | 252 |
| RhD307.230E11 | 85 | 141 | 197 | 253 |
| RhD317.144A02 | 86 | 142 | 198 | 254 |
| RhD319.187A11 | 87 | 143 | 199 | 255 |
| RhD321.187G08 | 88 | 144 | 200 | 256 |
| RhD323.229B07 | 89 | 145 | 201 | 257 |
| RhD324.231F07 | 90 | 146 | 202 | 258 |

### Transfer of the Selected V_{H} and Light Chain-Encoding Sequences to a Mammalian Expression Vector.

Due to the mutations resulting from, for instance, inter-family cross-priming it was necessary to repair of a large number of the selected sequences. This was done in connection with exchange of expression system from phage display to mammalian expression. For this reason the transfer was performed separately for each individual clone.

The transfer and repair was performed as follows: First the V_{H}-encoding sequence situated in the Em351 vector was re-amplified by PCR using the high fidelity polymerase, Phusion (Finnzymes) and a proper set of correcting primers. The V_{H} PCR fragment was digested with AscI and XhoI and subjected to gel purification. The Neo exp. vector was digested with the corresponding enzymes and gel purified thereby removing the nucleic acid sequence situated between the leader sequence and the heavy chain constant regions. The corrected V_{H} fragment and the Neo exp. vector were ligated and amplified in E. coli Top10 cells. Plasmid DNA of the V_{H} containing Neo exp. vector was isolated from the E. coli cells selected over night on Carbenicillin.

Following transfer of the V_{H}-encoding sequence the corresponding LC sequence was re-amplified by PCR using the high fidelity polymerase, Phusion (Finnzymes) and a proper set of correcting primers. The LC PCR fragment was digested with NheI and NotI and subjected to gel purification. The V_{H} containing Neo exp. vector was digested with the corresponding enzymes and gel purified thereby removing the nucleic acid sequence situated between the kappa leader sequence and the BGHpolyA signal sequence. The corrected LC fragment and the V_{H} containing Neo exp. vector were ligated and amplified in E. coli Top10 cells. Glycerol stocks were prepared for each individual clone, and a high quality plasmid preparation suitable for transfection of mammalian cells was prepared from the bacterial cultures as well. By performing the transfer separately for each clone the V_{H}:LC pairs originally selected by phage display were regenerated in the mammalian expression vector. In the instances where repair was not necessary the nucleic acid segment was transferred without performing PCR prior to the digestion with the appropriate restriction enzymes.

The mammalian expression vectors generated by the transfer described are suitable for expressing a full-length anti-RhD recombinant polyclonal antibody. Although the vectors are kept separate at this point it is still considered as a library of anti-RhD antibody expression vectors.

### Transfection and Selection of Mammalian Cell Lines

The Flp-In CHO cell line (Invitrogen) was used as starting cell line for establishment of a recombinant polyclonal manufacturing cell line. However, to obtain a more homogenous cell line the parental Flp-In CHO cell line was sub-cloned. Briefly, the parental cell line was sub-cloned by limited dilution and several clones were selected and expanded. Based on growth behavior one clone, CHO-Flp-In (019), was selected as production cell line. All the 56 plasmid preparations were transfected individually into the CHO-Flp-In (019) cell line as follows: the CHO-Flp-In (019) cells were cultured as adherent cells in F12-HAM with 10% fetal calf serum (FCS). 2.5×10⁶ cells were transfected with plasmid representing one clone using Fugene6 (Roche). Cells were trypsinated 24 hours after transfection and transferred to 3×T175 flasks. Selection pressure, in this case 450 μg/ml Neomycin, was added 48 hours after transfection. Approximately two weeks later clones appeared. Clones were counted and cells were trypsinated and hereafter cultured as pools of clones expressing one of the 56 specific anti-Rhesus-D antibodies.

### Adaptation to Serum Free Suspension Culture

The individual adherent anti-Rhesus-D antibody CHO-Flp-In (019) cell cultures were trypsinated, centrifuged and transferred to separate shaker flasks with 8×10⁵ cells/ml in appropriate serum free medium (Excell302, JRH Biosciences).

Growth and cell morphology were followed over several weeks. When cells showed good and stable growth behavior and had doubling time below 32 hours 50 aliquots of each culture with 10×10⁶ cells/tube were frozen down (56×50 aliquots).

### Characterization of Cell Lines

All the individual cell lines were characterized with respect to antibody production and proliferation. This was performed with the following assays:

### Production:

The production of recombinant antibodies in the individual cultures were followed over time by Kappa or Lambda specific ELISA. ELISA plates were coated overnight with goat-anti-human Kappa (Caltag) or goat-anti-human Lambda (Caltag) antibodies in carbonate buffer, pH 9.6. Plates were washed 6 times with washing buffer (1×PBS and 0.05% Tween 20) and blocked for 1 hour with washing buffer with 2% milk. Samples were added to wells and plates were incubated for 1 hour. Plates were washed 6× and secondary antibodies (goat-anti-human IgG (H+L) HRPO, Caltag) were added for 1 hour followed by 6× wash. ELISA was developed with TMB substrate and reaction stopped by addition of H₂SO₄. Plates were read at 450 nm.

Further, intracellular FACS staining, using fluorescently tagged antibodies was used to measure the production of recombinant antibodies in the cell culture system. 5×10⁵ cells were washed in cold FACS PBS (1×PBS ad 2% FCS) and centrifuged. Cells were fixed in CellFix (BD-Biosciences) for 20 min and hereafter washed in saponin buffer (1×PBS and 0.2% Saponin). The suspension was centrifuged and fluorescently tagged antibody (Goat F(ab′)₂ Fragment, Anti-human IgG(H+L)-PE, Beckman Coulter) was added for 20 min on ice. Cells were washed twice in saponin buffer and resuspended in FACS buffer and analyzed by FACS. This intracellular staining was used to determine the general expression level as well as to determine the homogeneity of the cell population in relation to expression of recombinant antibodies.

### Proliferation:

Aliquots of cell suspension were taken three times a week and cell number, cell size, degree of clumping and percentage of dead cells were determined by CASY® (Cell Counter+Analyzer System from Schärfe System GmbH) analysis. The doubling time for the cell cultures was calculated by cell number derived form CASY® measurements.

### Establishment of a Manufacturing Cell Line for Anti-Rhesus D Recombinant Polyclonal Antibody Production

Ten cell lines each expressing a distinct recombinant anti-Rhesus-D antibody (RhD157.119D11, RhD158.119B06, RhD159.119B09, RhD161.119E09, RhD163.119A02, RhD190.119F05, RhD191.119E08, RhD192.119G06, RhD197.127A08 and RhD204.128A03) were selected to constitute the recombinant polyclonal manufacturing cell line. The Rhd197 and RhD204 were lambda clones whereas all the others were kappa clones.

After the cell cultures expressing the individual anti-Rhesus antibodies were fully adapted to serum free suspension culture in shaker flasks they were mixed in equal cell number, thereby generating a polyclonal CHO-Flp-In (019) cell line. The mixed cell culture was centrifuged and frozen down in aliquots of 10×10⁶ cells/tube.

Two tubes (3948 FCWO65 and 3949 FCWO65) were thawed and cultured individually for 11 weeks in 1000 ml shaker flasks containing 100 ml Excell302 medium with neomycin.

The supernatant was harvested and filtered prior to purification of the anti-RhD rpAb.

### Clonal Diversity

The clonal diversity was assayed both on the protein level as well as on the mRNA level. The supernatant sample used to analyze the antibody composition was taken after 9 weeks of cultivation, whereas the cell sample used to analyze the mRNA composition was taken at the harvest after 11 weeks of cultivation.

### Antibody Composition:

The anti-RhD rpAb expressed from the polyclonal CHO-Flp-In (019) cell line is an IgG1 isotype antibody. Anti-RhD rpAb was purified from both aliquots (3948 and 3949) using a column with immobilized Protein A. The individual antibodies interacted with immobilized Protein A at pH 7.4, whereas contaminating proteins were washed from the column. The bound antibodies were subsequently eluted from the column at low pH value (pH 2.7). The fractions containing antibodies, determined from absorbance measurements at 280 nm, were pooled and dialyzed against 5 mM sodium acetate pH 5.

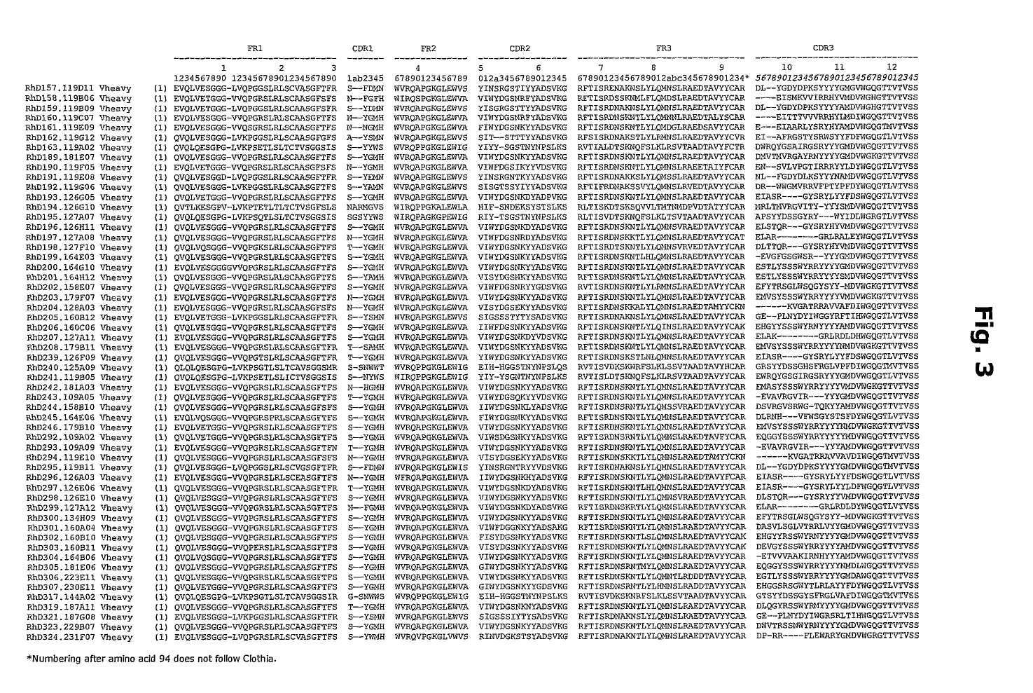

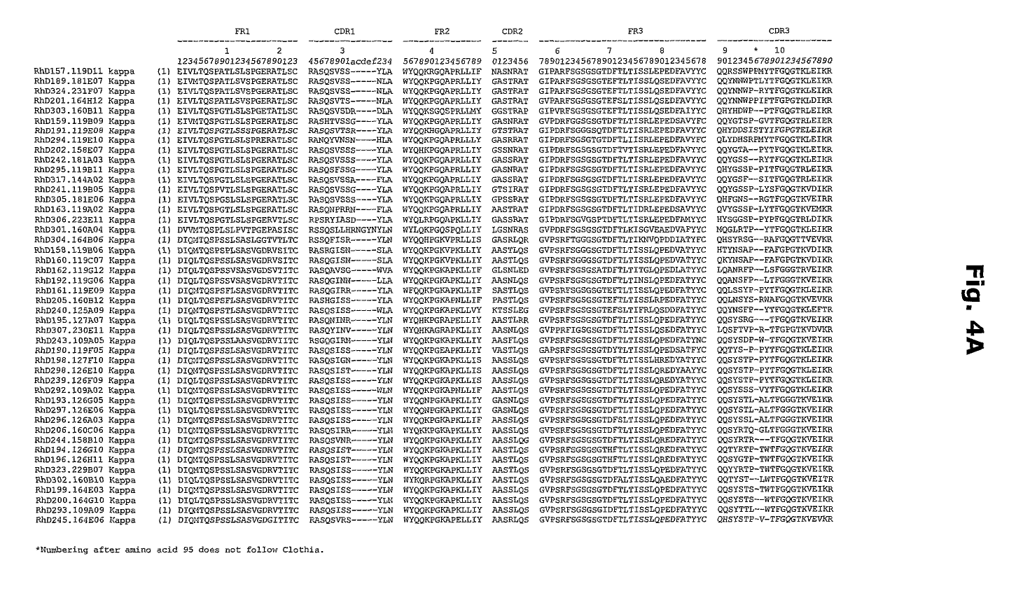

The anti-RhD rpAb compositions obtained from aliquot 3948 and 3949 (FCWO65) after 9 weeks of cultivation were analyzed using cation exchange chromatography. The Protein A purified anti-RhD rpAb was applied onto a PolyCatA column (4.6×100 mm) in 25 mM sodium acetate, 150 mM sodium chloride, pH 5.0 at a flow rate of 60 ml h⁻¹ operated at room temperature. The antibody components were subsequently eluted using a linear gradient from 150-350 mM sodium chloride in 25 mM sodium acetate, pH 5.0 at a flow rate of 60 ml h⁻¹. The antibody components were detected spectrophotometrically at 280 nm. The chromatogram (FIG. 5) was subsequently integrated and the area of the individual peaks A-J was subsequently used to quantitate antibody components (table 4). The total area of the peaks was set to 100%. The chromatograms from the two aliquots showed an identical peak distribution, as well as similar concentrations of the components in each peak. From these results it can be concluded that aliquots of the same polyclonal cell line grown under identical conditions will produce anti-RhD rpAb with a similar clonal diversity.

The individual members of the anti-RhD rpAb were allocated to one or more particular peaks (summarized in table 4). The allocation is based on chromatograms obtained for antibody products from each individual clone. No individual chromatogram was obtained for antibodies produced from RhD158.119B06, thus this clone was not assigned to any of the peaks. However it is considered likely that peak D constitute RhD158.119B06, the clone may also be represented in some of the other peaks due to heterogeneity. In particular the antibody product from clone RhD197.127A08 has a high degree of heterogeneity. Clone RhD190.119F05 should have been visible at 15.3 min. However, it was not detectable, indicating that this clone has been lost from the recombinant polyclonal manufacturing cell line. The loss of clone RhD190.119F05 corresponds to a 10% reduction of diversity which is considered acceptable with respect to diversity of the final anti-RhD rpAb composition.

TABLE 4

| | Quantity | Quantity | | |
|---|---|---|---|---|
| | 3948 | 3949 | | |
| Peak | (% area) | (% area) | Clone name | Comment |
| A | 5.1 | 5.1 | RhD157.119D11 | Clone is also present |
| | | | | in peak B |
| B | 12.0 | 10.2 | RhD157.119D11 | This peak represent at |
| | | | RhD159.119B09 | least three different |
| | | | RhD192.119G06 | clones |
| C | 5.2 | 5.3 | RhD191.119E08 | |
| D | 1.2 | 0.8 | (RhD158.119B06) | Not actually allocated to |
| | | | | this peak, but it is |
| | | | | likely to be. May also be |
| | | | | represented in other peaks. |
| E | 10.9 | 14.4 | RhD204.128A03 | |
| F | 24.3 | 23.0 | RhD197.127A08 | This clone split into |
| G | 13.6 | 12.5 | RhD197.127A08 | several peaks, due to |
| H | 3.3 | 4.0 | RhD197.127A08 | heterogeneity. |
| I | 14.0 | 13.7 | RhD161.119E09 | |
| J | 10.5 | 10.5 | RhD163.119A02 | |
| | | | RhD190.119F05 | The clone has been lost |

mRNA Composition:

The clonal diversity within the polyclonal CHO-Flp-In (019) cell line after 11 weeks of cultivation was estimated by RT-PCR-RFLP analysis. Briefly, a cell suspension corresponding to 200 cells were subjected to a freeze-thaw procedure and these lysates were used as template in a RT-PCR using One-STEP RT-PCR kit (Qiagen) with primers amplifying the light chain. The primer sequences were:

| forward primer | |
|---|---|
| 5′-CGTTCTTTTTCGCAACGGGTTTG | (SEQ ID 259) |
| reverse primer | |
| 5′-AAGACCGATGGGCCCTTGGTGGA | (SEQ ID 260) |

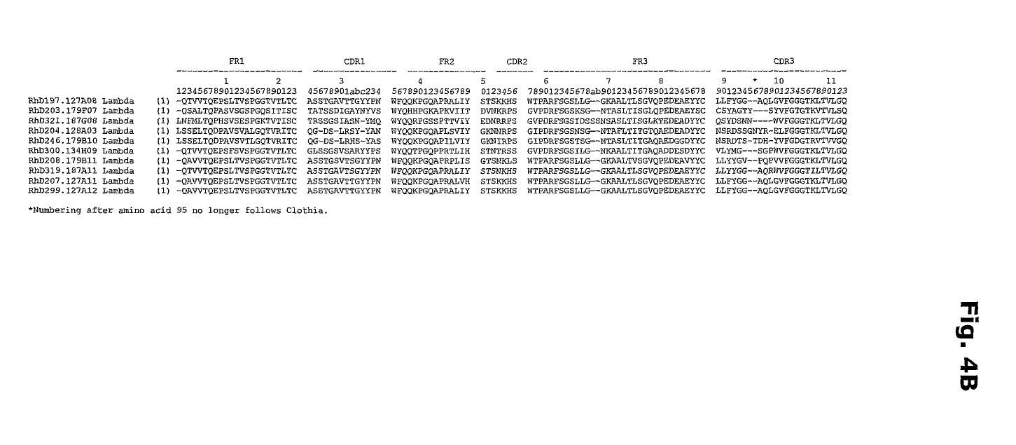

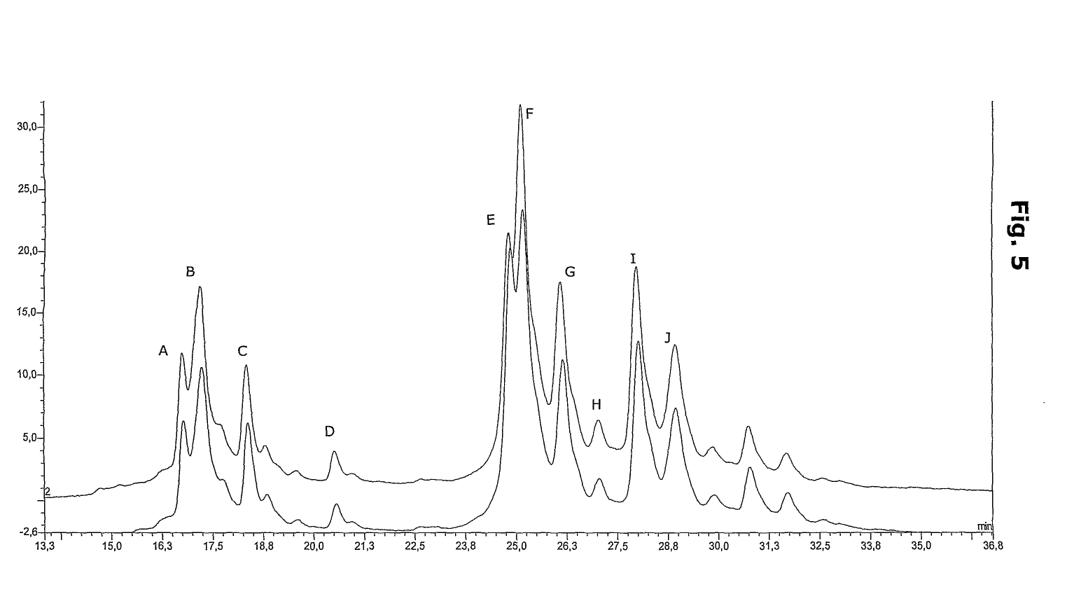

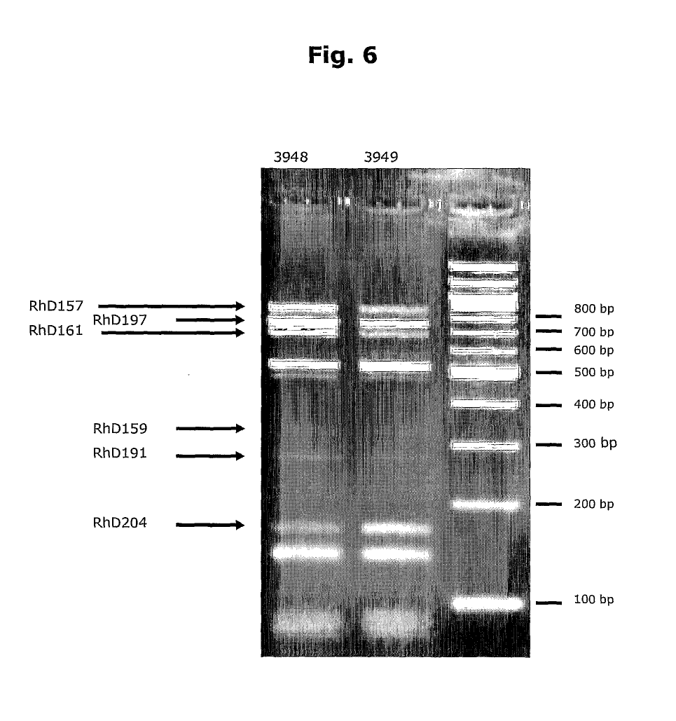

The RT-PCR products were digested with HinfI and analyzed by agarose gel electrophoresis, visualizing the restriction product with ethidium bromide staining (FIG. 6).

The expected size of the restriction fragments obtained by HinfI digestion of the RT-PCR amplified light chains are shown for each individual clone in table 5. Six unique fragment sizes on the gel, which could be assigned to specific Rhesus D antibody producing clones, are indicated in bold. Not all unique fragments could be identified on the gel, these are indicated in italic. This does, however not necessarily mean that these clones are not represented in the culture, the fragments may either not have been separated sufficiently from other fragments to be identifiable, or their concentration is to weak compared to the stronger bands. This may be more pronounced for shorter fragments, since they bind a smaller number of ethidium bromide molecules and therefore are less visible.

TABLE 5

| RhD # | 157 | 158 | 159 | 161 | 163 | 190 | 191 | 192 | 197 | 204 |
|---|---|---|---|---|---|---|---|---|---|---|
| HinfI | 825 | 671 | 505 | 696 | 505 | 502 | 475 | 671 | 743 | 521 |
| fragment | 138 | 138 | 320 | 138 | 166 | 191 | 268 | 149 | 138 | 167 |
| size | 76 | 126 | 138 | 126 | 154 | 138 | 138 | 138 | 85 | 138 |
| | | 76 | 77 | 76 | 138 | 126 | 85 | 76 | 76 | 88 |
| | | 22 | | | 76 | 76 | 76 | | | |

The two aliquots (3948 and 3949) of the same polyclonal cell line, show a similar expression pattern in the gel, although the intensity of the bands are not completely identical, this also indicates that aliquots of the same polyclonal cell line grown under identical conditions will produce anti-RhD rpAb with a similar clonal diversity.

### Summary

The present experiment succeeded in generating a library of anti-Rhesus D antibody expression vectors comprising 56 variant anti-Rhesus D-encoding nucleic acid segments (Table 3).

Plasmids containing individual members of the library were used to transfect the CHO-Flp-In (019) cell line, generating 56 individual cell lines capable of expressing a specific anti-RhD antibody.

10 of these cell lines were mixed in order to generate a anti-RhD rpAb manufacturing cell line, which after 9 weeks cultivation still maintained 90% of the initial diversity. After 11 weeks of cultivation mRNA from six different clones could be unambiguously identified and several other clones are likely to be represented in the band an approximately 500 bp.

The fact that two aliquots of the polyclonal CHO-Flp-In (019) cell lines showed similar results with respect to clonal diversity, illustrated that reproducible results can be obtained.

### Example 2

### Generation of a Polyclonal Cell Pool for Larger Scale Production

Twenty seven cell cultures were selected to constitute the polyclonal cell line (RhD157.119D11, RhD159.119B09, RhD160.119C07, RhD161.119E09, RhD162.119G12, RhD163.119A02, RhD189.181E07, RhD191.119E08, RhD192.119G06, RhD196.126H11, RhD197.127A08, RhD199.164E03, RhD201.164H12, RhD202.158E07, RhD203.179F07, RhD207.127A11, RhD240.125A09, RhD241.119B05, RhD244.158B10, RhD245.164E06, RhD293.109A09, RhD301.160A04, RhD305.181E06, RhD306.223E11, RhD307.230E11, RhD319.187A11 and RhD324.231F07).

In addition to the high degree of diversity among the individual clones, the clone selections were also based on growth and production characteristics of the individual cell cultures.

Included in the selection criteria at the cell culture level were:

I. Doubling time; had to be between 24 and 32 hours
II: Intracellular staining; had to show a homogenous cell population
III: Productivity; had to exceed 1.5 pg per cell per day

The 27 different cell cultures will be equally mixed in regard to cell number and this mix will constitute the polyclonal cell pool for a pilot plant production of anti-RhD rpAb.

### Example 3

The present example demonstrates that an anti-RhD recombinant polyclonal antibody (rpAb) with 25 individual members and the plasmaderived anti-D product WinRho®, Baxter show comparable biological activity with respect to phagocytosis, whereas an anti-RhD rpAb show less Antibody-dependent cellular cytotoxicity (ADCC).

### Preparation of Red Blood Cells—Frozen

Red blood cells (RBC) from whole blood obtained from healthy donors after informed consent at the Blood Bank, Aalborg Hospital, DK, were frozen by the high glycerol technique (38%) and stored at −80° C. The erythrocytes were thawed in 12% NaCl (Merck) and citrate-manitol (LAB20910.0500, Bie & Berntsen) was added after 3 min. The cells were washed 3 times in PBS (Invitrogen, CA, US) and stored at 4° C. as a 3% solution in ID-Cellstab (DiaMed, Switzerland).

### Preparation of PBMC

Buffy coats containing blood from healthy donors were obtained from the Blood Bank at the National Hospital, Copenhagen, Denmark and peripheral blood mononuclear cells (PBMC) were purified on Lymphoprep (Axis-Shield, Norway). Pooled PBMC could be frozen in 10% DMSO (Sigma) and stored at −80° C.

### Combined ADCC and Phagocytosis Assay

This assay was adapted from Berkman et al. 2002. Autoimmunity 35, 415-419. Briefly, RhD positive (RhD+) or RhD negative (RhD−) red blood cells (RBC) were labeled with radioactive Chromium. For Cr51 labeling, 1×10⁸ RhD+ and RhD− RBC, respectively, were centrifuged (700×g for 2 min) and 100 μl RPMI ((Invitrogen, CA, US)) and 200 μl sodium chromate (0.2 μCi) (GE Healthcare, UK) were added to each tube before incubation for 1.5 hours at 37° C. The suspension was centrifuged (2 min 700×g) and supernatant removed. Then the RBCs were washed twice in 15 ml PBS and resuspended in PBS with 0.1% BSA (Sigma). Cells were adjusted to 2×10⁶ cells/ml and 50 μl/well were added to 96-well cell culture plates (Nunc). Fifty μl of two-fold dilutions in PBS with 0.1% BSA of Anti-RhD rpAb produced at Biovitrum (Sym001 rWS (research working standard) further described in WO 2006/007850 A1 Example 5) and the plasmaderived anti-D product WinRho®, Baxter, was then added to each well, except control wells. The plates incubate 40 min at 37° C. in the heating cupboard. Hereafter the cells are carefully washed (2 min 700×g) three times in 200 μl/well PBS and resuspended in 100 μl/well complete RPMI.

The PBMC were adjusted to 2×10⁷ cells/ml and 100 μl were added to each well. Control wells were supplied with complete RPMI and used for either spontaneous lysis/retention or maximum lysis. The plate was incubated at 37° C. overnight in a humified incubator. One hundred μl 1% Triton-X-100 (Merck, Germany) was added to the maximum lysis control wells. The plates were centrifuged (700×g for 2 min) and 50 μl of the supernatant was transferred to ADCC Lumaplates (Perkin Elmer, Belgium).

Following transfer of the supernatants, the cell culture plates were centrifuged (700×g for 2 min) and 50 μl supernatant from the maximum lysis wells were transferred to another LumaPlate (phagocytosis LumaPlate). In the cell culture plate, the supernatant was removed from the remaining wells and lysis buffer (140 mM NH4Cl, 17 mM Tris-HCl) was added, followed by 10 min incubation at 37° C. NH4Cl lyses the RBC, but leaves the PBMC fraction and thereby the phagocytozed RBC intact. After RBC lysis, the plates were centrifuged (700×g for 2 min), pellets were washed twice in PBS, and resuspended in 100 μl PBS. One hundred μl 1% Triton-X-100 was added to the wells to lyse the phagocytic PBMC, and 50 μl of the lysate was transferred to the phagocytosis LumaPlates. The LumaPlates were dried overnight at 37° C. and counted in a TopCount NXT (Packard, Conn., USA). All data were imported into Excel and analyzed as described by Berkman et al. 2002. Autoimmunity 35, 415-419. Briefly, the computations were performed as follows:

ADCC: Immune lysis (%)=(mean test Cr51 released−mean spontaneous Cr51 released)/(total Cr51 in target erythrocytes-machine background)×100

Phagocytosis: Immune phagocytosis (%)=(mean test Cr51 retention−mean spontaneous Cr51 retention)/(total Cr51 in target erythrocytes-machine background)×100

All data were normalized to the combined maximum plateau values

The functional activity of anti-RhD rpAb produced at Biovitrum and WinRho®, Baxter showed nearly identical functional activity in regards to phagocytosis but the anti-RhD rpAb showed less activity in regards to ADCC.

### Example 4

A functional assay which better represents mechanism of action of anti-D in ITP has been developed. In this ITP model an anti-RhD rpAb produced at Biovitrum and WinRho®, Baxter showed nearly identical functional activity in regards to dose dependent inhibition of platelet phagocytosis.

Breifly, in this study platelets are labeled with CM green and opsonized with antibodies and incubated with effector cells: THP-1a monocytic cell line.

### Preparation of Platelets

Buffy coats containing blood from healthy donors were obtained from the Blood Bank at the National Hospital, Copenhagen, Denmark and platelets were purified by centrifugation (210 g, 15 min, RT no brakes). Platelet rich plasma was collected, CPDA (citrate-phosphate-dextran solution with adenine, Sigma) was added and platelets were spun (580 g, 15 min, RT, no brakes).

Platelets were resupended in 10% CPDA in PBS with 0.02 mg/ml Prostaglandin E1, Sigma and 20 μM CM green, Invitrogen. Platelets were incubated 20 min at 37° C., washed once (580 g, 15 min, RT, no brakes), left ON at RT, washed once and resuspended to 5×10⁸ platelets/ml in 10% CPDA in PBS. 100 μl w6/32 (mlgG anti HLA1), Sigma (0.2 mg/ml) was added to every 1 ml platelet solution. Platelets were incubated 30 min at RT, washed once and resuspend to 2×10⁸ platelets/ml in 10% CPDA in PBS.

### Preparation of Red Blood Cells—Frozen

Red blood cells (RBC) from whole blood obtained from healthy donors after informed consent at the Blood Bank, Aalborg Hospital, DK, were frozen by the high glycerol technique (38%) and stored at −80° C. The erythrocytes were thawed in 12% NaCl (Merck) and citrate-manitol (LAB20910.0500, Bie & Berntsen) was added after 3 min. The cells were washed 3 times in PBS (Invitrogen, CA, US) and stored at 4° C. as a 3% solution in ID-Cellstab (DiaMed, Switzerland).

RBC were washed once in PBS (700 g, 5 min) and adjusted to 4×10⁸ RBC/ml in PBS and 50 μl/well were added to 96-well FACS plates (BD). Fifty μl of two-fold dilutions in PBS with 0.1% BSA of Anti-RhD rpAb produced at Biovitrum (Sym001 rWS (research working standard) further described in WO 2006/007850 A1 Example 5) and the plasmaderived anti-D product WinRho®, Baxter, was then added to each well, except control wells. The plate incubated 45 min at RT on a plate shaker. Hereafter the cells are carefully washed (2 min 700×g) twice in 200 μl/well PBS and resuspended in 16% Iscove's DMEM in PBS.

### Preparation of THP-1 Cells

THP-1 cells were cultured in a humified incubator (5% CO₂— 37° C.) in complete RPMI (+glutamax, 10% fetal calf serum, 1% Penicillin-Streptomycin) (Invitrogen, CA, US). THP-1 cells were spun and washed once with PBS (22° C. 300×g 7 min) and resuspended in PBS to 1×10⁷ cells/ml. The cells are stimulated with 0.1 μg PMA(Sigma)/10⁷ cells (10 μl of a 100×PBS diluted stock of 1 mg/ml) 15 min at RT. The cells are washed once in PBS and adjusted to 1×10⁷ cells/ml in 16% Iscove's DMEM in PBS.

### Phagocytosis of Platelets

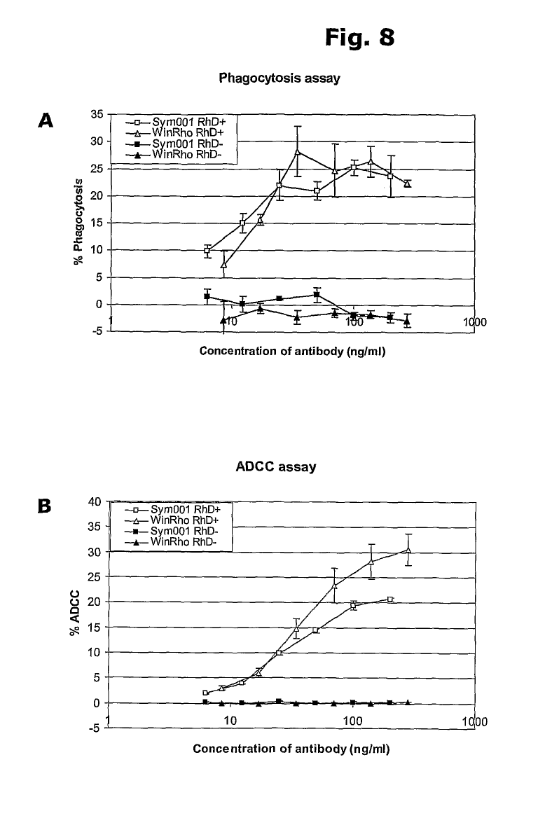

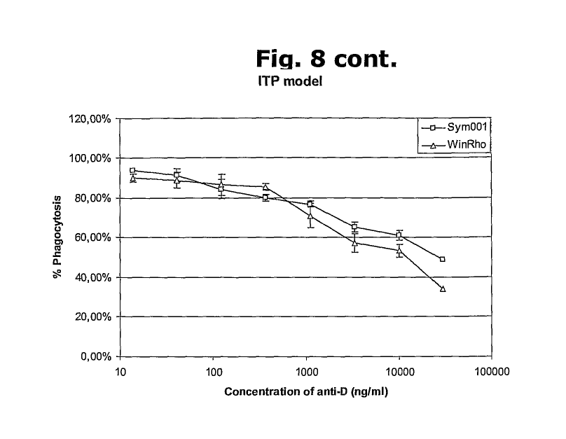

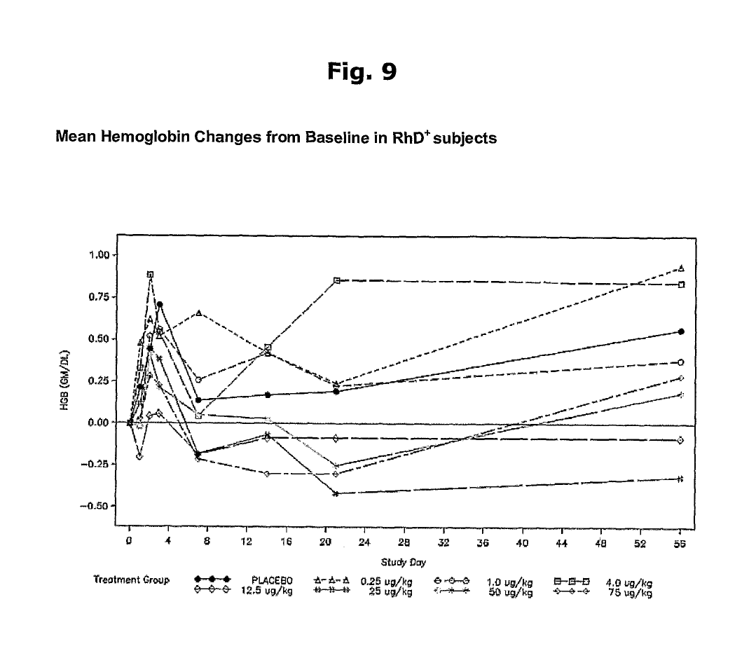

Platelets (2×10⁸/ml 50 μl/well for 1:20 E:T ratio), RBC (4×10⁸/ml 50 μl/well for 1:40 E:T ratio) and THP-cells (1×10⁷/ml 50 μl/well) were mixed and incubated 2 h in a humified incubator (5% CO₂— 37° C.). 100 μl Trypan Blue, Fluka prediluted 1:1 in PBS was added to block the nonspecific binding on the outside of THP-cells. Washed once in 200 μl/well PBS (210 g +4° C., 3 min), 200 μl/well Lysing solution, BD was added and incubated 15 min at 4° C. Washed once in 200 μl/well PBS and resuspended in 200 μl/well PBS. Cells were acquired live gate thru SSC and FSC on HTS on FACS Calibur and the Median fluorescence intensity of FI-1 was analyzed (FIG. 8A-C).

### Example 5

The present example demonstrates the generation of pWCP containing anti-RhD rpAb with 25 individual members and provides confirmation of a minimal batch-to-batch variation of rpAb products purified from different vials from the pWCP.

Generation of the pWCP

To generate a pWCP containing anti-RhD rpAb with 25 individual members, one vial of each of 25 banked monoclonal anti-RhD antibody production cell lines (RhD157, 159, 160, 162, 189, 191, 192, 196, 197, 199, 201, 202, 203, 207, 240, 241, 245, 293, 301, 305, 306, 317, 319, 321, 324) were thawed in ExCell 302 medium containing 4 mM glutamine and expanded for 3 weeks in the same medium supplemented with 500 g/ml G418 and anti-clumping agent diluted 1:250. Equal numbers of cells (2×10⁶) from each culture were then carefully mixed together, and frozen in liquid nitrogen (5×10⁷ cells/vial) using standard freezing procedures.

### Cultivation in Bioreactors

Vials from the pWCP were thawed in T75 flasks (Nunc, Roskilde, Denmark) and expanded in spinner flasks (Techne, Cambridge, UK). 5 L bioreactors (Applikon, Schiedam, Netherlands) were inoculated with 0.6×10⁶ cells/ml in 1.5 L. During the reactor runs, cells were fed on a daily basis with ExCell 302 medium supplemented with concentrated feed solution, glutamine and glucose to a final volume of 4.5 L. The bioreactor runs were terminated after 16-17 days. The three batches are termed Sym04:21, Sym04:23 and Sym04:24. The batches were cultured at different points in time.

### Analysis of Batch-to-Batch Variation

The recombinant polyclonal antibody samples were purified by affinity chromatography using HiTrap™ rProtein A columns (GE Healthcare, UK).

The purified recombinant polyclonal antibody samples were analyzed using cation-exchange chromatography employing a PolyCAT A column (4.6×100 mm, from PolyLC Inc., MA, US) in 25 mM sodium acetate, 150 mM sodium chloride, pH 5.0 at a flow rate of 60 ml/h (room temperature). The antibody peaks were subsequently eluted using a linear gradient from 150 mM to 350 or 500 mM NaCl in 25 mM sodium acetate, pH 5.0 at a flow rate of 60 ml/h. The antibody peaks were detected spectrophotometrically at 280 nm. The chromatograms were integrated and the area of individual peaks used for quantification. As already mentioned some of the individual antibodies displayed charge heterogeneity and two antibodies may contribute to the same peak in the IEX chromatogram.

Table 6 show the relative content in percent of the total antibody components (AC). In the present example the relative area has been calculated for 35 AC, whereas Example 4 only calculated the relative area for 25 AC. This difference is strictly due to a different assignment of the peaks in the chromatogram and not to actual differences in the profile as such.

TABLE 6

| | Average | Standard |
|---|---|---|
| Peak | Rel. Area % | deviation |
| AC 1 | 1.71 | 0.35 |
| AC 2 | 2.36 | 0.13 |
| AC 3 | 4.40 | 0.78 |
| AC 4 | 3.58 | 0.78 |
| AC 5 | 5.83 | 0.60 |
| AC 6 | 2.11 | 0.25 |
| AC 7 | 4.16 | 0.33 |
| AC 8 | 4.21 | 0.59 |
| AC 9 | 3.41 | 0.97 |
| AC 10 | 14.22 | 2.91 |
| AC 11 | 4.24 | 0.79 |
| AC 12 | 2.98 | 0.47 |
| AC 13 | 2.31 | 0.16 |
| AC 14 | 2.44 | 0.26 |
| AC 15 | 9.17 | 0.52 |
| AC 16 | 5.08 | 0.43 |
| AC 17 | 1.98 | 0.26 |
| AC 18 | 3.04 | 0.26 |
| AC 19 | 1.79 | 0.16 |
| AC 20 | 1.39 | 0.07 |
| AC 21 | 1.32 | 0.15 |
| AC 22 | 2.60 | 0.23 |
| AC 23 | 1.59 | 0.25 |
| AC 24 | 0.62 | 0.12 |
| AC 25 | 1.12 | 0.06 |
| AC 26 | 1.31 | 0.04 |
| AC 27 | 0.58 | 0.12 |
| AC 28 | 1.30 | 0.25 |
| AC 29 | 1.05 | 0.39 |
| AC 30 | 0.66 | 0.24 |
| AC 31 | 0.70 | 0.44 |
| AC 32 | 1.64 | 0.10 |
| AC 33 | 2.30 | 0.16 |
| AC 34 | 1.77 | 0.24 |
| AC 35 | 1.03 | 0.44 |

Table 6 shows that the reproducibility between the harvested antibody products from the three batches was high. The variation in the size of individual antibody peaks was within 20% for most antibody components, whereas the variation for some of the smallest peaks was slightly larger.

### Example 6

The present example demonstrates that different batches of an anti-RhD rpAb with 25 individual members (same composition as in Example 4) bind to RhD-positive erythrocytes with similar potency and show comparable biological activity with respect to the relevant effector mechanisms: Antibody-dependent cellular cytotoxicity (ADCC) and phagocytosis.

### Preparation of Red Blood Cells

Red blood cells (RBC) were prepared from whole blood obtained from healthy donors after informed consent at the Blood Bank, Aalborg Hospital, DK, by washing the blood three times in PBS (Gibco, Invitrogen, United Kingdom) containing 1% bovine serum albumin (BSA, Sigma-Aldrich, Germany). The erythrocytes were resuspended and stored at 4° C. as a 10% solution in ID-Cellstab (DiaMed, Switzerland).

### Preparation of PBMC

Buffy coats containing blood from healthy donors were obtained from the Blood Bank at the National Hospital, Copenhagen, Denmark and peripheral blood mononuclear cells (PBMC) were purified on Lymphoprep (Axis-Shield, Norway).

### Potency Assay

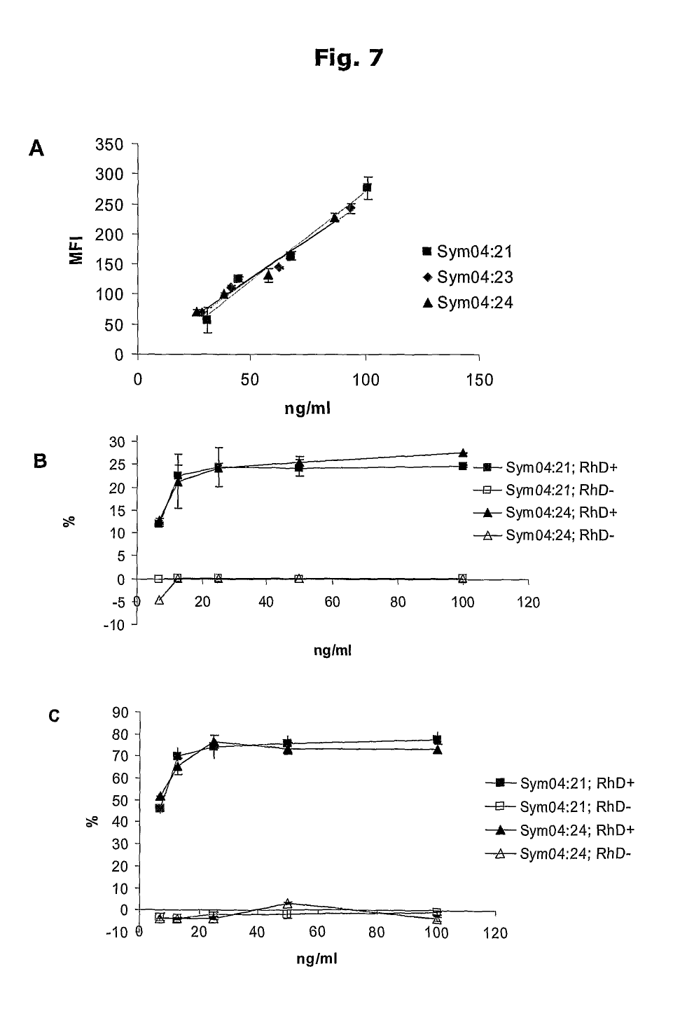

The potency assay was adopted from the European Pharmacopoeia 4 (section 2.7.13 method C). The binding capacity of an anti-RhD rpAb with 25 individual members was measured using RhD-positive erythrocytes at 5×10⁴ cells/μl in PBS, 1% BSA. Anti-RhD rpAb batches, Sym04:21, Sym04:23, and Sym04:24, were obtained from individual 5 L fed batch bioreactor runs. Dilutions (1½-fold) of the Anti-RhD rpAb batches were made in PBS, 1% BSA in triplicate in 96 well plates (Becton Dickinson Labware, NJ, USA). Fifty μl of the anti-RhD rpAb dilutions were mixed with 50 μl of erythrocytes and incubated at 37° C. for 40 min. The cells were washed twice (300×g, 2 min) in PBS, 1% BSA. Eighty μl of phycoerythrin-conjugated goat anti-human IgG, (Beckman Coulter, CA, USA) diluted 1:20 in PBS, 1% BSA was added to each sample and left at 4° C. for 30 min. The samples were washed in PBS, 1% BSA and in FacsFlow (Becton Dickinson, Belgium) (300×g, 2 min), and resuspended in 200 μl FACSFlow. The samples were run on a FACSCalibur (Becton Dickinson, CA, USA) and data analysis performed using CellQuest Pro and Excel. The three individual Anti-RhD rpAb batches displayed essentially identical binding potency to RhD-positive erythrocytes (FIG. 7A)

### Combined ADCC and Phagocytosis Assay

This assay was adapted from Berkman et al. 2002. Autoimmunity 35, 415-419. Briefly, RhD positive (RhD+) and RhD negative (RhD−) red blood cells (RBC) were labeled with radioactive Chromium. For Cr⁵¹ labeling, 1×10⁸ RhD+ and RhD− RBC, respectively, were centrifuged (600×g for 10 min) and 100 μl Dulbeccos' modified eagles medium (DMEM) and 200 μl sodium chromate (0.2 μCi) (GE Healthcare, UK) were added to each tube before incubation for 1.5 hours at 37° C. The suspension was washed twice in 50 ml PBS and resuspended in 1 ml complete DMEM (containing 2 mM glutamine, 1% Penicillin-Streptomycin and 10% fetal calf serum) (Invitrogen, CA, US). Cells were adjusted to 4×10⁶ cells/ml and 50 μl/well were added to 96-well cell culture plates (Nunc). Fifty μl of two-fold dilutions of Anti-RhD rpAb from batch Sym04:21 or Sym04:24, was then added to each well, except control wells. Control wells were supplied with complete DMEM and used for either spontaneous lysis/retention or maximum lysis.

The PBMC were adjusted to 2×10⁷ cells/ml, and 100 μl were added to each well and incubated at 37° C. overnight. One hundred μl 1% Triton-X-100 (Merck, Germany) was added to the maximum lysis control wells. The plates were centrifuged (600×g for 2 min) and 50 μl of the supernatant was transferred to ADCC Lumaplates (Perkin Elmer, Belgium).

Following transfer of the supernatants, the cell culture plates were centrifuged (300×g for 2 min) and 50 μl supernatant from the maximum lysis wells were transferred to another LumaPlate (phagocytosis LumaPlate). In the cell culture plate, the supernatant was removed from the remaining wells and lysis buffer (140 mM NH₄Cl, 17 mM Tris-HCl) was added, followed by 5 min incubation at 37° C. NH₄Cl lyses the RBC, but leaves the PBMC fraction and thereby the phagocytozed RBC intact. After RBC lysis, the plates were centrifuged (4° C., 2 min, 300 g), pellets were washed twice in PBS, and resuspended in 100 μl PBS. One hundred μl 1% Triton-X-100 was added to the wells to lyse the phagocytic PBMC, and 50 μl of the lysate was transferred to the phagocytosis LumaPlates. The Lumaplates were dried overnight at 40° C. and counted in a TopCount NXT (Packard, Conn., USA). All data were imported into Excell and analyzed as described by Berkman et al. 2002. Autoimmunity 35, 415-419. Briefly, the computations were performed as follows:

ADCC: Immune lysis (%)=(mean test Cr⁵¹ released−mean spontaneous Cr⁵¹ released)/(total Cr⁵¹ in target erythrocytes-machine background)×100

Phagocytosis: Immune phagocytosis (%)=(mean test Cr⁵¹ retention−mean spontaneous Cr⁵¹ retention)/(total Cr⁵¹ in target erythrocytes-machine background)×100

All data were normalized to the combined maximum plateau values

The functional activity of anti-RhD rpAb from the two consecutive reactor runs showed nearly identical functional activity in both in vitro assays (FIGS. 7B and 7C) reflecting the high consistency between the batches.

### Example 7

Title of Study: A placebo-controlled, double-blind, randomized, sequential dose escalation safety, pharmacokinetic and pharmacodynamic study of a single intravenous Sym001 administration in RhD positive and RhD negative healthy volunteers

Primary Objective: To assess the safety of Sym001 following a single intravenous (IV) infusion in RhD⁻ and RhD⁺ healthy volunteers.

Methodology: Seventy-seven healthy subjects were enrolled in this double-blind, sequential dose-escalation study of the PK, PD, and safety of a single dose of Sym001 administered IV. Screening was performed between Day-28 and Day-2 for each cohort. On Day 1, subjects received Sym001 single doses of 0.25, 1.0, 4.0, 12.5, 25, 50, and 75 μg/kg. or placebo given IV over a period of 30 minutes according to a randomization schedule prepared prior to the start of the study.

Increasing dose levels of Sym001 were studied in 7 dosing cohorts, as described in table 7.

TABLE 7

| | | Number of Subjects | | | |
|---|---|---|---|---|---|
| | | RhD⁺ | RhD⁺ | RhD⁻ | RhD⁻ |
| | Dose | Sym001 | Placebo | Sym001 | Placebo |
| Cohort | (μg/kg) | (A) | (A) | (B) | (B) |
| 1 | 0.25 | 5 | 2 | 0 | 0 |
| 2 | 1.0 | 5 | 2 | 0 | 0 |
| 3A &3B | 4.0 | 7 | 2 | 7 | 2 |
| 4A &4B | 12.5 | 7 | 2 | 7 | 2 |
| 5 | 25 | 7 | 2 | 0 | 0 |
| 6 | 50 | 7 | 2 | 0 | 0 |
| 7 | 75 | 7 | 2 | 0 | 0 |

The Safety Monitoring Committee (SMC) reviewed all available safety and PD data 7 days after each cohort had been dosed and decided whether to increase the dose administered to the next cohort in sequence as planned. Escalation was allowed to proceed if there was:
No decrease in Hgb of >2.0 g/dL in any one subject per cohort
No decrease in Hgb of >1.0 g/dL in 3 or more subjects per cohort

No pattern of treatment emergent adverse events (TEAEs) rated moderate in severity and considered to be probably or possibly treatment-related where the prevalence or nature of symptoms raised potential safety concerns.

### RESULTS

### Safety Results:

There were no deaths, no serious AEs, no AEs of severe intensity, and no AE that resulted in discontinuation from the study in either the RhD or RhD⁻ population.

### Changes in Hemoglobin at 7 Days Post-Dose.

None of the changes in hemoglobin from baseline level were considered clinically significant in either RhD population during this trial. In individual subjects, there was no hemoglobin decrease of >2 g/dL. Decreases in hemoglobin of >1 g/dL occurred in 10 subjects (9 RhD⁺ and one RhD⁻) and did not appear to be associated with dose of Sym001, or with clinically significant changes in other biomarkers of hemolysis. None of the individual hemoglobin decreases of >1 g/dL resulted in a value outside of the normal range for hemoglobin in healthy male adults.

Discussion: In this trial, no substantial drop in Hb was observed in RhD⁺ subjects at doses up to 75 μg/kg. This is not in line with data on Hb fall observed with plasma-derived anti-D products. In a trial in RhD positive volunteers who received a single dose of WinRho®, the Hb fall (at 28 days) was 1.1 and 2.1 g/dL with doses of 50 and 75 μg/kg, respectively. Clinical trials in ITP patients have shown significant Hb fall following treatment with plasma-derived anti-D products. In 4 clinical trials of patients treated with the recommended initial intravenous dose of 50 μg/kg of WinRho, the mean maximum decrease in hemoglobin was 1.70 g/dL (range +0.40 to −6.1 g/dL). In a trial with 98 ITP patients treated with a single dose Rhophylac®, The greatest decrease in Hb occurred at days 6 and 8 post dose was and corresponded to (0.8 g/dL) at Day 6 and Day 8 following administration of Rhophylac®.

Given the similar potency of Sym001 and plasma-derived anti-D products in terms of RBC binding and phagocytosis in vitro, a similar effect of Sym001 and plasma-derived anti-D products on hemoglobin in vivo could be expected. The results of the first in human trial (Sym001-01) indicate that the hemoglobin fall in RhD positive subjects, following doses up to 75 μg/kg, may be less important than that observed with therapeutic doses of plasma-derived anti-D products. It might be suggested that, at therapeutic doses in ITP patients, Sym001 may cause less fall in hemoglobin than plasma-derived anti-D products, which could result in a better risk-benefit profile of Sym001.

### REFERENCES

Stasi R, Provan D. Management of immune thrombocytopenic purpura in adults. Mayo Clin Proc 2004 April; 79(4):504-22.
Cines D B, Blanchette V S. Immune thrombocytopenic purpura. N Engl J Med 2002 Mar. 28; 346(13):995-1008.
Cines D B, McMillan R. Management of adult idiopathic thrombocytopenic purpura. Annu Rev Med 2005; 56:425-42.
George J N, Woolf S H, Raskob G E, Wasser J S, Aledort L M, Ballem P J et al. Idiopathic thrombocytopenic purpura: a practice guideline developed by explicit methods for the American Society of Hematology. Blood 1996 Jul. 1; 88(1):3-40.
Blanchette V, Imbach P, Andrew M, Adams M, McMillan J, Wang E et al. Randomised trial of intravenous immunoglobulin G, intravenous anti-D, and oral prednisone in childhood acute immune thrombocytopenic purpura. Lancet 1994 Sep. 10; 344(8924):703-7.
Bussel J B, Graziano J N, Kimberly R P, Pahwa S, Aledort L M. Intravenous anti-D treatment of immune thrombocytopenic purpura: analysis of efficacy, toxicity, and mechanism of effect. Blood 1991 May 1; 77(9):1884-93.
Cooper N, Woloski B M, Fodero E M, Novoa M, Leber M, Beer J H et al. Does treatment with intermittent infusions of intravenous anti-D allow a proportion of adults with recently diagnosed immune thrombocytopenic purpura to avoid splenectomy? Blood 2002 Mar. 15; 99(6):1922-7.
Newman G C, Novoa M V, Fodero E M, Lesser M L, Woloski B M, Bussel J B. A dose of 75 microg/kg/d of i.v. anti-D increases the platelet count more rapidly and for a longer period of time than 50 microg/kg/d in adults with immune thrombocytopenic purpura. Br J Haematol 2001 March; 112(4):1076-8.
Sandler S G. Intravenous Rh immune globulin for treating immune thrombocytopenic purpura. Curr Opin Hematol 2001 November; 8(6):417-20.
Scaradavou A, Woo B, Woloski B M, Cunningham-Rundles S, Ettinger L J, Aledort L M et al. Intravenous anti-D treatment of immune thrombocytopenic purpura: experience in 272 patients. Blood 1997 Apr. 15; 89(8):2689-700.
Bussel J B, Kaufmann C P, Ware R E, Woloski B M. Do the acute platelet responses of patients with immune thrombocytopenic purpura (ITP) to IV anti-D and to IV gammaglobulin predict response to subsequent splenectomy? Am J Hematol 2001 May; 67(1):27-33.
International standard for minimum potency of the antii-D blood grouping reagents. Report of the International Collaboration Study to evaluate a proposed WHO international standard for minimum potency of anti-D blood grouping reagents. http://www.who int/bloodproducts/cs/042000rev1 pdf 2005;
FDA. Drug Warning. Dec. 5, 2005. Ref Type: Internet Communication
Gaines A R. Disseminated intravascular coagulation associated with acute hemoglobinemia or hemoglobinuria following Rh(0)(D) immune globulin intravenous administration for immune thrombocytopenic purpura. Blood 2005 Sep. 1; 106(5):1532-7.
Schwartz J, Spitalnik S, Grima K M. Severe hemolysis following administration of Rh(o)(D) immune globulin in an ITP patient associated with anti-C. Blood 2006 Mar. 15; 107(6):2585.
Avent N D, Liu W, Scott M L, Jones J W, Voak D. Site directed mutagenesis of the human Rh D antigen: molecular basis of D epitopes. Vox Sang 2000; 78 Suppl 2:83-9.
Scott M L, Voak D, Liu W, Jones J W, Avent N D. Epitopes on Rh proteins. Vox Sang 2000; 78 Suppl 2:117-20.
Kumpel B M, Goodrick M J, Pamphilon D H, Fraser I D, Poole G D, Morse C et al. Human Rh D monoclonal antibodies (BRAD-3 and BRAD-5) cause accelerated clearance of RhD+red blood cells and suppression of Rh D immunization in Rh D-volunteers. Blood 1995 Sep. 1; 86(5):1701-9.
Kumpel B M, De H M, Koene H R, Van De Winkel J G, Goodrick M J. Clearance of red cells by monoclonal IgG3 anti-D in vivo is affected by the VF polymorphism of Fcgamma RIIIa (CD16). Clin Exp Immunol 2003 April; 132(1):81-6.
Miescher S, Spycher M O, Amstutz H, De H M, Kleijer M, Kalus U J et al. A single recombinant anti-RhD IgG prevents RhD immunization: association of RhD-positive red blood cell clearance rate with polymorphisms in the FcgammaRIIA and FcgammaIIIA genes. Blood 2004 Jun. 1; 103(11):4028-35.
Andersen P S, Haahr-Hansen M, Coljee V W, Hinnerfeldt F R, Varming K, Bregenholt S et al. Extensive restrictions in the VH sequence usage of the human antibody response against the Rhesus D antigen. Mol Immunol 2007 January; 44(4):412-22.
Lazarus A H, Crow A R. Mechanism of action of IVIG and anti-D in ITP. Transfus Apher Sci 2003 June; 28(3):249-55.
Salama A, Mueller-Eckhardt C, Kiefel V. Effect of intravenous immunoglobulin in immune thrombocytopenia. Lancet 1983 Jul. 23; 2(8343):193-5.
Salama A, Kiefel V, Amberg R, Mueller-Eckhardt C. Treatment of autoimmune thrombocytopenic purpura with rhesus antibodies (anti-Rh0(D)]. Blut 1984 July; 49(1):29-35.
Jefferis R. Glycosylation of Recombinant IgG Antibodies and its Relevance for Therapeutic Applications. Cell Engineering. Kluwer Academic Publishers; 2002. p. 93-107.

1. A recombinant polyclonal anti-RhesusD antibody product for use in the treatment or prophylaxis of thrombocytopenia, wherein said antibody product is prepared for administration in a dose of 10-500 microgram specific antibody/kg patient body mass, said recombinant polyclonal anti-RhesusD antibody product comprising a defined subset of individual antibodies, exhibiting binding to at least one epitope on the Rhesus D antigen.

2. The antibody product of claim 1, wherein said at least one epitope is selected from the group consisting of epD1, epD2, epD3, epD4, epD5, epD6/7, epD8, epD9, and combinations thereof.

3. The antibody product of claim 1, wherein at least one of the individual antibodies specifically binds to epD3, epD4 and epD9 (RhD category VI antigen) and further individual antibodies alone or in combination bind to the remaining Rhesus D antigen epitopes epD1, epD2, epD5, epD6/7 and epD8.

4. The antibody product of claim 1, wherein said individual antibodies do not bind or only weakly bind to Rhesus C, c, E, and e antigens.

5. The antibody product of claim 1, wherein said antibody is administered in a dose of:
a) from 10-25 microgram specific antibody/kg patient body mass;
b) from 25-50 microgram specific antibody/kg patient body mass;
c) from 50-75 microgram specific antibody/kg patient body mass;
d) from 75-100 microgram specific antibody/kg patient body mass;
e) from 100-125 microgram specific antibody/kg patient body mass;
f) from 125-150 microgram specific antibody/kg patient body mass;
g) from 150-175 microgram specific antibody/kg patient body mass;
h) from 175-200 microgram specific antibody/kg patient body mass;
i) from 200-225 microgram specific antibody/kg patient body mass;
i) from 225-250 microgram specific antibody/kg patient body mass;
k) from 250-275 microgram specific antibody/kg patient body mass;
l) from 275-300 microgram specific antibody/kg patient body mass;
m) from 300-325 microgram specific antibody/kg patient body mass;
n) from 325-350 microgram specific antibody/kg patient body mass;
o) from 350-375 microgram specific antibody/kg patient body mass;
p) from 375-400 microgram specific antibody/kg patient body mass;
q) from 400-425 microgram specific antibody/kg patient body mass;
r) from 425-450 microgram specific antibody/kg patient body mass;
s) from 450-475 microgram specific antibody/kg patient body mass; or
t) from 475-500 microgram specific antibody/kg patient body mass.

6. The antibody product of any of claim 1 to 5, wherein said thrombocytopenia is treated in a subject with anaemia.

7. The antibody product of claim 6, wherein the subject has a haemoglobin level, which is more than 2× the standard deviation below the average for the gender and age to which the subject belongs.

8. The antibody product of claim 6, wherein the subject has a haemoglobin level lower than 2.0 g/dL below the lower limit of the laboratory normal range for gender and age.

9. The antibody product of claim 6, wherein the subject has a haemoglobin level of less than 10 g/dL.

10. The antibody product of claim 6, wherein the subject is an adult female with a haemoglobin level of less than 12-16 g/dL such as less than 12 g/dL.

11. The antibody product of claim 6, wherein the subject is an adult male with a haemoglobin level of less than 13-18 g/dL such as less than 14 g/dL.

12. The antibody product of claim 6, wherein the subject is a pregnant woman with a haemoglobin level of less than 11-12 g/dL.

13. The antibody product of claim 6, wherein the subject is a newborn with a haemoglobin level of less than 17-19 g/dL.

14. The antibody product of claim 6, wherein the subject is a child with a haemoglobin level of less than 14-17 g/dL.

15. The antibody product of claim 6, wherein the subject has a haemoglobin level of less than 2× lower than the normal level for the age and gender of the subject and wherein the dose of recombinant polyclonal anti-RhesusD antibody product is not affected by the haemoglobin level of the subject.

16. The antibody product of claim 1, wherein administration of said antibody leads to a fall in the haemoglobin level in 90% of the treated subjects of no more than 30%.

17. The antibody product of claim 1, wherein said dose does not substantially lead to extravascular haemolysis.

18. The antibody product of claim 1, wherein administration of said antibody does not lead to transfusion requiring anemia.

19. The antibody product of claim 1, wherein the subject is RhesusD positive.

20. The antibody product of claim 1, wherein the subject is RhesusD negative.

21. The antibody product of claim 1, wherein the subject is non-splenectomised.

22. The antibody product of claim 1, wherein the subject is splenectomised.

23. The antibody product of claim 1, wherein the thrombocytopenia involves an immune component.

24. The antibody product of claim 1, wherein the thrombocytopenia does not involve an immune component.

25. The antibody product of claim 23, wherein the thrombocytopenia can be selected from the group consisting of ITP, Antiphospholipid antibody syndrome, Acquired selective amegacariyocyte aplasia, Immune thrombocytopenia caused by infectious agents, especially, but not exclusively Helicobacter pylori, HIV and/or Hepatitis C, Neonatal auto- or alloimmune thrombocytopenia (due to maternal transplacentally transferred antibody) and Drug induced thrombocytopenia with drug dependent antibodies.

26. The antibody product of claim 24, wherein the thrombocytopenia can be selected from the group consisting of Congenital thrombocytopenias, Thrombotic Thrombocytopenic Purpura, thrombocytopenia caused by Hemolytic-uremic syndrome, thrombocytopenia caused by Chemotherapy, thrombocytopenia caused by Radiation, thrombocytopenia caused by Nutritianal deficiencies and thrombocytopenia caused by Myelodysplastic syndromes.

27. The antibody product of claim 1, wherein said polyclonal antibody product comprises at least 3 different antibodies.

28. The antibody product of claim 1, wherein said polyclonal antibody product comprises antibodies capable of binding at least 3 distinct epitopes.

29. The antibody product of claim 1, wherein said antibody is of isotype IgG1.

30. The antibody product of claim 1, wherein said antibody is polyvalent.

31. The antibody product of claim 1, wherein the recombinant anti-RhesusD antibody comprises human constant regions.

32. The antibody product of claim 1, wherein the antibodies comprise human variable regions.

33. The antibody product of any claim 1, wherein the antibodies comprise CDR1, CDR2 and CDR3 regions from the VH:VL pairs selected from the CDR1, CDR2 and CDR3 regions described in FIGS. 3 and 4.

34. The antibody product of claim 1, wherein the antibody product is produced by a method that comprises generation of a pWCP containing anti-RhDrpAb with 25 individual monoclonal anti-RhD antibody production cell lines (RhD157, 159, 160, 162, 189, 191, 192, 196, 197, 199, 201, 202, 203, 207, 240, 241, 245, 293, 301, 305, 306, 317, 319, 321, 324).

35. The antibody product of claim 1, wherein the antibody product comprises antibodies with the CDR sequences of the 25 antibodies encoded by clones RhD157, 159, 160, 162, 189, 191, 192, 196, 197, 199, 201, 202, 203, 207, 240, 241, 245, 293, 301, 305, 306, 317, 319, 321, 324.

36. The antibody product of claim 1, wherein the antibody product comprises antibodies with the V_{H}V_{L} sequences of the 25 antibodies encoded by clones RhD157, 159, 160, 162, 189, 191, 192, 196, 197, 199, 201, 202, 203, 207, 240, 241, 245, 293, 301, 305, 306, 317, 319, 321, 324.

37. Use of the antibody product of claim 1 in the manufacture of a medicament for the treatment or prophylaxis of thrombocytopenia, wherein said antibody is prepared for administration in a dose of 10-500 microgram specific antibody/kg patient body mass.

38. A method of treatment of thrombocytopenia in a subject, said method comprising administering to said subject suffering from thrombocytopenia a therapeutically effective amount of a recombinant anti-RhesusD antibody product, wherein said antibody is administered in a dose of 10-500 microgram specific antibody/kg patient body mass.

39. The method of claim 38, wherein said subject suffering from thrombocytopenia also has anaemia.

40. The method of claim 38 or 39, wherein said anti-RhesusD antibody is administered intravenously.

41. The method of claim 38 or 39, wherein said anti-RhesusD antibody is administered subcutaneously.

42. A method of avoiding extravascular haemolysis during anti-RhesusD based treatment in a subject suffering from thrombocytopenia, said method comprising administering to a subject suffering from thrombocytopenia a therapeutically effective amount of a recombinant anti-RhesusD antibody, wherein said antibody is administered in a dose of 10-500 microgram specific antibody/kg patient body mass.

43. The method of claim 42, wherein said subject suffering from thrombocytopenia also has anaemia.

44. A composition for treatment of thrombocytopenia comprising the antibody product of claim 1 and a physiologically acceptable carrier.

45. A composition for treatment of thrombocytopenia comprising the antibody product of claim 1 and a pharmaceutically acceptable carrier.

46. A kit-of-parts for simultaneous, separate or sequential treatment of thrombocytopenia comprising the antibody product of claim 1 and at least one additional component.

47. The kit-of-parts according to claim 46, wherein the additional component is a corticosteroid.

48. The kit-of-parts according to claim 46, wherein the additional component is prednisolone.


SEQUENCE LISTING (260 sequences)

SEQ ID NO: 1 - DNA, 48 bp, Saccharomyces cerevisiae
gaagttcctattccgaagttcctattctctagaaagtataggaacttc

SEQ ID NO: 2 - DNA, 30 bp, Artificial
  Synthetic primer sequence
ggaggcgctcgagacggtgaccagggtgcc

SEQ ID NO: 3 - DNA, 30 bp, Artificial
  Synthetic primer sequence
ggaggcgctcgagacggtgaccattgtccc

SEQ ID NO: 4 - DNA, 30 bp, Artificial
  Synthetic primer sequence
ggaggcgctcgagacggtgaccagggttcc

SEQ ID NO: 5 - DNA, 30 bp, Artificial
  Synthetic primer sequence
ggaggcgctcgagacggtgaccgtggtccc

SEQ ID NO: 6 - DNA, 39 bp, Artificial
  Synthetic primer sequence
ccagccggggcgcgcccagrtgcagctggtgcartctgg

SEQ ID NO: 7 - DNA, 39 bp, Artificial
  Synthetic primer sequence
ccagccggggcgcgccsaggtccagctggtrcagtctgg

SEQ ID NO: 8 - DNA, 39 bp, Artificial
  Synthetic primer sequence
ccagccggggcgcgcccagrtcaccttgaaggagtctgg

SEQ ID NO: 9 - DNA, 39 bp, Artificial
  Synthetic primer sequence
ccagccggggcgcgccsaggtgcagctggtggagtctgg

SEQ ID NO: 10 - DNA, 39 bp, Artificial
  Synthetic primer sequence
ccagccggggcgcgccgaggtgcagctggtggagwcygg

SEQ ID NO: 11 - DNA, 39 bp, Artificial
  Synthetic primer sequence
ccagccggggcgcgcccaggtgcagctacagcagtgggg

SEQ ID NO: 12 - DNA, 39 bp, Artificial
  Synthetic primer sequence
ccagccggggcgcgcccagstgcagctgcaggagtcsgg

SEQ ID NO: 13 - DNA, 39 bp, Artificial
  Synthetic primer sequence
ccagccggggcgcgccgargtgcagctggtgcagtctgg

SEQ ID NO: 14 - DNA, 39 bp, Artificial
  Synthetic primer sequence
ccagccggggcgcgcccaggtacagctgcagcagtcagg

SEQ ID NO: 15 - DNA, 51 bp, Artificial
  Synthetic primer sequence
accgcctccaccggcggccgcttattaacactctcccctgttgaagctctt

SEQ ID NO: 16 - DNA, 38 bp, Artificial
  Synthetic primer sequence
caaccagcgctagccgacatccagwtgacccagtctcc

SEQ ID NO: 17 - DNA, 38 bp, Artificial
  Synthetic primer sequence
caaccagcgctagccgatgttgtgatgactcagtctcc

SEQ ID NO: 18 - DNA, 38 bp, Artificial
  Synthetic primer sequence
caaccagcgctagccgaaattgtgwtgacrcagtctcc

SEQ ID NO: 19 - DNA, 38 bp, Artificial
  Synthetic primer sequence
caaccagcgctagccgatattgtgatgacccacactcc

SEQ ID NO: 20 - DNA, 38 bp, Artificial
  Synthetic primer sequence
caaccagcgctagccgaaacgacactcacgcagtctcc

SEQ ID NO: 21 - DNA, 38 bp, Artificial
  Synthetic primer sequence
caaccagcgctagccgaaattgtgctgactcagtctcc

SEQ ID NO: 22 - DNA, 50 bp, Artificial
  Synthetic primer sequence
accgcctccaccggcggccgcttattatgaacattctgtaggggccactg

SEQ ID NO: 23 - DNA, 50 bp, Artificial
  Synthetic primer sequence
accgcctccaccggcggccgcttattaagagcattctgcaggggccactg

SEQ ID NO: 24 - DNA, 38 bp, Artificial
  Synthetic primer sequence
caaccagcgctagcccagtctgtgctgactcagccacc

SEQ ID NO: 25 - DNA, 38 bp, Artificial
  Synthetic primer sequence
caaccagcgctagcccagtctgtgytgacgcagccgcc

SEQ ID NO: 26 - DNA, 38 bp, Artificial
  Synthetic primer sequence
caaccagcgctagcccagtctgtcgtgacgcagccgcc

SEQ ID NO: 27 - DNA, 36 bp, Artificial
  Synthetic primer sequence
caaccagcgctagcccartctgccctgactcagcct

SEQ ID NO: 28 - DNA, 41 bp, Artificial
  Synthetic primer sequence
caaccagcgctagccctttcctatgwgctgactcagccacc

SEQ ID NO: 29 - DNA, 41 bp, Artificial
  Synthetic primer sequence
caaccagcgctagccctttcttctgagctgactcaggaccc

SEQ ID NO: 30 - DNA, 38 bp, Artificial
  Synthetic primer sequence
caaccagcgctagcccacgttatactgactcaaccgcc

SEQ ID NO: 31 - DNA, 38 bp, Artificial
  Synthetic primer sequence
caaccagcgctagcccaggctgtgctgactcagccgtc

SEQ ID NO: 32 - DNA, 41 bp, Artificial
  Synthetic primer sequence
caaccagcgctagcccttaattttatgctgactcagcccca

SEQ ID NO: 33 - DNA, 38 bp, Artificial
  Synthetic primer sequence
caaccagcgctagcccagrctgtggtgacycaggagcc

SEQ ID NO: 34 - DNA, 38 bp, Artificial
  Synthetic primer sequence
caaccagcgctagcccwgcctgtgctgactcagccmcc

SEQ ID NO: 35 - DNA, 381 bp, Homo Sapiens
gaggtgcagctggtggagtctgggggaggcttggtacagcctggagggtccctgagactctcctgtgtagcctctggattcaccttcaggagttttgacatgaactgggtccgccaggctccagggaaggggctggagtgggtttcatacattaatagtaggggtagtaccatatactacgcagactctgtgaagggccgattcaccatctccagagagaacgccaagaactcactgtatctgcaaatgaacagcctgagagccgaggacacggctgtttattactgtgcgagagatttgtacggtgactatgaccctaagtcctactattactacggtatgggcgtctggggccaagggaccacggtcaccgtctcgagt

SEQ ID NO: 36 - DNA, 375 bp, Homo Sapiens
gaggtgcagctggtggagaccgggggaggcgtggtccagcctgggaggtccctgagactctcctgtgcagcgtctggattctccttcagtaactttggcttccactggatccgccagtctccaggcaaggggctcgaatgggtggcagttatttggtatgatggaagcaacagattctatgcagattccgtgaagggccgattcaccatctccagagatagttcgaagaacatgctgtttctgcaaatggacagcctgagagccgaggacacggctgtgtattactgtgcgagagaaatttccatgaaagtagtgatccgcagacactacgttatggacgtctggggccacgggaccacggtcaccgtctcgagt

SEQ ID NO: 37 - DNA, 381 bp, Homo sapiens
gaggtgcagctggtggagaccgggggaggcttagtacagcctggagggtccctgagactctcctgtgcagcctctggattcagcttcaggagttatgacatgaactgggtccgccaggctccagggaaggggctggagtgggtgtcatacatcagtggcagaggtagtacaacatattacgcagactctgtgaagggccgattcaccatctccagagacaacgccaagaactcactgtatctgcaaatgaacagtctgagagccgaggacacggctgtttattactgtgcgagagatttgtacggtgactacgatcctaagtcctactattactacgctatggacgtctggggccacgggaccacggtcaccgtctcgagt

SEQ ID NO: 38 - DNA, 375 bp, Homo Sapiens
gaggtgcagctggtggagtctgggggaggcgtggtccagcctgggaggtccctgagactctcctgtgcagcgtctggattcaccttcagtaattatggcatgcactgggtccgccaggctccaggcaaggggctggagtgggtggcggttatatggtatgatggaagtaatagattctatgcggactccgtgaagggccgattcaccatctccagagacaattccaagaatacgctgtatctgcaaatgaacaacctgagagccgaggacacggctctctattcctgtgcgagagagattactacgacagtagtggtccgaagacactaccttatggacatctggggccaagggaccacggtcaccgtctcgagt

SEQ ID NO: 39 - DNA, 378 bp, Homo Sapiens
gaggtgcagctggtggagtctgggggaggcgtggtccagtctgggaggtccctgagactctcatgtgcagcctctggattcaccttcagtaacaatggcatgcactgggtccgccaggctccaggcaaggggctggagtgggtggcatttatttggtatgatggaagtaataaatactatgcagactccgtgaagggccgattcaccatctccagagacaattccaagaacacactgtatctgcaaatggacggcctgagagccgaggactcggctgtgtattactgtgcgagagaggaaatagcagctcgtctttattctcgctaccactacgctatggacgtctggggccaagggacaatggtcaccgtctcgagt

SEQ ID NO: 40 - DNA, 375 bp, Homo Sapiens
caggtgcagctggtggagtctgggggaggcctggtcaagcctggggggtccctgagactctcctgtgcagcctctggattcggcttcagtgcttatagcatgaactgggtccgccaggctccggggaaggggctggagtgggtctcatccattactagcactactacatactacgcagactcagtgaagggccgattcagcatctccagagacaacgccaagagcacactgtacctgcgaatgaacagcctgagagccgaggacacggctgtatattattgtgtgagagaaatcgcctttagggggagcacttattctcggtggtcgtactactttgacttctggggccagggaaccctggtcaccgtctcgagt

SEQ ID NO: 41 - DNA, 384 bp, Homo Sapiens
caggtgcagctgcaggagtcgggcccaggactggtgaagccttcggagaccctgtccctcacctgcactgtctctggtggctccatcagtagttactactggagctgggtccggcagcccccagggaagggactggagtggattgggtatatctattacagtgggagcaccaactacaacccctccctcaagagtcgagtcaccatagcattagacacgtccaagaaccagttctccctgaagctgaggtctgtgaccgctgcggacacggccgtgtatttctgtacgagagactggaggcaatatgggtcggcgatccgaggttctcgatactactacgggatggacgtctggggccaagggaccacggtcaccgtctcgagt

SEQ ID NO: 42 - DNA, 387 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagacatggttactatggttcggggagcctacagaaactactactactacggtatggacgtctggggcaaagggaccacggtcaccgtctcgagt

SEQ ID NO: 43 - DNA, 381 bp, Homo Sapiens
gaggtgcagctggtggagaccgggggaggcgtggtccagcctgggaggtccctgagactctcctgtgcagcctctggattcagcttcagtaactatggcatgcactgggtccgccaggctccaggcaaggggttggagtgggtggcagttatttggtttgatggaagtattaaatattatgtagactccgtgaaaggccgattcaccatctccagagacaattccaagaacacactctatctgcaaatgaacagcctgagagccgaggagacggctatatatttctgtgcgagagaaaatagtgttctagtcccaggtactatacggaggcgatattatttggactactggggccagggaaccctggtcaccgtctcgagt

SEQ ID NO: 44 - DNA, 381 bp, Homo Sapiens
caggtgcagctggtggagtctgggggagacctggtacagcctggagggtccctgagactctcctgtgcagcctctggattcaccttcaggagttatgaaatgaactgggtccgccaggctccagggaaggggctggagtgggtttcatacattaatagtagaggtaataccaaatactacgcagactctgtgaagggccgattcaccatctccagagacaacgccaagaagtcactgtacctgcaaatgagcagcctgagagccgaggacacggctgtttattactgtgcgagaaatttgttcggtgactacgatcttaagtcctactactataacgctatggacgtctggggccaaggcaccctggtcaccgtctcgagt

SEQ ID NO: 45 - DNA, 381 bp, Homo Sapiens
caggtgcagctggtggagtctgggggaggcctggtcaagcctggggggtccctgagactctcctgtgcagcctctggattcaccttcagtagttatgccatgaactgggtccgccaggctccagggaagggactggagtgggtctcatccattagcggtactagtagttacatatactatgcagactcagtgaagggccgatttaccattttcagagacaacgccaagagctcagtttatctgcaaatgaacagcctgagagtcgaggacacggctgtctattactgcgcgagagatagatggtggggcatggttcggagagtttttcccacctatccctttgactactggggccagggaaccctggtcaccgtctcgagt

SEQ ID NO: 46 - DNA, 375 bp, Homo Sapiens
caggtgcagctggtggagaccgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaagactatgcagaccccgtgaagggccgattcaccatctccagagacaattccaagaacacgctgtatctgcaaatgaacagcctcagagccgaggacacggctgtgtattactgtgcgagagagatcgcctcccgtggatatagtcgctacttatactactttgactcctggggccagggaaccctggtcaccgtctcgagt

SEQ ID NO: 47 - DNA, 387 bp, Homo Sapiens
caggtcaccttgaaggagtctggtcctgtgctggtgaaacccacagagaccctcacgctgacctgcaccgtctctgggttctcactcagcaatgctagaatgggtgtgagctggatccgtcagcccccagggaaggccctggagtggcttgcacacattttttcgaatgacgaaaaatcctacagcacatctctgaagagcaggctcaccatctccaaggacacctccaaaagccaggtggtccttaccatgaccaacatggaccctgtggacacagccacatattactgtgcacggatgaggcttactatggttcggggagttattacgtactactactacagtatggacgtctggggccaagggaccacggtcaccgtctcgagt

SEQ ID NO: 48 - DNA, 381 bp, Homo Sapiens
caggtgcagctgcaggagtcgggcccaggactggtgaagccttcacagaccctgtccctcacctgcactgtctctggtggctccatcagcagtggtagttactactggagctggatccggcagcccgccgggaagggaccggagtggattgggcgtatctataccagtgggagtaccaactacaacccctccctcaagagtcgactcaccatatcagtagacacgtccaagaaccagttctccctgaagctgacctctgtgaccgccgcagacacggccgtgtattactgtgcgagggccccttcttactatgatagtagtggttatcgttactggtacatcgatctctggggccgtggcaccctggtcaccgtctcgagt

SEQ ID NO: 49 - DNA, 378 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaagactatgcagactccgtgaagggccgattcaccatctccagagacaactccaagaacacgctgtatctgcaaatgaacagcgtgagagccgaggacacggctgtttattactgtgcgagagaattgagcacgcaacgtggatacagccgctaccactatgttatggacgtctggggccaagggaccacggtcaccgtctcgagt

SEQ ID NO: 50 - DNA, 363 bp, Homo Sapiens
caggtgcagctggtggagtctgggggaggcgtggtccagcctgggaggtccctgagactctcctgtgcagcgtctggattcaccttcagtaactatggcatgcactgggtccgccaggctcctggcaaggggctggaatgggtggcagttatctggtttgatggaagtaatagagactatgcagactccgtgaagggccgattcaccatctccagagacaattccaagaagacgctctatctgcaaatgaacagcctgagagccgaggacacggctgtttattactgtgcgacagagttggccagagggcggctacgagccctagagtactggggccagggaaccctggtcaccgtctcgagt

SEQ ID NO: 51 - DNA, 378 bp, Homo Sapiens
caggtgcagctggtgcagtctgggggaggcgtggtccagcctgggaagtccctgagactctcctgtgcagcgtctggattcaccttcagtacctatggcatgcactgggtccgccaggctccaggcaaggggctggagtgggtggcagttatatggtatgatggaagtaataaatactatgcagactccgtgaagggccgattcaccatctccagagacacttccaagaacacgctgtatctgcaaatgaacagcgtgagagtcgaggacacggctgtttattactgtgcgagagatttgaccacgcaacgtggatacagccgctatcactatgttatggacgtctggggccaagggaccacggtcaccgtctcgagt

SEQ ID NO: 52 - DNA, 378 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgacggaagtaataagtactatgcagactccgtgaagggccgattcaccatctccagagacaattccaagaacacgctgcatctgcaaatgaacagcctgagagccgaggacacggctgtgtattactgtgcgagagaagtgggttttggcagtggctggtcacgatactactacggtatggacgtctggggccaagggaccacggtcaccgtctcgagt

SEQ ID NO: 53 - DNA, 390 bp, Homo Sapiens
gaggtgcagctggtggagtctggaggaggaggcgtggtccagcctgggaggtccctgaggctctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagctgaggacacggctgtgtattactgtgcgagagagagtactctgtatagcagcagctggtacaggaggtactactactacggtatggacgtctggggccaagggaccacggtcaccgtctcgagt

SEQ ID NO: 54 - DNA, 387 bp, Homo Sapiens
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagagagtactctatatagcagcagctggtacaggaggtactactactacagtatggacgtctggggccaagggaccacggtcaccgtctcgagt

SEQ ID NO: 55 - DNA, 384 bp, Homo Sapiens
caggtgcagctggtggagtctgggggaggcgtggtccagcctgggaggtccctgagactctcctgtgcggcgtctggattcaccttcagtagctatggcatgcactgggtccgccaggctccaggcaaggggctggagtgggtggcagttatatggttcgatggaagtaatagatactatggagactccgtgaagggccgagtcaccatctccagagacaattccaagaacacgctgtatctgcgaatgaacagcctgagagccgaggacacggctgtgtattactgtgcgagagagttctatacacgtagcgggctatggtcacaagggtactcctattacatggacgtctggggcaaagggaccacggtcaccgtctcgagt

SEQ ID NO: 56 - DNA, 387 bp, Homo Sapiens
gaggtgcagctggtggagtctgggggaggcgtggtccagcctgggaggtccctgagactctcctgtgcagcgtctggattcaccttcagtaactatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagagatggtctcctatagcagcagctggtaccgccgctactactactacgttatggacgtctggggcaaagggaccacggtcaccgtctcgagt

SEQ ID NO: 57 - DNA, 369 bp, Homo Sapiens
gaggtgcagctggtggagtctgggggaggcgtggtccagcctgggaggtccctgagactctcctgtgcagcctctggattcagcttcagtaactatggcatgcactgggtccgccaggctccaggcaaggggctggagtgggtggcagttatatcatatgatggaagtgagaagtactatgcagactccgtgaagggccgattcaccatctccagagacaattccaagaaggcgctgtatctgcaaatgaacagcctgagagctgaggacacggctatgtattattgtaagaataaagtgggagctacccggcgggcagtcgttgcttttgatatctggggccaagggaccacggtcaccgtctcgagt

SEQ ID NO: 58 - DNA, 381 bp, Homo Sapiens
gaggtgcagctggtggagaccgggggaggcctggtcaagcctggggggtccctgagactctcctgtgcagcctctggattcaccttcaggagttatagcatgaactgggtccgccaggctccagggaaggggctggagtgggtctcatccattggtagtagtagtacttacacatactccgcagactcagtgaagggccgattcaccatctccagagacaacgccaagaactcactgtatctgcaaatgaacagcctgagagccgaggacacggctgtgtattactgtgcgagaggagagcctctaaactatgattacatttggggagggtatcgtttcactatccactggggccagggaaccctggtcaccgtctcgagt

SEQ ID NO: 59 - DNA, 387 bp, Homo Sapiens
caggtgcagctggtggagtctgggggaggcgtggtccagcctgggaggtccctgagactctcctgtgcagcgtctggattcaccttcagtagttatggcatgcactgggtccgccaggctccaggcaaggggctggaatgggtggcaattatatggtttgatggaagtaataaatactatgcagactccgtgaagggccgattcaccatctccagagacaattccaagaacacgctgtatctgcaaataaacagcctgagagctgaggacacggctgtgtattactgtgcgaaagagcacggctattatagcagcagctggtaccgaaactactattactacgctatggacgtctggggccaagggaccacggtcaccgtctcgagt

SEQ ID NO: 60 - DNA, 363 bp, Homo Sapiens
gaggtgcagctggtggagtccgggggaggcgtggtccagcctgggaggtccctgagactctcctgtgcggcgtctggattcaccttcagtagctatggcatgcactgggtccgccaggctccaggcaaggggctggagtgggtggcagttatatggtatgatggaagtaataaagactatgtagactctgtgaagggccgattcaccatctccagagacaattccaagaacacgctgtatctgcaaatgaacagtctgagagccgaggacacagctgtgtattactgtgcgagagagttggccaaagggcggctacgagacctagaccactggggccagggaaccctggtcaccgtctcgagt

SEQ ID NO: 61 - DNA, 387 bp, Homo Sapiens
gaggtgcagctggtggagtctgggggaggcgtggtccagcctgggaggtccctgagactctcctgtgcagcctctggattcaccttcaggacttctgccatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagagatggtctcctatagcagcagctggtaccgccgctactactactacaatatggacgtctggggcaaagggaccacggtcaccgtctcgagt

SEQ ID NO: 62 - DNA, 375 bp, Homo Sapiens
caggtgcagctggtggagtctgggggaggcgtggtccagcctgggacgtccctgagattgtcctgtgcagcgtctgggttcacctttagaacctatggcatgcactgggtccgccaggctccaggcaaggggctggagtgggtggcatatatatggtatgatggaagtaataaatactatgcagactccgtgaagggccgattcaccatctccagagacaattccaagagcacgctgaatctgcaaatgaacagcctcagagccgaggacacggctgtgtattactgtgcgagagagatcgcctcccgtggatatagtcgctacttatactactttgactcctggggccagggcaccctggtcaccgtctcgagt

SEQ ID NO: 63 - DNA, 387 bp, Homo Sapiens
cagctgcagctgcaggagtcgggcccaggactggtgaagccttcggggaccctgtccctcacctgcgctgtctctggtggctccatgaggagtagtaactggtggacttgggtccgccagcccccagggaaggggctggaatggattggggaaatccatcatggtgggagcaccaactacaacccgtccctccagagtcgagtcacgatatcagtagacaagtccaagaaccggttctccctgaagctgagctctgtgaccgccgcggacacggccgtatatcactgtgcgagggggaggagttattatgatagtagtgggcattcctttcgcggtctggtaccttttgatatctggggccaagggacaatggtcaccgtctcgagt

SEQ ID NO: 64 - DNA, 384 bp, Homo Sapiens
caggtgcagctgcaggagtcgggcccaggactggtgaagccttcggagaccctgtccctcatctgcactgtctctggtggctccatcagtagtaactactggagctggatccggcagcccccagggaagggactggagtggattgggtatatctattacagtgggaacaccaactacaacccctccctcaagagtcgagtcaccatatcattagacacgtccaagaaccagttctccctgaagctgagatctgtgaccgctgcggacacggccgtgtattactgtgcgagagagtggaggcagtatggctcggggatccgaggttctcgatactactacggtatggacgtctggggccagggcaccctggtcaccgtctcgagt

SEQ ID NO: 65 - DNA, 387 bp, Homo Sapiens
gaggtgcagctggtggagtctgggggaggcgtggtccagcctgggaggtccctgagactctcctgtgcagcgtctggattcaccttcagtaaccatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagggccgaggacacggctgtgtattactgtgcgagagagatggcctcctatagcagcagctggtaccgccgctactactactacgttatggacgtctggggcaaagggaccacggtcaccgtctcgagt

SEQ ID NO: 66 - DNA, 375 bp, Homo Sapiens
caggtgcagctggtggagtctgggggaggcgtggtccagcctgggaggtccctgagactctcctgtgcagcgtctggattcaccttcagtacctatggcatgcactgggtccgccaggctccaggcaaggggctggagtgggtggcagttatatggtatgatggaagtcagaaatactatgtagactccgtgaagggccgattcaccatctccagagacaattccaagaacacgctgtatctgcaaatgaacagcctgagagccgaggacacggctgtctattactgtgcgagagaggttgcggttcggggagttattcgctactactacggtatggacgtctggggccaagggaccacggtcaccgtctcgagt

SEQ ID NO: 67 - DNA, 384 bp, Homo Sapiens
caggtgcagctggtggagtctgggggaggcgtggtccagcctgggaggtctctgagactctcctgtgcagcctcgggattcagcttcagtagttatggcatgcactgggtccgccaggctccaggcaagggactggagtgggtggcaattatttggtatgatgggagtaacaaactctatgcagactccgtgaagggccgattcaccatctccagagacaattccaggaatacgttgtatctgcaaatgagcagtgtgagagccgaggacacggctgtgtattactgtgcgagagactctgttcggggagtcagtagatgggggactcagaaatattacgctatggacgtctggggccaagggaccacggtcaccgtctcgagt

SEQ ID NO: 68 - DNA, 378 bp, Homo Sapiens
gaggtgcagctggtgcaatctgggggaggcgtggtccagcctgggaggtccccgagactctcctgtgcagcgtctggattcaccttcagtagctatggcatgcactgggtccgccaggctccaggcaaggggctggagtgggtggcatttatatggtatgatggaagtaataaatactatgcagactctgtgaagggccgattcaccatctccagagacaattccaagaacacgctgtatctgcaaatgaacagcctgagagccgaggacacggctgtgtattactgtgcgagagatctccggaaccacgttttttggagtggttattctacctcttttgactactggggccagggaaccctggtcaccgtctcgagt

SEQ ID NO: 69 - DNA, 387 bp, Homo Sapiens
gaggtgcagctggtggagaccgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagagatggtctcctatagcagcagctggtaccgccgctactactactacaatatggacgtctggggcaaagggaccacggtcaccgtctcgagt

SEQ ID NO: 70 - DNA, 387 bp, Homo Sapiens
caggtgcagctggtggagaccgggggaggcgtggtccagcctgggaggtccctgagactctcctgtgcagcgtctggattcaccttcagtagctatggcatgcactgggtccgccaggctccaggcaaggggctggagtgggtggcagttatatggtctgatggaagtaataaatactatgcagactccgtgaagggccgattcaccatctccagagacaattccaggaacacgctgtatctgcaaatgaacagcctgagagccgaggacacggctgtgttttactgtgcgagagagcaaggggggtatagcagcagttggtaccgccgctactactactactatatggacgtctggggccaagggaccacggtcaccgtctcgagt

SEQ ID NO: 71 - DNA, 375 bp, Homo Sapiens
gaggtgcagctggtggagtctgggggaggcgtggtccagcctggaaggtccctgagactctcctgtgcagcgtctggattcaccttcaatacctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagaggttgcggttcggggagttattcgctactactacgctatggacgtctggggccaagggaccacggtcaccgtctcgagt

SEQ ID NO: 72 - DNA, 369 bp, Homo Sapiens
caggtgcagctggtggagtctgggggaggcgtggtccagcctgggaggtctctgagactctcctgtgcagcctctggattcagcttcagtaactatggcatgcactgggtccgccaggctccaggcaaggggctggagtgggtggcagttatatcatatgatggaagtgagaagtactatgcagactccgtgaagggccgattcaccatctccagagacaattccaagaaaacgctgtatctgcaaatgaacagcctgagagctgaggacacggctatgtattattgtaagaataaagtgggagctacccggcgggcagttgttgctgttgatatttggggccaagggacaatggtcaccgtctcgagt

SEQ ID NO: 73 - DNA, 381 bp, Homo Sapiens
caggtgcagctggtggagtctgggggaggcttggtacagcctggagggtccctgagactctcctgtgtaggctctggattcaccttcaggagttttgatatgaactgggtccgccaggctccagggaaggggctggagtggatttcatatattaatagtagggggaacaccagatactatgtagactctgtgaagggccgattcaccatctccagagacaacgccaagaactcattgtatctgcaaatgaacagcctgcgagccgaggacacggctgtttactactgtgcgagagatttgtacggtgactacgatcctaagtcctactattactacggtatggacgtctggggccaagggacaatggtcaccgtctcgagt

SEQ ID NO: 74 - DNA, 375 bp, Homo Sapiens
gaggtgcagctggtggagtctgggggaggcgtggtccagcctgggaggtccctgagactctcctgtgaagcgtctggattcaccttcagtaattatggcatgcactggttccgccaggctccaggcaaggggctggagtgggtggcaattatatggtatgatggaagtaataaacactatgcagactccgtgaagggccgattcaccatctccagagacaattcgaagaacacgctgtatctgcaaatgaacagcctcagagccgaggacacggctgtgtatttctgtgcgagagagatcgcctcccgtggatatagtcgctacttatactactttgactcctggggccagggaaccctggtcaccgtctcgagt

SEQ ID NO: 75 - DNA, 375 bp, Homo Sapiens
caggtgcagctggtggagtctgggggaggcgtggtccagcctgggaggtccctgagactctcctgtgcagcgtctggattcaccttcaggacctatggcatgcactgggtccgccaggctccaggcaaggggctggagtgggtggcagttatatggtatgatggaagtaataaagactatgcagactccgtgaagggccgattcaccatctccagagacaattccaagaacacgctgcatctgcaaatgaacagcctgagagccgaggacacggctgtgtattactgtgcgagagagatcgcctcccgtggatatagtcggtacttatactaccttgacttctggggccagggcaccctggtcaccgtctcgagt

SEQ ID NO: 76 - DNA, 378 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatattatgcagactccgtgaagggccgattcaccatctccagagacaattccaagaacacactatatctgcaaatgaacagcgtgagagccgaggacacggctgtttattactgtgcgagagatttgagcacgcaacgtggatacagccgctactactatgttatggacgtctggggccaagggaccacggtcaccgtctcgagt

SEQ ID NO: 77 - DNA, 363 bp, Homo Sapiens
caggtgcagctggtggagtctgggggaggcgtggtccagcctgggaggtccctgagactctcctgtgcagcgtctggattcaccttcagtaactttggcatgcactgggtccgccaggctccaggcaaggggctggagtgggtggcagttatatggtatgatggaagtaataaagactatgcagactccgtgaagggccgattcaccatctccagagacaattccaagaggacgctgtatctgcaaatgaacagcctgagagccgaggacacggctgtgtattactgtgcgagagagttggccagagggcggctacgagacctagactactggggccagggcaccctggtcaccgtctcgagt

SEQ ID NO: 78 - DNA, 384 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagagttctatacacgtagcgggctatggtcacaagggtactcctattacatggacgtctggggcaaagggaccacggtcaccgtctcgagt

SEQ ID NO: 79 - DNA, 387 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtttgatggaggtaataaatactatgcagactccgcgaagggccgattcaccatctccagagacaattccaagaacacgctgtatctgcaaatgaacagcctgagagccgaggacacggctgtgtattactgtgcgagagatgcgtcggtgctttctggattggttactcgaaggttagtctactacggtatggacgtctggggccaagggaccacggtcaccgtctcgagt

SEQ ID NO: 80 - DNA, 387 bp, Homo Sapiens
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcgtttatatcatatgatggaagtaataaatactatgcagactccgtgaagggccgattcaccatctccagagacaattccaagaacacgctgtctctgcaaatgaacagcctgagagctgaggacacggctgtgtattactgtgcgaaagagcacggctattatcgcagcagctggtaccgaaactactactactatgggatggacgtctggggccaagggaccacggtcaccgtctcgagt

SEQ ID NO: 81 - DNA, 387 bp, Homo Sapiens
caggtgcagctggtggagtctgggggaggcgtggtgcagcctgagaggtccctgagactctcctgtgcagcctctggattcaccttcagttcctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatgaagtggggtatagcagcagctggtacaggcgctactactactacgctatggacgtctggggccaagggaccacggtcaccgtctcgagt

SEQ ID NO: 82 - DNA, 384 bp, Homo Sapiens
caggtgcagctggtgcagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagagacagtggtggtagctgccaaaatacgaaaccactactactacgctatggacgtctggggccaagggaccacggtcaccgtctcgagt

SEQ ID NO: 83 - DNA, 387 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcaggtatatggtatgatggaagtaataaatactatgcagactccgtgaagggccgattcaccatctccagagacaattccaggaacacgatgtatctgcaaatgaacagcctgagagccgaggacacggctgtgtattactgtgcgagagagcaaggggggtatagcagcagttggtaccgccgctactactactacaatatggacctctggggccaagggaccacggtcaccgtctcgagt

SEQ ID NO: 84 - DNA, 387 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacaccctgagagacgacgacacggctgtgtattactgtgcgagagagggtactctgtatagcagcagctggtacaggaggtactactactacggtatggacgcctggggccaagggaccacggtcaccgtctcgagt

SEQ ID NO: 85 - DNA, 387 bp, Homo Sapiens
caggtgcagctggtggagactgggggaggcgtggtccagcctgggaggtccctgagactctcctgtgcagcgtctggattcaccttcagtagctatggcatgcactgggtccgccaggctccaggcaaggggctggagtgggtggcaggtatatggtatgatggaagtaataaatactatggagactccgtgaagggccgattcaccatctccagagacaattccaggaatacgctgtatctgcatatgaacagcctcagagccgacgacacggctgtgtattactgtgcgagggaacacggcgggtctaggagtggctggtacactttacgtctcgcgtactactttgactactggggccagggcaccctggtcaccgtctcgagt

SEQ ID NO: 86 - DNA, 387 bp, Homo Sapiens
caggtgcagctgcaggagtccggcccaggactggtgaagccttcggggaccctgtccctcacctgcgctgtctctggtggctccatcaggggtagtaattggtggagttgggtccgccagcccccagggaaggggctggagtggattggggaaatccatcatggtgggagcaccaactacaacccgtccctcaagagtcgagtcacgatatcagtagacaagtccaagaaccggttctccctgaagctgagctctgtgaccgccgcggacacggccgtgtattactgtgcgagggggacgtcttattatgatagtagtggttattcctttcgcggtctggtagcttttgatatctggggccaagggacaatggtcaccgtctcgagt

SEQ ID NO: 87 - DNA, 387 bp, Homo Sapiens
caggtgcagctggtggagtctgggggaggcgtggtccagcctgggaggtccctgagactctcctgtgcagcgtctggattcaccttcagtacctatggcatgcactgggtccgccaggctccaggcaaggggctggagtgggtggcagttatatggtatgatggaagtaataaaaactatgcagactccgtgaagggccgattcaccatctccagagacaattccaagaacacgctgtatctgcaaatgaacagcctgagagccgaggacacggctgtgtattactgtgcgagagacctacaagggtatagaagcagctggtaccggatgtactactactacggtatggacgtctggggccaagggaccacggtcaccgtctcgagt

SEQ ID NO: 88 - DNA, 381 bp, Homo Sapiens
gaggtgcagctggtggagtctgggggaggcctggtcaagcctggggggtccctgagactctcctgtgcagcctctggattcaccttcaggagttatagcatgaactgggtccgccaggctccagggaaggggctggagtgggtctcatccattggtagtagtagtatttacacatactccgcagactcagtgaagggccgattcaccatctccagagacaacgccaagaactcactgtatctgcaaatgaacagcctgagagccgaggacacggctgtgtattactgtgcgagaggagagcctctaaactatgattacatttggggaaggtctcgtctcactatccactggggccagggaaccctggtcaccgtctcgagt

SEQ ID NO: 89 - DNA, 387 bp, Homo Sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatattatgcagactccgtgaagggccgattcaccatctcccgagacaattccaagaacacgctgtatctgcaaatgaacagcctgagagctgaggacacggctgtgtattactgtgcgagagattgggtcactcgcagcagcaactggtacaggaactactactactacggtatggacgtctggggccaagggaccacggtcaccgtctcgagt

SEQ ID NO: 90 - DNA, 372 bp, Homo Sapiens
gaggtgcagctggtggagtctgggggaggcttagttcagccggggaggtccctgagactctcctgtgtagcctctggattcaccttcagtagctactggatgcactgggtccgccaagttccagggaaggggctggtgtgggtctcacgtattaatgttgatgggaagagcacaagctacgcggactccgtgaagggccgattcaccatctccagagacaacgccaagaacacgctgtacctgcaaatgaacagtctgagagccgaggacacggctgtgtattactgtgcaagagatccccgacgatttttggagtgggcccgctacggtatggacgtctggggccgagggaccacggtcaccgtctcgagt

SEQ ID NO: 91 - DNA, 654 bp, Homo Sapiens
gaaattgtgttgacgcagtctccagccaccctgtctttgtctccaggggagagagccaccctctcctgcagggccagtcagagtgttagcagctacttagcctggtaccaacagaaacgtggccaggctcccaggctcctcatctttaatgcatccaacagggccactggcatcccagccaggttcagtggcagtgggtctgggacagacttcactctcaccatcagcagcctagagcctgaagattttgcagtttattactgtcagcagcgtagcagctggcctccgatgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 92 - DNA, 648 bp, Homo Sapiens
gacatccagatgacccagtctccatcccccctgtctgcatctgtaggagacagagtctccatcacttgccgggcgagtcggggcattagcaattctttagcctggtatcagcagaaaccagggaaagttcctaagctcctcatctatgctgcatccactttgcaatcaggggtcccatctcggttcagtggcggtggatctgggacagatttcactctcaccatcagcagcctgcagcctgaagatgttgcaacttattactgtcacacgtataacagtgcccccttcgctttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 93 - DNA, 654 bp, Homo Sapiens
gaaattgtgatgacacagtctccaggcaccctgtctttgtctccaggggaaagagccaccctctcctgcagggccagtcacactgtcagcagcggctacttagcctggtatcagcagaaacctggccaggctcccaggctcctcatctatggtgcatccaacagggccactggcgtcccagacaggttcggtggcagtgggtctgggacagacttcactctcaccatcagcagactggagcctgaagattctgcagtgtatttttgtcagcaatatggaacctcaccgggggtcactttcggccaagggacacgactggaaattgaacgaactgtggctgcaccatctgtcttcatcttcccgccatctgatgagcagttgaaatctgggactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 94 - DNA, 648 bp, Homo Sapiens
gacatccagttgacccagtctccatcctccctgtctgcatctgtaggagacagagtctccatcacttgccgggcgagtcagggcattagcaattctttagcctggtatcagcagaaaccagggaaagttcctaagctcctcatctatgctgcatccactttgcaatcaggggtcccatctcggttcagtggcggtggatctgggacagatttcactctcaccatcagcagcctgcagcctgaagatgttgcaacttattactgtcaaaagtataacagtgcccccttcgctttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 95 - DNA, 651 bp, Homo Sapiens
gacatccagatgacccagtctccatccttcctgtctgcatctgtaggagacagagtcaccatcacttgccgggccagtcagggcattagacgttatttagcctggtttcagcaaaaaccagggaaagcccctaaactcctgatcttttctgcatccactttgcaaagtggggtcccatcaaggttcagcggcagtggatctgggacagaattcactctcacaatcagcagcctgcagcctgaggattttgcaacttattactgtcaacagcttagtagttaccctccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 96 - DNA, 648 bp, Homo Sapiens
gacatccagttgacccagtctccatcttctgtgtctgcttctgtaggggacagcgttaccatcacttgtcgggcgagtcaggctgtgagcgggtgggtagcctggtatcagcagaaaccagggaaagcccctaaactcctgatctttggtttgtccaatttggaggatggggtcccatcaaggttcagcggcagtggatctgcgacagacttcactctcaccatcaccggcctgcagcctgaagatttggcaacgtactactgtctacaggctaacaggttccccctctctttcggcggagggaccagggtagagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 97 - DNA, 654 bp, Homo Sapiens
gaaattgtgttgacgcagtctccaggcaccctgtctttgtctccaggggaaagagccaccctctcctgcagggccagtcagaatcctagacgcaactttttagcctggtaccaacagaaacctggccaggctcccaggcttctcatctatgctgcatccaccagggccaccggcatcccagacaggttcagtggcagtgggtctgggacagacttcactctcaccatcgacagactggagcctgaagattctgcagtgtattactgtcaggtctatggtagctcacctctgtacacttttggccaggggaccaaggtggagatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 98 - DNA, 654 bp, Homo Sapiens
gaaattgtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgaccctgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 99 - DNA, 648 bp, Homo Sapiens
gacatccagttgacccagtctccatcctccctgtctgcatctgtgggagacagagtcaccatcacttgccgggcaagtcagagcattagcagctatttaaattggtatcagcagaaaccaggggaagcccccaaactcctgatctatgttgcatccactttgcaaagtggggccccatcaaggttcagtggcagtggatctgggacagattacactctaaccattagcagtctgcaacctgaagattctgcaactttctactgtcaacagacttacagtcccccttacacttttggccagggaaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 100 - DNA, 657 bp, Homo Sapiens
gaaattgtgctgactcagtctccaggcaccctgtcttcgtctccaggggaaagagccaccctctcctgcagggccagtcaaagtgttactagcagatacttagcctggtaccagcagaaacatggccaggctcccaggctcctcatctatggtacatccacgagggccactggcatcccagacaggttcagtggcggagggtctcagacagacttcactctcaccatcagcagactggagcctgaagattttgcagtgtattattgtcagcactatgatgactcaatttcgacgtacatttttggcccggggaccgagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 101 - DNA, 648 bp, Homo Sapiens
gacatccagttgacccagtctccatcttctgtgtctgcttctgtaggagacagagtgaccatcacttgtcgggcgagtcagggtattaacaacttattagcctggtatcagcagaaaccagggaaagcccctaagctcctgatctacgctgcatccaatttgcaaagtggggtcccatcgaggttcagcggcagtggatctgggacagatttcactctcactatcaacagcctgcagcctgaagattttgcaacctactattgtcaacaggctaacagtttccctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 102 - DNA, 651 bp, Homo Sapiens
gacatccagatgacccagtctccatcctccctgtctgcatctgtaggagacagagtcaccatcacttgccgggcaagtcagagcattagcagctatttaaattggtatcagcagaatccagggaaagcccctaagctcctgatctatggtgcatccaatttgcaaagtggggtcccatcaaggttcagtggcagtggatctgggacagatttcactctcaccatcagcagtctgcaacctgaagattttgcaacttactactgtcaacagagttacagtaccctcgcgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 103 - DNA, 651 bp, Homo Sapiens
gacatccagatgacccagtctccatcctccctgtctgcatctgtaggagacagagtcaccatcacttgccgggcaagtcagagcattagcacctatttaaattggtatcagcagaaaccagggaaagcccctaagctcctgatctatgctgcatccactttgcaaagtggggtcccatcaaggttcagtggcagtggatctgggacacatttcactctcaccatcagctctctgcaacgtgaagattttgcaacttactactgtcaacagacttacagaacccccacgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 104 - DNA, 645 bp, Homo Sapiens
gacatccagttgacccagtctccatcctccctgtctgcatctgtaggagacagagtcaccatcacttgccgggcaagtcagaacattaacaggtatttaaattggtatcagcacaaaccagggagagcccctgagctcctgatctatgctgcgtccactttacgaaggggggtcccatcaaggttcagtggcagtggatctgggacagatttcactctcaccatcagcagtctgcaacctgaagattttgcaacttactactgtcaacagagttacagtagagggacgttcggccaagggactaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 105 - DNA, 651 bp, Homo Sapiens
gacatccagatgacccagtctccatcctccctgtctgcatctgtaggagacagagtcaccatcacttgccgggcaagtcagagcattagcacctatttaaattggtatcagcagaaaccagggaaagcccctaagctcctgatctatgctgcatccactttgcaaagtggggtcccatcaaggttcagtggcagtggatctgggacacatttcactctcaccatcagcagtctgcaacgtgaagattttgcaacttactactgtcaacagagttacggaacccccacgtggacgttcggccaagggaccaaggtggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 106 - DNA, 657 bp, Homo Sapiens
cagactgtggtgactcaggagccctcactgactgtgtccccaggagggacagtcaccctcacctgtgcttccagcactggagcagtcaccactggttactatccaaactggttccagcagaaacctggacaagcacccagggcactgatttatagtacaagcaagaaacactcctggacccctgcccggttctcaggctccctccttgggggcaaagctgccctgacactgtcaggtgtgcagcctgaggacgaggctgagtattactgcctgctcttctatggtggtgctcagctgggggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttcataataa

SEQ ID NO: 107 - DNA, 651 bp, Homo Sapiens
gacatccagatgacccagtctccatcctccctgtctgcatctgtaggagacagagtcaccatcacttgccgggcaagtcagagcattggcaactatttaaattggtatcagcagaaaccaggaaaagcccctaagctcctgatctctgctgcatccagtttgcaaagtggggtcccgtcaaggttcagtggcagtggatctgggacagatttcactctcaccatcagcagtctgcatcgtgaagactatgcaacttactactgtcaacagagttacagtacccccccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaggtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 108 - DNA, 651 bp, Homo Sapiens
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacctccacgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 109 - DNA, 648 bp, Homo Sapiens
gacatccagttgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacctcgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 110 - DNA, 654 bp, Homo Sapiens
gaaattgtgttgacgcagtctccagccaccctgtctgtgtctccaggggaaagagccaccctctcctgcagggccagtcagagtgttaccagcaacttagcctggtaccagcagaaacctggccaggctcccaggctcctcatctatggtgcatccaccagggccactggtgtcccagccaggttcagtggcagtgggtctgggacagagttcagtctcaccatcagcagcctgcagtctgaagattttgcagtttattactgtcagcagtataataactggcctcccatattcactttcggccctgggaccaaactggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 111 - DNA, 651 bp, Homo Sapiens
gaaattgtgctgactcagtctccaggcaccctgtctttgtctccaggggaaagagccaccctctcctgcagggccagtcagagtgttagcagcagctacttagcctggtaccagcataaacctggccaggctcccaggctcctcatctacggttcatccaacagggccactggcatcccagacaggttcagtggcagtgggtctgggacagacttcactgtcaccatcagcagactggagcctgaagattttgcagtgtattactgtcagcagtatggtactgcaccctacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 112 - DNA, 654 bp, Homo Sapiens
cagtctgccctgactcagcctgcctccgtgtctgggtctcctggacagtcgatcaccatctcctgcactgcaaccagcagtgatattggggcttataactatgtctcctggtaccaacaccacccaggtaaagcccccaaagtcatcattactgatgttaataagcggccctcaggggtccctgatcgcttctctggctccaagtctggcaacacggcctccctgaccatctcagggctccagcctgaggatgaggctgagtattcctgctgctcatatgcaggcacctacagttatgtcttcggaactgggaccaaggtcaccgtcctgagtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataataa

SEQ ID NO: 113 - DNA, 654 bp, Homo Sapiens
ctttcttctgagctgactcaggaccctgctgtgtctgtggccttgggacagacagtcaggatcacatgccaaggagacagcctcagaagttattatgcaaactggtaccagcagaagccaggacaggcccctctatcagtcatctatggtaaaaacaaccggccctcagggatcccggaccgattctctggctccaactcaggaaacacagctttcttgaccatcactgggactcaggcggaagatgaggctgactattactgtaactcccgggacagcagtggtaattatcgggagctattcggcggagggaccaagctgaccgtccttggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctgcagaatgctcttaataa

SEQ ID NO: 114 - DNA, 651 bp, Homo Sapiens
gacatccagttgacccagtctccatccttcctgtctgcatctgtaggagacagagtcaccatcacttgccgggccagtcatggcattagcagttatttagcctggtatcaacagaaaccagggaaagcccctaacctcctgatctttcctgcatccactttgcaaagtggggtcccgtcaagattcagcggcagtggatctgggacagaattcactctcacaatcagcagcctgcggcctgaagattttgcaacttattactgtcaacaacttaatagttattccaggtgggcgttcggccaagggaccaaggtggaagtcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 115 - DNA, 651 bp, Homo Sapiens
gacatccagatgacccagtctccatcctccctgtctgcatctgtaggagacagagtcaccatcacttgtcgggcaagtcagagcattaggaggtatttaaattggtatcagaagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttaccgtacccaaggtctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 116 - DNA, 657 bp, Homo Sapiens
caggctgtggtgactcaggagccctcactgactgtgtccccaggagggacagtcactctcacctgtgcttccagcactggagcagtcaccactggttactatccaaactggttccagcagaaacctggacaagcacccagggcactggttcatagtacaagcaagaaacactcctggacccctgcccggttctcaggctccctccttgggggcaaagctgccctgacactgtcaggtgtgcagcctgaggacgaggctgagtattactgcctgctcttctatggtggtgctcaactgggggtgttcggcggagggaccaaactgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcggatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctgcagaatgctcttaataa

SEQ ID NO: 117 - DNA, 657 bp, Homo Sapiens
caggctgtggtgactcaggagccctcactgactgtgtccccaggagggacagtcactctcacctgtgcttccagcactggatcagtcaccagtggttactatccaaactggttccagcagaaacctggacaagcacccaggccactgatttctggtacaagcaacaaactctcctggacccctgcccggttctcaggctccctccttgggggcaaagctgccctcacagtgtcaggtgtgcagcctgaggacgaggctgtgtattactgcctgctctactatggtgttcctcagccagtggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcataataa

SEQ ID NO: 118 - DNA, 651 bp, Homo Sapiens
gacatccagttgacccagtctccatcctccctgtctgcatctgtaggagacagagtcaccatcacttgccgggcaagtcagagcattagcagctatttaaattggtatcagcagaaaccaggaaaagcccctaagctcctgatctctgctgcatccagtttgcaaagtggggtcccgtcaaggttcagtggcagtggatctgggacagatttcactctcaccatcagcagtctgcaacgtgaagactatgcaacttactactgtcaacagagttacagtacccccccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 119 - DNA, 648 bp, Homo Sapiens
gacatccagatgacccagtctccttccaccctgtctgcatctgtaggagacagagtcaccatcacttgccgggccagtcagagtattagcagttggttggcctggtatcaacagaaaccagggaaagcccctaagctcctggtctataagacgtctagtttagaaggtggggtcccatccaggttcagcggcagtggatctgggacagaattcagtctcacaatcttcagactgcagtctgatgattttgcaacttattactgccaacagtataatagttttccgtacacctttggccaggggaccaagctggagttcacacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 120 - DNA, 654 bp, Homo Sapiens
gaaattgtgttgacgcagtctccagtcaccctgtctttgtctccaggggaaagagccaccctctcctgtagggccagtcagagtgttagcagcggctacttagcctggtaccagcagaaacctggccaggctcccaggctcctcatctatggtacatccatcagggccactggcatcccagacaggttcagtggcagtgggtctgggacagacttcactctcaccatcagcagactggagcctgaagattttgcagtgtattactgtcagcagtatggtagctcacctctatactcttttggccaggggaccaaggtggacatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 121 - DNA, 651 bp, Homo Sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacggtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 122 - DNA, 648 bp, Homo Sapiens
gacatccagttgacccagtctccgtcctccctggctgcatctgtgggagacagagtcattattacttgccggtcaggtcagggcattaggaactatttaaattggtatcagcagaaacctgggaaagcccctaaactcctgatctatgctgcgtcctttttgcaaagtggggtcccatcaaggttcagtggcagtggatctgggacagatttcactctcaccatcagcagtctgcaacctgaggattttgcaacttacaactgtcaacagagttacagtgacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 123 - DNA, 645 bp, Homo Sapiens
gacatccagatgacccagtctccatcttccctgtctgcatctgtaggagacagagtcattatcacttgccgggcaagtcagagcgttaacaggtatttaaattggtatcagcagaaaccagggaaagcccctaaactcctcatctatgctgcatccagtttgcaaggtggggtcccatcaaggttcagtggcagtggatctgggacagatttcactctcaccatcagcagtctgcaacgtgaagattttgcaacttactactgccaacagagttacagaactcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 124 - DNA, 648 bp, Homo Sapiens
gacatccagatgacccagtctccatcctccctgtctgcatctgtaggagacggaatcaccatcacttgccgggcaagtcaaagcgttaggagctatttaaattggtatcagcagaaaccagggaaagcccctgagctcctgatctatgctgcatcccgtttgcaaagtggggtcccatcaaggttcagtggcagtggatctgggacagatttcactctcaccatcagcagtctgcagcctgaagattttgcaacttactactgtcaacatagttacagtacccctgtcacgttcggccaagggaccaaggtggaagtcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 125 - DNA, 651 bp, Homo Sapiens
ctttcttctgagctgactcaggaccctgctgtgtctgtgaccttgggacagacggtcagaatcacatgccaaggagacagcctcagacactcttatgcaagctggtaccagcagaagccagggcaggctcctatacttgtcatctatggtaaaaacatccggccctcagggatcccagaccgattctctggctccacctcggggaacacagcttccttgaccatcactggggctcaggcggaagatggcggtgactattactgtaactcccgggacaccagtactgaccattatgtcttcggagatgggaccagggtcaccgtcgtaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataataa

SEQ ID NO: 126 - DNA, 651 bp, Homo Sapiens
gacatccagatgacccagtctccatcctccctgtctgcatctgtaggagacagagtcaccatcacttgccgggcaagtcagagcattagcagctggttaaattggtatcagcagaaaccagggaaagcccctaacctcctgatctttgctgcatccactttgcaaagtggggtcccgtcaaggttcagtggcagtggatctgggacagatttcactctcaccatcagcagtctgcaacctgaagattttgcaacttactactgtcaacagagttacagtagttctgtgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagcgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 127 - DNA, 648 bp, Homo Sapiens
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggatagatttcactctcaccatcagcagtctgcaacctgaagattttgcaacttactactgtcaacagagttacactaccctctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 128 - DNA, 657 bp, Homo Sapiens
gaaattgtgctgactcagtctccaggcaccctgtctttgtctccaagggaaagagccaccctctcctgcagggccaatcagtatgttaacagcaaccacttagcctggtaccagcagaaacctggccaggctcccaggctcctcctttatggtgcatcaaggagggccactggcatcccagacagattcagtggcagtgggactgggacagacttcactctcatcatcagcagactggagcctgaagattttgccgtatatttctgtcagctgtatgatcactcacgtccgatgtacacttttggccaggggactaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 129 - DNA, 654 bp, Homo Sapiens
gaaattgtgctgactcagtctccaggcaccctgtctttgtctccaggggaaagagccaccctctcctgcagggccagtcagagttttagcagcggctacttagcctggtaccagcagaaacctggccaggctcccaggctcctcatctatggtgcatccaacagggccactggcatcccagacaggttcagtggcagtgggtctggaacagacttcactctcaccatcagcagactggagcctgaagattttgcagtgtattactgtcagcactatggtagctcacctcccatcaccttcggccaagggacacgactggagattaaacgaactgtggctgcaccatctgtcttcatcttcccgccatctgatgagcagttgaaatctggaactgcctctgttgtgtgcctgctgaataacttctatcccagggaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 130 - DNA, 651 bp, Homo Sapiens
gacatccagatgacccagtctccatcctccctgtctgcatctgtaggagacagagtcaccatcacttgccgggcaagtcagagcattagcagctatttaaattggtatcaacagaaaccagggaaagcccctaagctcctgatctttgctgcatccagtttgcaaagtggggtcccatcaaggttcagtggcagtggatctgggacagatttcagtctcaccatcagcagtctgcaacctgaagactttgcaacttactactgtcaacagagttacagttccctcgcgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 131 - DNA, 651 bp, Homo Sapiens
gacatccagttgacccagtctccatcctccctgtctgcatctgtaggagacagagtcaccatcacttgccgggcaagtcagagcattagcagctatttaaattggtatcagcagaatccagggaaagcccctaagctcctgatctatggtgcatccaatttgcaaagtggggtcccatcaaggttcagtggcagtggatctgggacagatttcactctcaccatcagcagtctgcaacctgaagattttgcaacttactactgtcaacagagttacagtaccctcgcgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 132 - DNA, 651 bp, Homo Sapiens
gacatccagatgacccagtctccatcctccctgtctgcatctgtaggagacagagtcaccatcacttgccgggcaagtcagagcattagcacctatttaaattggtatcagcagaaaccaggaaaagcccctaagctcctgatctctgctgcatccagtttgcaaagtggggtcccgtcaaggttcagtggcagtggatctgggacagatttcactctcaccatcagcagtctgcaacgtgaagactatgcagcttactactgtcaacagagttacagtacccccccgtacacttttggccaggggaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 133 - DNA, 657 bp, Homo Sapiens
caggctgtggtgactcaggagccctcactgactgtgtccccaggagggacagtcactctcacctgtgcttccagcactggagcagtcaccactggttactatccaaactggttccagcagaaacctggacaagcacccagggcactgatttatagtacaagcaagaaacactcctggacccctgcccggttctcaggctccctccttgggggcaaagctgccctgacactgtcaggtgtgcagcctgaggacgaggctgagtattactgcctgctcttctatggtggtgctcagctgggggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttcataataa

SEQ ID NO: 134 - DNA, 654 bp, Homo Sapiens
cagactgtggtgacccaggagccatcgttctcagtgtcccctggagggacggtcacactcacttgtggcttgagctctggctcagtctctgctcgttactaccccagctggtaccagcagaccccaggccagcctccacgcacgctcatccacagcacaaatactcggtcttctggggtccctgatcgcttctctggctccatccttgggaacaaagctgccctcaccatcacgggggcccaggcagatgatgaatctgattattactgtgtgctgtatatgggtagtggcccttgggtgttcggcggagggaccaagctgaccgtcctaggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttcataataa

SEQ ID NO: 135 - DNA, 663 bp, Homo Sapiens
gatgttgtgatgactcagtctccactctccctgcccgtcacccctggagagccggcctccatctcctgcaggtctagtcagagcctcctgcataggaatggatacaactatttgaattggtacctgcagaagccagggcagtctccacagctcctgatctatttgggttctaatcgggcctccggggtccctgacaggttcagtggcagtggatcaggcacagattttacactgaaaatcagcggagtggaggctgaggatgttgcgttttattactgcatgcaaggtctacgaactccgtacactttcggccaggggaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 136 - DNA, 648 bp, Homo Sapiens
gacatccagttgacccagtctccatcctccctgtctgcatctgtaggagacagagtcaccatcacttgccgggcaagtcagagtatcagcagctatttaaattggtataagcagagaccagggaaagcccctaagctcctgatctatgctgcatccactttgcagagtggggtcccatcaaggttcagtggcagtggatctgggacagatttcgctctcaccatcagcagtctgcaagctgaagattttgcaacttactactgtcaacagacttacagtaccctttggacgttcggccaagggaccaaggtggaaatcacacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 137 - DNA, 651 bp, Homo Sapiens
gaaattgtgttgacacagtctccaggtaccttgtctctgtctccaggggaaacagccaccctgtcctgcagggccagtcagagtgtcagtgatcgcgacttggcctggtatcaacaaaagtctggccagtctcccagacttctcatgtatggtggatccaccagggcccctggtataccggtcaggttcagtggcagtgggtctgggacagagttcactctcaccatcagcagcctgcagtctgaagattttgcaatttattactgtcaacactatcatgactggcctccgaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 138 - DNA, 648 bp, Homo Sapiens
gacatccagatgacccagtctccatcctccctgtctgcatctttagggggcacggtcaccctcacttgccggtcaagtcagttcattagtcgctatttaaattggtatcaacaacacccagggaaagtccctagactcctcatttctggcgcatcaagattgcaaaggggggtcccgtcaaggttcactggcggcgggtctgggacagacttcacactcaccataaagaatgtacagcctgacgatattgcaacatacttctgtcagcactcttacagaagtgggcgggcgttcggccaagggaccacggtggaggtgaaacgaactgtggctgcaccatctgtcttcatcttcccgccatctgatgagcagttgaaatctggaactgcctccgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 139 - DNA, 651 bp, Homo Sapiens
gaaattgtgttgacgcagtctccaggcagcctgtctttgtctccaggggaaagagccaccctctcctgcagggccagtcagagtgttagcagcagctacttagcctggtaccagcagaaacctggccaggctcccaggctcctcatctatggtccatccagcagggccactggcatcccagacaggttcagtggcagtgggtctgggacagacttcactctcaccatcagcagactggagcctgaagattttgcagtgtattactgtcagcattttggtaactcacggggaacgttcggccaagggaccaaggtggaaatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 140 - DNA, 654 bp, Homo Sapiens
gaaattgtgttgacgcagtctccaggcaccctgtctttgtctccaggggaaagagtcacactctcctgcaggcccagtcggtatattgccagcgactacttagcctggtaccaactaagacctggccaggctcccaaactcctcatctatggtgcctccagcagggccactggcatcccagacaggttcagtggcgttgggtctccgacagacttcactctcaccatcagcagactggagcctgaagattttgcgatgtattactgtcactattctggtggctcacctccgtacccttttggccaggggaccaggctggacatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 141 - DNA, 648 bp, Homo Sapiens
gacatccagttgacccagtctccatcctccctgtctgcatctgtaggagacagagtcaccatcacttgccgggcaagtcagtacattaacgtctacttaaattggtatcagcacaaagcagggagagcccctaagctcctgatctatgctgcatccaatttgcaaagtggggtcccaccaaggttcattggcagtggatctgggacagatttcactcttaccatcagcagtctgcaatctgaagatttcgcaacttactactgtctccagagtttcactgtccctcggactttcggccctgggaccaaagtggatgtcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 142 - DNA, 651 bp, Homo Sapiens
gaaattgtgttgacacagtctccaggcaccctgtctttgtctccaggggagagagccaccctctcctgcagggccagtcagagtgttagcagcgccttcttagcctggtaccagcagaaacctggccaggctcccagactcctcatctatggtgcctccagcagggccactggcatcccagacaggttcagtggcagtgggtctgggacagacttcactctcaccatcagcagactggagcctgaagattttgcagtgtattactgtcagcagtatggcagcttttcgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 143 - DNA, 657 bp, Homo Sapiens
cagactgtggtgacccaggagccctcactgactgtgtccccaggagggacagtcactctcacctgtgcttccagcactggagcagtcaccagtggttactatccaaactggttccagcagaaacctggacaagcacccagggcactgatttatagtacaagcaacaaacactcctggacccctgcccggttctcaggctccctccttgggggcaaagctgccctgacactgtcaggtgtgcagcctgaggacgaggctgagtattactgcctgctctactatggtggtgctcagcgttgggtgttcggcggagggaccatcctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttcataataa

SEQ ID NO: 144 - DNA, 657 bp, Homo Sapiens
cttaattttatgctgactcagccccactctgtgtcggagtctccggggaagacggtaaccatctcctgcacccgcagcagtggcagcattgccagcaactatatgcagtggtaccagcagcgcccgggcagttcccccaccactgtgatctatgaggataatcggagaccctctggggtccctgatcgcttctctggctccatcgacagctcctccaactctgcctccctcaccatctctggactgaagactgaggacgaggctgactactactgtcagtcttatgatagtaacaattgggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttcataataa

SEQ ID NO: 145 - DNA, 651 bp, Homo Sapiens
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccactttgcaaagtggggtcccatcaaggttcagtggcagtggatctgggacagatttcactctcaccatcagcagtctgcaacctgaagattttgcaacttactactgtcaacagtattacagaacgcccacgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 146 - DNA, 651 bp, Homo Sapiens
gaaattgtgttgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccccggtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 147 - protein, 127 aa, Homo Sapiens
EVQLVESGGGLVQPGGSLRLSCVASGFTFRSFDMNWVRQAPGKGLEWVSYINSRGSTIYYADSVKGRFTISRENAKNSLYLQMNSLRAEDTAVYYCARDLYGDYDPKSYYYYGMGVWGQGTTVTVSS

SEQ ID NO: 148 - protein, 125 aa, Homo Sapiens
EVQLVETGGGVVQPGRSLRLSCAASGFSFSNFGFHWIRQSPGKGLEWVAVIWYDGSNRFYADSVKGRFTISRDSSKNMLFLQMDSLRAEDTAVYYCAREISMKVVIRRHYVMDVWGHGTTVTVSS

SEQ ID NO: 149 - protein, 127 aa, Homo Sapiens
EVQLVETGGGLVQPGGSLRLSCAASGFSFRSYDMNWVRQAPGKGLEWVSYISGRGSTTYYADSVKGRFTISRDNAKNSLYLQMNSLRAEDTAVYYCARDLYGDYDPKSYYYYAMDVWGHGTTVTVSS

SEQ ID NO: 150 - protein, 125 aa, Homo Sapiens
EVQLVESGGGVVQPGRSLRLSCAASGFTFSNYGMHWVRQAPGKGLEWVAVIWYDGSNRFYADSVKGRFTISRDNSKNTLYLQMNNLRAEDTALYSCAREITTTVVVRRHYLMDIWGQGTTVTVSS

SEQ ID NO: 151 - protein, 126 aa, Homo Sapiens
EVQLVESGGGVVQSGRSLRLSCAASGFTFSNNGMHWVRQAPGKGLEWVAFIWYDGSNKYYADSVKGRFTISRDNSKNTLYLQMDGLRAEDSAVYYCAREEIAARLYSRYHYAMDVWGQGTMVTVSS

SEQ ID NO: 152 - protein, 125 aa, Homo Sapiens
QVQLVESGGGLVKPGGSLRLSCAASGFGFSAYSMNWVRQAPGKGLEWVSSITSTTTYYADSVKGRFSISRDNAKSTLYLRMNSLRAEDTAVYYCVREIAFRGSTYSRWSYYFDFWGQGTLVTVSS

SEQ ID NO: 153 - protein, 128 aa, Homo Sapiens
QVQLQESGPGLVKPSETLSLTCTVSGGSISSYYWSWVRQPPGKGLEWIGYIYYSGSTNYNPSLKSRVTIALDTSKNQFSLKLRSVTAADTAVYFCTRDWRQYGSAIRGSRYYYGMDVWGQGTTVTVSS

SEQ ID NO: 154 - protein, 129 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDMVTMVRGAYRNYYYYGMDVWGKGTTVTVSS

SEQ ID NO: 155 - protein, 127 aa, Homo Sapiens
EVQLVETGGGVVQPGRSLRLSCAASGFSFSNYGMHWVRQAPGKGLEWVAVIWFDGSIKYYVDSVKGRFTISRDNSKNTLYLQMNSLRAEETAIYFCARENSVLVPGTIRRRYYLDYWGQGTLVTVSS

SEQ ID NO: 156 - protein, 127 aa, Homo Sapiens
QVQLVESGGDLVQPGGSLRLSCAASGFTFRSYEMNWVRQAPGKGLEWVSYINSRGNTKYYADSVKGRFTISRDNAKKSLYLQMSSLRAEDTAVYYCARNLFGDYDLKSYYYNAMDVWGQGTLVTVSS

SEQ ID NO: 157 - protein, 127 aa, Homo Sapiens
QVQLVESGGGLVKPGGSLRLSCAASGFTFSSYAMNWVRQAPGKGLEWVSSISGTSSYIYYADSVKGRFTIFRDNAKSSVYLQMNSLRVEDTAVYYCARDRWWGMVRRVFPTYPFDYWGQGTLVTVSS

SEQ ID NO: 158 - protein, 125 aa, Homo Sapiens
QVQLVETGGGVVQPGRSLRLSCAASGFTFSSYGMHWVRQAPGKGLEWVAVIWYDGSNKDYADPVKGRFTISRDNSKNTLYLQMNSLRAEDTAVYYCAREIASRGYSRYLYYFDSWGQGTLVTVSS

SEQ ID NO: 159 - protein, 129 aa, Homo Sapiens
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARMRLTMVRGVITYYYYSMDVWGQGTTVTVSS

SEQ ID NO: 160 - protein, 127 aa, Homo Sapiens
QVQLQESGPGLVKPSQTLSLTCTVSGGSISSGSYYWSWIRQPAGKGPEWIGRIYTSGSTNYNPSLKSRLTISVDTSKNQFSLKLTSVTAADTAVYYCARAPSYYDSSGYRYWYIDLWGRGTLVTVSS

SEQ ID NO: 161 - protein, 126 aa, Homo Sapiens
QVQLVESGGGVVQPGRSLRLSCAASGFTFSSYGMHWVRQAPGKGLEWVAVIWYDGSNKDYADSVKGRFTISRDNSKNTLYLQMNSVRAEDTAVYYCARELSTQRGYSRYHYVMDVWGQGTTVTVSS

SEQ ID NO: 162 - protein, 121 aa, Homo Sapiens
QVQLVESGGGVVQPGRSLRLSCAASGFTFSNYGMHWVRQAPGKGLEWVAVIWFDGSNRDYADSVKGRFTISRDNSKKTLYLQMNSLRAEDTAVYYCATELARGRLRALEYWGQGTLVTVSS

SEQ ID NO: 163 - protein, 126 aa, Homo Sapiens
QVQLVQSGGGVVQPGKSLRLSCAASGFTFSTYGMHWVRQAPGKGLEWVAVIWYDGSNKYYADSVKGRFTISRDTSKNTLYLQMNSVRVEDTAVYYCARDLTTQRGYSRYHYVMDVWGQGTTVTVSS

SEQ ID NO: 164 - protein, 126 aa, Homo Sapiens
QVQLVESGGGVVQPGRSLRLSCAASGFTFSSYGMHWVRQAPGKGLEWVAVIWYDGSNKYYADSVKGRFTISRDNSKNTLHLQMNSLRAEDTAVYYCAREVGFGSGWSRYYYGMDVWGQGTTVTVSS

SEQ ID NO: 165 - protein, 130 aa, Homo Sapiens
EVQLVESGGGGVVQPGRSLRLSCAASGFTFSSYGMHWVRQAPGKGLEWVAVIWYDGSNKYYADSVKGRFTISRDNSKNTLYLQMNSLRAEDTAVYYCARESTLYSSSWYRRYYYYGMDVWGQGTTVTVSS

SEQ ID NO: 166 - protein, 129 aa, Homo Sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARESTLYSSSWYRRYYYYSMDVWGQGTTVTVSS

SEQ ID NO: 167 - protein, 128 aa, Homo Sapiens
QVQLVESGGGVVQPGRSLRLSCAASGFTFSSYGMHWVRQAPGKGLEWVAVIWFDGSNRYYGDSVKGRVTISRDNSKNTLYLRMNSLRAEDTAVYYCAREFYTRSGLWSQGYSYYMDVWGKGTTVTVSS

SEQ ID NO: 168 - protein, 129 aa, Homo Sapiens
EVQLVESGGGVVQPGRSLRLSCAASGFTFSNYGMHWVRQAPGKGLEWVAVIWYDGSNKYYADSVKGRFTISRDNSKNTLYLQMNSLRAEDTAVYYCAREMVSYSSSWYRRYYYYVMDVWGKGTTVTVSS

SEQ ID NO: 169 - protein, 123 aa, Homo Sapiens
EVQLVESGGGVVQPGRSLRLSCAASGFSFSNYGMHWVRQAPGKGLEWVAVISYDGSEKYYADSVKGRFTISRDNSKKALYLQMNSLRAEDTAMYYCKNKVGATRRAVVAFDIWGQGTTVTVSS

SEQ ID NO: 170 - protein, 127 aa, Homo Sapiens
EVQLVETGGGLVKPGGSLRLSCAASGFTFRSYSMNWVRQAPGKGLEWVSSIGSSSTYTYSADSVKGRFTISRDNAKNSLYLQMNSLRAEDTAVYYCARGEPLNYDYIWGGYRFTIHWGQGTLVTVSS

SEQ ID NO: 171 - protein, 129 aa, Homo Sapiens
QVQLVESGGGVVQPGRSLRLSCAASGFTFSSYGMHWVRQAPGKGLEWVAIIWFDGSNKYYADSVKGRFTISRDNSKNTLYLQINSLRAEDTAVYYCAKEHGYYSSSWYRNYYYYAMDVWGQGTTVTVSS

SEQ ID NO: 172 - protein, 121 aa, Homo Sapiens
EVQLVESGGGVVQPGRSLRLSCAASGFTFSSYGMHWVRQAPGKGLEWVAVIWYDGSNKDYVDSVKGRFTISRDNSKNTLYLQMNSLRAEDTAVYYCARELAKGRLRDLDHWGQGTLVTVSS

SEQ ID NO: 173 - protein, 129 aa, Homo Sapiens
EVQLVESGGGVVQPGRSLRLSCAASGFTFRTSAMHWVRQAPGKGLEWVAVIWYDGSNKYYADSVKGRFTISRDNSKNTLYLQMNSLRAEDTAVYYCAREMVSYSSSWYRRYYYYNMDVWGKGTTVTVSS

SEQ ID NO: 174 - protein, 125 aa, Homo Sapiens
QVQLVESGGGVVQPGTSLRLSCAASGFTFRTYGMHWVRQAPGKGLEWVAYIWYDGSNKYYADSVKGRFTISRDNSKSTLNLQMNSLRAEDTAVYYCAREIASRGYSRYLYYFDSWGQGTLVTVSS

SEQ ID NO: 175 - protein, 129 aa, Homo Sapiens
QLQLQESGPGLVKPSGTLSLTCAVSGGSMRSSNWWTWVRQPPGKGLEWIGEIHHGGSTNYNPSLQSRVTISVDKSKNRFSLKLSSVTAADTAVYHCARGRSYYDSSGHSFRGLVPFDIWGQGTMVTVSS

SEQ ID NO: 176 - protein, 128 aa, Homo Sapiens
QVQLQESGPGLVKPSETLSLICTVSGGSISSNYWSWIRQPPGKGLEWIGYIYYSGNTNYNPSLKSRVTISLDTSKNQFSLKLRSVTAADTAVYYCAREWRQYGSGIRGSRYYYGMDVWGQGTLVTVSS

SEQ ID NO: 177 - protein, 129 aa, Homo Sapiens
EVQLVESGGGVVQPGRSLRLSCAASGFTFSNHGMHWVRQAPGKGLEWVAVIWYDGSNKYYADSVKGRFTISRDNSKNTLYLQMNSLRAEDTAVYYCAREMASYSSSWYRRYYYYVMDVWGKGTTVTVSS

SEQ ID NO: 178 - protein, 125 aa, Homo Sapiens
QVQLVESGGGVVQPGRSLRLSCAASGFTFSTYGMHWVRQAPGKGLEWVAVIWYDGSQKYYVDSVKGRFTISRDNSKNTLYLQMNSLRAEDTAVYYCAREVAVRGVIRYYYGMDVWGQGTTVTVSS

SEQ ID NO: 179 - protein, 128 aa, Homo Sapiens
QVQLVESGGGVVQPGRSLRLSCAASGFSFSSYGMHWVRQAPGKGLEWVAIIWYDGSNKLYADSVKGRFTISRDNSRNTLYLQMSSVRAEDTAVYYCARDSVRGVSRWGTQKYYAMDVWGQGTTVTVSS

SEQ ID NO: 180 - protein, 126 aa, Homo Sapiens
EVQLVQSGGGVVQPGRSPRLSCAASGFTFSSYGMHWVRQAPGKGLEWVAFIWYDGSNKYYADSVKGRFTISRDNSKNTLYLQMNSLRAEDTAVYYCARDLRNHVFWSGYSTSFDYWGQGTLVTVSS

SEQ ID NO: 181 - protein, 129 aa, Homo Sapiens
EVQLVETGGGVVQPGRSLRLSCAASGFTFSSYGMHWVRQAPGKGLEWVAVIWYDGSNKYYADSVKGRFTISRDNSKNTLYLQMNSLRAEDTAVYYCAREMVSYSSSWYRRYYYYNMDVWGKGTTVTVSS

SEQ ID NO: 182 - protein, 129 aa, Homo Sapiens
QVQLVETGGGVVQPGRSLRLSCAASGFTFSSYGMHWVRQAPGKGLEWVAVIWSDGSNKYYADSVKGRFTISRDNSRNTLYLQMNSLRAEDTAVFYCAREQGGYSSSWYRRYYYYYMDVWGQGTTVTVSS

SEQ ID NO: 183 - protein, 125 aa, Homo Sapiens
EVQLVESGGGVVQPGRSLRLSCAASGFTFNTYGMHWVRQAPGKGLEWVAVIWYDGSNKYYADSVKGRFTISRDNSKNTLYLQMNSLRAEDTAVYYCAREVAVRGVIRYYYAMDVWGQGTTVTVSS

SEQ ID NO: 184 - protein, 123 aa, Homo Sapiens
QVQLVESGGGVVQPGRSLRLSCAASGFSFSNYGMHWVRQAPGKGLEWVAVISYDGSEKYYADSVKGRFTISRDNSKKTLYLQMNSLRAEDTAMYYCKNKVGATRRAVVAVDIWGQGTMVTVSS

SEQ ID NO: 185 - protein, 127 aa, Homo Sapiens
QVQLVESGGGLVQPGGSLRLSCVGSGFTFRSFDMNWVRQAPGKGLEWISYINSRGNTRYYVDSVKGRFTISRDNAKNSLYLQMNSLRAEDTAVYYCARDLYGDYDPKSYYYYGMDVWGQGTMVTVSS

SEQ ID NO: 186 - protein, 125 aa, Homo Sapiens
EVQLVESGGGVVQPGRSLRLSCEASGFTFSNYGMHWFRQAPGKGLEWVAIIWYDGSNKHYADSVKGRFTISRDNSKNTLYLQMNSLRAEDTAVYFCAREIASRGYSRYLYYFDSWGQGTLVTVSS

SEQ ID NO: 187 - protein, 125 aa, Homo Sapiens
QVQLVESGGGVVQPGRSLRLSCAASGFTFRTYGMHWVRQAPGKGLEWVAVIWYDGSNKDYADSVKGRFTISRDNSKNTLHLQMNSLRAEDTAVYYCAREIASRGYSRYLYYLDFWGQGTLVTVSS

SEQ ID NO: 188 - protein, 126 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSVRAEDTAVYYCARDLSTQRGYSRYYYVMDVWGQGTTVTVSS

SEQ ID NO: 189 - protein, 121 aa, Homo Sapiens
QVQLVESGGGVVQPGRSLRLSCAASGFTFSNFGMHWVRQAPGKGLEWVAVIWYDGSNKDYADSVKGRFTISRDNSKRTLYLQMNSLRAEDTAVYYCARELARGRLRDLDYWGQGTLVTVSS

SEQ ID NO: 190 - protein, 128 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREFYTRSGLWSQGYSYYMDVWGKGTTVTVSS

SEQ ID NO: 191 - protein, 129 aa, Homo Sapiens
QVQLVESGGGVVQPGRSLRLSCAASGFTFSSYGMHWVRQAPGKGLEWVAVIWFDGGNKYYADSAKGRFTISRDNSKNTLYLQMNSLRAEDTAVYYCARDASVLSGLVTRRLVYYGMDVWGQGTTVTVSS

SEQ ID NO: 192 - protein, 129 aa, Homo Sapiens
QVQLVESGGGVVQPGRSLRLSCAASGFTFSSYGMHWVRQAPGKGLEWVAFISYDGSNKYYADSVKGRFTISRDNSKNTLSLQMNSLRAEDTAVYYCAKEHGYYRSSWYRNYYYYGMDVWGQGTTVTVSS

SEQ ID NO: 193 - protein, 129 aa, Homo Sapiens
QVQLVESGGGVVQPERSLRLSCAASGFTFSSYGMHWVRQAPGKGLEWVAVISYDGSNKYYADSVKGRFTISRDNSKNTLYLQMNSLRAEDTAVYYCAKDEVGYSSSWYRRYYYYAMDVWGQGTTVTVSS

SEQ ID NO: 194 - protein, 128 aa, Homo Sapiens
QVQLVQSGGGVVQPGRSLRLSCAASGFTFSSYGMHWVRQAPGKGLEWVAVIWYDGSNKYYADSVKGRFTISRDNSKNTLYLQMNSLRAEDTAVYYCARETVVVAAKIRNHYYYAMDVWGQGTTVTVSS

SEQ ID NO: 195 - protein, 129 aa, Homo Sapiens
QVQLVESGGGVVQPGRSLRLSCAASGFTFSSYGMHWVRQAPGKGLEWVAGIWYDGSNKYYADSVKGRFTISRDNSRNTMYLQMNSLRAEDTAVYYCAREQGGYSSSWYRRYYYYNMDLWGQGTTVTVSS

SEQ ID NO: 196 - protein, 129 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNTLRDDDTAVYYCAREGTLYSSSWYRRYYYYGMDAWGQGTTVTVSS

SEQ ID NO: 197 - protein, 129 aa, Homo Sapiens
QVQLVETGGGVVQPGRSLRLSCAASGFTFSSYGMHWVRQAPGKGLEWVAGIWYDGSNKYYGDSVKGRFTISRDNSRNTLYLHMNSLRADDTAVYYCAREHGGSRSGWYTLRLAYYFDYWGQGTLVTVSS

SEQ ID NO: 198 - protein, 129 aa, Homo Sapiens
QVQLQESGPGLVKPSGTLSLTCAVSGGSIRGSNWWSWVRQPPGKGLEWIGEIHHGGSTNYNPSLKSRVTISVDKSKNRFSLKLSSVTAADTAVYYCARGTSYYDSSGYSFRGLVAFDIWGQGTMVTVSS

SEQ ID NO: 199 - protein, 129 aa, Homo Sapiens
QVQLVESGGGVVQPGRSLRLSCAASGFTFSTYGMHWVRQAPGKGLEWVAVIWYDGSNKNYADSVKGRFTISRDNSKNTLYLQMNSLRAEDTAVYYCARDLQGYRSSWYRMYYYYGMDVWGQGTTVTVSS

SEQ ID NO: 200 - protein, 127 aa, Homo Sapiens
EVQLVESGGGLVKPGGSLRLSCAASGFTFRSYSMNWVRQAPGKGLEWVSSIGSSSIYTYSADSVKGRFTISRDNAKNSLYLQMNSLRAEDTAVYYCARGEPLNYDYIWGRSRLTIHWGQGTLVTVSS

SEQ ID NO: 201 - protein, 129 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDWVTRSSNWYRNYYYYGMDVWGQGTTVTVSS

SEQ ID NO: 202 - protein, 124 aa, Homo Sapiens
EVQLVESGGGLVQPGRSLRLSCVASGFTFSSYWMHWVRQVPGKGLVWVSRINVDGKSTSYADSVKGRFTISRDNAKNTLYLQMNSLRAEDTAVYYCARDPRRFLEWARYGMDVWGRGTTVTVSS

SEQ ID NO: 203 - protein, 216 aa, Homo Sapiens
EIVLTQSPATLSLSPGERATLSCRASQSVSSYLAWYQQKRGQAPRLLIFNASNRATGIPARFSGSGSGTDFTLTISSLEPEDFAVYYCQQRSSWPPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 214 aa, Homo Sapiens
DIQMTQSPSPLSASVGDRVSITCRASRGISNSLAWYQQKPGKVPKLLIYAASTLQSGVPSRFSGGGSGTDFTLTISSLQPEDVATYYCHTYNSAPFAFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 216 aa, Homo Sapiens
EIVMTQSPGTLSLSPGERATLSCRASHTVSSGYLAWYQQKPGQAPRLLIYGASNRATGVPDRFGGSGSGTDFTLTISRLEPEDSAVYFCQQYGTSPGVTFGQGTRLEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 214 aa, Homo Sapiens
DIQLTQSPSSLSASVGDRVSITCRASQGISNSLAWYQQKPGKVPKLLIYAASTLQSGVPSRFSGGGSGTDFTLTISSLQPEDVATYYCQKYNSAPFAFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 215 aa, Homo Sapiens
DIQMTQSPSFLSASVGDRVTITCRASQGIRRYLAWFQQKPGKAPKLLIFSASTLQSGVPSRFSGSGSGTEFTLTISSLQPEDFATYYCQQLSSYPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 208 - protein, 214 aa, Homo Sapiens
DIQLTQSPSSVSASVGDSVTITCRASQAVSGWVAWYQQKPGKAPKLLIFGLSNLEDGVPSRFSGSGSATDFTLTITGLQPEDLATYYCLQANRFPLSFGGGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 216 aa, Homo Sapiens
EIVLTQSPGTLSLSPGERATLSCRASQNPRRNFLAWYQQKPGQAPRLLIYAASTRATGIPDRFSGSGSGTDFTLTIDRLEPEDSAVYYCQVYGSSPLYTFGQGTKVEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 216 aa, Homo Sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPTLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 214 aa, Homo Sapiens
DIQLTQSPSSLSASVGDRVTITCRASQSISSYLNWYQQKPGEAPKLLIYVASTLQSGAPSRFSGSGSGTDYTLTISSLQPEDSATFYCQQTYSPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 217 aa, Homo Sapiens
EIVLTQSPGTLSSSPGERATLSCRASQSVTSRYLAWYQQKHGQAPRLLIYGTSTRATGIPDRFSGGGSQTDFTLTISRLEPEDFAVYYCQHYDDSISTYIFGPGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - protein, 214 aa, Homo Sapiens
DIQLTQSPSSVSASVGDRVTITCRASQGINNLLAWYQQKPGKAPKLLIYAASNLQSGVPSRFSGSGSGTDFTLTINSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 215 aa, Homo Sapiens
DIQMTQSPSSLSASVGDRVTITCRASQSISSYLNWYQQNPGKAPKLLIYGASNLQSGVPSRFSGSGSGTDFTLTISSLQPEDFATYYCQQSYSTLALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 215 aa, Homo Sapiens
DIQMTQSPSSLSASVGDRVTITCRASQSISTYLNWYQQKPGKAPKLLIYAASTLQSGVPSRFSGSGSGTHFTLTISSLQREDFATYYCQQTYRTPTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 213 aa, Homo Sapiens
DIQLTQSPSSLSASVGDRVTITCRASQNINRYLNWYQHKPGRAPELLIYAASTLRRGVPSRFSGSGSGTDFTLTISSLQPEDFATYYCQQSYSRGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 215 aa, Homo Sapiens
DIQMTQSPSSLSASVGDRVTITCRASQSISTYLNWYQQKPGKAPKLLIYAASTLQSGVPSRFSGSGSGTHFTLTISSLQREDFATYYCQQSYGTPTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 217 aa, Homo Sapiens
QTVVTQEPSLTVSPGGTVTLTCASSTGAVTTGYYPNWFQQKPGQAPRALIYSTSKKHSWTPARFSGSLLGGKAALTLSGVQPEDEAEYYCLLFYGGAQLGVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 219 - protein, 215 aa, Homo Sapiens
DIQMTQSPSSLSASVGDRVTITCRASQSIGNYLNWYQQKPGKAPKLLISAASSLQSGVPSRFSGSGSGTDFTLTISSLHREDYATYYCQQSYSTPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 215 aa, Homo Sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTSTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 214 aa, Homo Sapiens
DIQLTQSPSSLSASVGDRVTITCRASQSISSYLNWYQQKPGKAPKLLIYAASSLQSGVPSRFSGSGSGTDFTLTISSLQPEDFATYYCQQSYSTSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 216 aa, Homo Sapiens
EIVLTQSPATLSVSPGERATLSCRASQSVTSNLAWYQQKPGQAPRLLIYGASTRATGVPARFSGSGSGTEFSLTISSLQSEDFAVYYCQQYNNWPPIFTFGPGTKLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 215 aa, Homo Sapiens
EIVLTQSPGTLSLSPGERATLSCRASQSVSSSYLAWYQHKPGQAPRLLIYGSSNRATGIPDRFSGSGSGTDFTVTISRLEPEDFAVYYCQQYGTAPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 216 aa, Homo Sapiens
QSALTQPASVSGSPGQSITISCTATSSDIGAYNYVSWYQHHPGKAPKVIITDVNKRPSGVPDRFSGSKSGNTASLTISGLQPEDEAEYSCCSYAGTYSYVFGTGTKVTVLSQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 225 - protein, 216 aa, Homo Sapiens
LSSELTQDPAVSVALGQTVRITCQGDSLRSYYANWYQQKPGQAPLSVIYGKNNRPSGIPDRFSGSNSGNTAFLTITGTQAEDEADYYCNSRDSSGNYRELFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 226 - protein, 215 aa, Homo Sapiens
DIQLTQSPSFLSASVGDRVTITCRASHGISSYLAWYQQKPGKAPNLLIFPASTLQSGVPSRFSGSGSGTEFTLTISSLRPEDFATYYCQQLNSYSRWAFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 215 aa, Homo Sapiens
DIQMTQSPSSLSASVGDRVTITCRASQSIRRYLNWYQKKPGKAPKLLIYAASSLQSGVPSRFSGSGSGTDFTLTISSLQPEDFATYYCQQSYRTQGLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 217 aa, Homo Sapiens
QAVVTQEPSLTVSPGGTVTLTCASSTGAVTTGYYPNWFQQKPGQAPRALVHSTSKKHSWTPARFSGSLLGGKAALTLSGVQPEDEAEYYCLLFYGGAQLGVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPAECS

SEQ ID NO: 229 - protein, 217 aa, Homo Sapiens
QAVVTQEPSLTVSPGGTVTLTCASSTGSVTSGYYPNWFQQKPGQAPRPLISGTSNKLSWTPARFSGSLLGGKAALTVSGVQPEDEAVYYCLLYYGVPQPVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 230 - protein, 215 aa, Homo Sapiens
DIQLTQSPSSLSASVGDRVTITCRASQSISSYLNWYQQKPGKAPKLLISAASSLQSGVPSRFSGSGSGTDFTLTISSLQREDYATYYCQQSYSTPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - protein, 214 aa, Homo Sapiens
DIQMTQSPSTLSASVGDRVTITCRASQSISSWLAWYQQKPGKAPKLLVYKTSSLEGGVPSRFSGSGSGTEFSLTIFRLQSDDFATYYCQQYNSFPYTFGQGTKLEFTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 216 aa, Homo Sapiens
EIVLTQSPVTLSLSPGERATLSCRASQSVSSGYLAWYQQKPGQAPRLLIYGTSIRATGIPDRFSGSGSGTDFTLTISRLEPEDFAVYYCQQYGSSPLYSFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 233 - protein, 215 aa, Homo Sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - protein, 214 aa, Homo Sapiens
DIQLTQSPSSLAASVGDRVIITCRSGQGIRNYLNWYQQKPGKAPKLLIYAASFLQSGVPSRFSGSGSGTDFTLTISSLQPEDFATYNCQQSYSDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 213 aa, Homo Sapiens
DIQMTQSPSSLSASVGDRVIITCRASQSVNRYLNWYQQKPGKAPKLLIYAASSLQGGVPSRFSGSGSGTDFTLTISSLQREDFATYYCQQSYRTRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 214 aa, Homo Sapiens
DIQMTQSPSSLSASVGDGITITCRASQSVRSYLNWYQQKPGKAPELLIYAASRLQSGVPSRFSGSGSGTDFTLTISSLQPEDFATYYCQHSYSTPVTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 215 aa, Homo Sapiens
LSSELTQDPAVSVTLGQTVRITCQGDSLRHSYASWYQQKPGQAPILVIYGKNIRPSGIPDRFSGSTSGNTASLTITGAQAEDGGDYYCNSRDTSTDHYVFGDGTRVTVVGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 238 - protein, 215 aa, Homo Sapiens
DIQMTQSPSSLSASVGDRVTITCRASQSISSWLNWYQQKPGKAPNLLIFAASTLQSGVPSRFSGSGSGTDFTLTISSLQPEDFATYYCQQSYSSSVYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 214 aa, Homo Sapiens
DIQMTQSPSSLSASVGDRVTITCRASQSISSYLNWYQQKPGKAPKLLIYAASSLQSGVPSRFSGSGSGIDFTLTISSLQPEDFATYYCQQSYTTLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 217 aa, Homo Sapiens
EIVLTQSPGTLSLSPRERATLSCRANQYVNSNHLAWYQQKPGQAPRLLLYGASRRATGIPDRFSGSGTGTDFTLIISRLEPEDFAVYFCQLYDHSRPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 216 aa, Homo Sapiens
EIVLTQSPGTLSLSPGERATLSCRASQSFSSGYLAWYQQKPGQAPRLLIYGASNRATGIPDRFSGSGSGTDFTLTISRLEPEDFAVYYCQHYGSSPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 215 aa, Homo Sapiens
DIQMTQSPSSLSASVGDRVTITCRASQSISSYLNWYQQKPGKAPKLLIFAASSLQSGVPSRFSGSGSGTDFSLTISSLQPEDFATYYCQQSYSSLALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 215 aa, Homo Sapiens
DIQLTQSPSSLSASVGDRVTITCRASQSISSYLNWYQQNPGKAPKLLIYGASNLQSGVPSRFSGSGSGTDFTLTISSLQPEDFATYYCQQSYSTLALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 215 aa, Homo Sapiens
DIQMTQSPSSLSASVGDRVTITCRASQSISTYLNWYQQKPGKAPKLLISAASSLQSGVPSRFSGSGSGTDFTLTISSLQREDYAAYYCQQSYSTPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 217 aa, Homo Sapiens
QAVVTQEPSLTVSPGGTVTLTCASSTGAVTTGYYPNWFQQKPGQAPRALIYSTSKKHSWTPARFSGSLLGGKAALTLSGVQPEDEAEYYCLLFYGGAQLGVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 246 - protein, 216 aa, Homo Sapiens
QTVVTQEPSFSVSPGGTVTLTCGLSSGSVSARYYPSWYQQTPGQPPRTLIHSTNTRSSGVPDRFSGSILGNKAALTITGAQADDESDYYCVLYMGSGPWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 247 - protein, 219 aa, Homo Sapiens
DVVMTQSPLSLPVTPGEPASISCRSSQSLLHRNGYNYLNWYLQKPGQSPQLLIYLGSNRASGVPDRFSGSGSGTDFTLKISGVEAEDVAFYYCMQGLRTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 214 aa, Homo Sapiens
DIQLTQSPSSLSASVGDRVTITCRASQSISSYLNWYKQRPGKAPKLLIYAASTLQSGVPSRFSGSGSGTDFALTISSLQAEDFATYYCQQTYSTLWTFGQGTKVEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - protein, 215 aa, Homo Sapiens
EIVLTQSPGTLSLSPGETATLSCRASQSVSDRDLAWYQQKSGQSPRLLMYGGSTRAPGIPVRFSGSGSGTEFTLTISSLQSEDFAIYYCQHYHDWPPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 214 aa, Homo Sapiens
DIQMTQSPSSLSASLGGTVTLTCRSSQFISRYLNWYQQHPGKVPRLLISGASRLQRGVPSRFTGGGSGTDFTLTIKNVQPDDIATYFCQHSYRSGRAFGQGTTVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - protein, 215 aa, Homo Sapiens
EIVLTQSPGSLSLSPGERATLSCRASQSVSSSYLAWYQQKPGQAPRLLIYGPSSRATGIPDRFSGSGSGTDFTLTISRLEPEDFAVYYCQHFGNSRGTFGQGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 216 aa, Homo Sapiens
EIVLTQSPGTLSLSPGERVTLSCRPSRYIASDYLAWYQLRPGQAPKLLIYGASSRATGIPDRFSGVGSPTDFTLTISRLEPEDFAMYYCHYSGGSPPYPFGQGTRLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 253 - protein, 214 aa, Homo Sapiens
DIQLTQSPSSLSASVGDRVTITCRASQYINVYLNWYQHKAGRAPKLLIYAASNLQSGVPPRFIGSGSGTDFTLTISSLQSEDFATYYCLQSFTVPRTFGPGTKVDVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - protein, 215 aa, Homo Sapiens
EIVLTQSPGTLSLSPGERATLSCRASQSVSSAFLAWYQQKPGQAPRLLIYGASSRATGIPDRFSGSGSGTDFTLTISRLEPEDFAVYYCQQYGSFSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - protein, 217 aa, Homo Sapiens
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQRWVFGGGTILTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 256 - protein, 217 aa, Homo Sapiens
LNFMLTQPHSVSESPGKTVTISCTRSSGSIASNYMQWYQQRPGSSPTTVIYEDNRRPSGVPDRFSGSIDSSSNSASLTISGLKTEDEADYYCQSYDSNNWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 257 - protein, 215 aa, Homo Sapiens
DIQMTQSPSSLSASVGDRVTITCRASQSISSYLNWYQQKPGKAPKLLIYAASTLQSGVPSRFSGSGSGTDFTLTISSLQPEDFATYYCQQYYRTPTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 215 aa, Homo Sapiens
EIVLTQSPATLSVSPGERATLSCRASQSVSSNLAWYQQKPGQAPRLLIYGASTRATGIPARFSGSGSGTEFTLTISSLQSEDFAVYYCQQYNNWPRYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - DNA, 23 bp, Artificial
  Synthetic primer sequence
cgttctttttcgcaacgggtttg

SEQ ID NO: 260 - DNA, 23 bp, Artificial
  Synthetic primer sequence
aagaccgatgggcccttggtgga